SEQUENCE LISTING (130 sequences)

SEQ ID NO: 1 - DNA, 1787 bp, Homo sapiens
gtgaatctctggggccaggaagaccctgctgcccggaagagcctcatgttccgtgggggctgggcggacatacatatacgggctccaggctgaacggctcgggccacttacacaccactgcctgataaccatgctggctgccacagtcctgaccctggccctgctgggcaatgcccatgcctgctccaaaggcacctcgcacgaggcaggcatcgtgtgccgcatcaccaagcctgccctcctggtgttgaaccacgagactgccaaggtgatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgtggtggaagttgggttaggagtacggagatggagattggctcccaactcctccctatcctaaaggcccactggcattaaagtgctgtatcc

SEQ ID NO: 2 - DNA, 1790 bp, Homo sapiens
gtgaatctctggggccaggaagaccctgctgcccggaagagcctcatgttccgtgggggctgggcggacatacatatacgggctccaggctgaacggctcgggccacttacacaccactgcctgataaccatgctggctgccacagtcctgaccctggccctgctgggcaatgcccatgcctgctccaaaggcacctcgcacgaggcaggcatcgtgtgccgcatcaccaagcctgccctcctggtgttgaaccacgagactgccaaggtgatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgtggtggaagttgggttaggagtacggagatggagattggctcccaactcctccctatcctaaaggcccactggcattaaagtgctgtatccaag

SEQ ID NO: 3 - DNA, 1457 bp, Homo sapiens
cctggccctgctgggcaatgcccatgcctgctccaaaggcacctcgcacgaggcaggcatcgtgtgccgcatcaccaagcctgccctcctggtgttgaaccacgagactgccaaggtgatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgtggtggaagttgggttaggagtacggagatggagattggctcccaactcctccctatcctaaaggcccactggcattaaagtgctgtatccaag

SEQ ID NO: 4 - DNA, 24001 bp, Homo sapiens
agcttatgtctctgagccttgggaaacagtgagggtcagcccggctccccacgtgcttctgggcagctttggtattggagcaggtgcaaactcgggactagggcaggaccccctgagaggcgactgagcaaggccatcccgactcatgtttccttggccctgcccggggcacagcatcctgcccacatccctgcagccctggctccttcctaggggctctgaggaggcagcacttggtcatctggtcacagttgctgcagggcagttcttggccccagctgtaggtaaagtactgtatgttgtaattttttgaaagataacacgttcacacaactcagaattgaaatgccacagacattccccctgctccgcccctttcccccggatacccagtttctcccggaggcagccaatgatctcagaggctgtatacccccccagagttattttatgcatatcaaggaaagtctacatagaggactgtttctggggtacccagatgcagcgtcaaatgccatggaatactacagtgaggacattatcctttcaagctttcaaatcagagcaagggaaaggtcgatgctagagtttctctagcacccatgaagccctctccctttttctactgagttttactttacaggcaacagcaggcttcaagcttggggtcattgtcgggcaacagtatctggcaagaattcaatgtctttttctcatagtcattgtattttggcctctttctatttatggcaactgagagagaaagcttattcctagatatatgtatttaagtaaaaaataaatgaattcatggaaacatattaagcaattatccagataacataagggatggcaaaaatggtgcagatggtggaggggagacaagtagaagttggggtgctcttgttgaatgtctggctctgaactctagaggaggccgcaggggctgggcaggaaggaggtgaatctctggggccaggaagaccctgctgcccggaagagcctcatgttccgtgggggctgggcggacatacatatacgggctccaggctgaacggctcgggccacttacacaccactgcctgataaccatgctggctgccacagtcctgaccctggccctgctgggcaatgcccatgcctgctccaaaggcacctcgcacgaggcaggcatcgtgtgccgcatcaccaagcctgccctcctggtgtgtaagtatcagtgcatctgtctgccctgccaggggtcttttcatggacacccactatgccaggagcctccctggcctgaagccagccctgaagccggctgccacactagcccagagagaggagtgccctgggagggagatgggctgagtggagctgtcatcaccccctcctgacctcgccttcaaggtcaagttctttggtgagaaggtcctagctgcattgcaaacagccaggtatagggatttgtgtttgtctgagacccagaatcactggggttcgagttagggttcagatctgagccaggttagggggttgagtcagggggtaaagattaggaggttggtgtatatttggtgttgggggtcactctatggccaaagtcaggggttgccatgagctcaggtgacggaggctccatcactgactgttgtgtgactttggccagctcccctgccctctctgggcctcagtctcttgctcaatataataagggtatagggaggctaaatgatacaatttctaaaatagagtatcgccaagttcaaaagccagaattatagacccccaggactacagacagtgtcacagcatcgtctgggtgaggctagggttagtgtgcggctgggctcagggctgccccatttgctaggatcgtggggttcccatgtgtcaggatccagaggctagggtatgatcaggatctctagctggggtcagggtcagagctctctgtgtcccctagaattgccatcaaccttaaacccagagggaggcccagtccaacccctcagctttaagacctgctgggagcctcatctcagagaggctgagtcatggccaaggccagttgggggtgggagcagggggcttggtgtgggcctgcagcccctcatccactgccctcccctctagtgaaccacgagactgccaaggtgatccagaccgccttccagcgagccagctacccagatatcacgggcgagaaggccatgatgctccttggccaagtcaagtatgggttgcacaagtgagtcgggcctcgggtgtgaccaggctgggggtagggaggcgggaggaacagcctggggcttgcccccagcccacagggaggaaaggcagcagctgggggactcaggtctctccccttgatttggaaccagagcctgacaccttccctacccccaccctccatcccctggtgccctggggggatttattggagtgtatcaacctctccaacagcccctctaagagtcaggcttcaaagggtcctttcccactgccctgggaagatggaggtcttatttcggggtgaatggggggtagggtagaaaaatctcaacaaaataagtattttttaaaaaaatgtataaatgttgttttcttatagtacagacaggtcttgctatgttggccaggttggtcttgaactcttggcttagccagtcccccaccccaagcctaaaattagtatcttgactttatttgggatgatggtaacagtcaagggtcggttgtgggccaggtgcttttacaaacaacatcctcacctttatcatatatttttttctttcttgtttttcattttttgagacagagtctccctctgtcacttaggctggagtgcagtggcacaatctcagctcgttgcaacctccgtctctgggttcaaggaattttcgtgcctcagcctccagagtagctggaattacaggcacacgtcaccacatccgcgtaatttttgtatttttagtagagatggggtttcatcatgttggccagactggtctccaactcctgacctcaagtgatccgcccacctcagtctcccgaagtgctggggttacaggcgtgagccaccacgcctggccctttatctatcttaatctttattttatttatttatttatttatttatttatttatttatttatttatttatttattgagatggagtctctcactgtcacccaggctggagtgcagtggcacgatctcagctcactgcaacctccacctcccgggttcaagacgttctcctgcctcagcctccctagtagctgggattacaggtgcccaccaccacgcccggctaatttttgtattcttagtagagacggggtttcaccatgttgacctggttggtcttaaactcctgacctcaagtgatccacccgcctcggcctcccaaagtcctgggattgcaggcgtgagccacagcgtccggcctatcttaatctttatcatatattttagcctcttctgtaaaagaggataatgccatcgcccggatgaccctgcttaacaaacacggaaacaggagccgatagaggtttagcagcctggcggagctcatgtgcaaactcagctcagtatcggaaactcagccaccttcatggcacctcaggctgccctggagcctgggtcccggtctgtgtgtcggtctgaggccccaagggtggggcagacattgttcagggaccttgtctttgtcttaaagccaatctcctcctcctccccggccctgctaaacacgctccccaggggtggtctctgctacccgcgcacattgggccttgagtcttggtaaatgtttctgagccaccagggggcgccagcggtgctggatgccacttcgccttttgcaggaaaactcccgccgctagcgggtgctgtggggcgtcccaggtctcgcgcagccacgccctctgtgtgaccttgggcttcagtttaggcttctgtaaaagagaataatgggcaaggtccagtggctcatgcctgtgatcccagcactttgggaggccgaggtaggaggattacctcagccagggttcaagaccacctgtctctataaaaaatttaaaaattagctgggcatggtggtgcgtgcctgcagtcccagctactcaggaggccaggggaggtcaaggctgcggtgagctatgattgcatcactgcattccagcctgggtgacagagcaagactctgttgctaaaaaataaaagaggataatgctatcctagacctatgtgttgcaagaggtcagacatggcaaaagcgttgagaagttaaagaaatagtatccttaataaatcagcaataattcagtaagaccagactgccatagactagatgtcatagtcattaatatataactgttatcccaaacgtatatgagtggggtagaaataaaatatgattggccataactatgattggtcatgattaatgttagatgaagttggataatgggtaagatgagaattcttcacactattctctctaaagcgaatggaataaaatagaacagaacagaccgggaaagccagatatatgtggattctccttgctgggcacagtgtgtcatagtggcttataatatgggatttggagtgggatggacccaggatcaaattcccttctactgaggttttctgaagccacataagctgtctgagccatttctgtctcagttcagtgggtacagcaatagtgactacctcgtgggtagctgtgaggattaaaccaggaaatgtgtataaactctaagcacagtgcctgccacacagcatgtgtttggtacgtgatggccgcttctattgtgtcatattccatgtgctcaagccgggccctgggggagagttcagcaagggtagagggatatgagagtgaatgtggcccactggcctccctgcctgcagtctctcctctctgatccttgcttcctaagggtcttcactgagccccacctctgaccaagtccccggcggcacaggcagcacagatcctctgctgtacctacaccaagaccccagggccaacgcttctcccattgtgcagctccacctctgcccccggggcctagctgaggcctcctccaagctgagccacctactatcttccaaactcccaccctccatctggtgtcacctcctcccagaagccctctgtatcagtttcctgttttctgccataaaaaaaatcactccaagtttagtggcttaacatgacacaagtttaccattttacagtttaaaaggctagaagtccaccatggccctccctgagctaagatcaagctgtcagcagaaccagattccttctgggggctataggggagaattaattccctcgctttctgcagcttctagaggcttcccccattccttggcttccctccatctacttttatttagggcgagggaagcttcctgctctgactttctcatttatggtattctcccctgctttccagcagctgtgattgtcccaagtcctgtcctctggttctctaggccagaaaaactatgatctaccagagctcaagaacttgaaagggccttttcccactgtcccttctcacagtgagagccggagcccagcttcgtgctagaatctgtgaaaatggagaaatgcaccctgtgctgttctcttcctccacccggcatctcccctgcagcccggctgggtttttttgtttgtttgtttgtgggtttttttgtttttttgtttgtttttgagacagagttttgcacttgtcgcccaggctggagtgcaatggcacgatctcgactcactgcaacttctgcctcctgggttcaagcgattctcctgcctcaacctcccgagcagctggaaccacaagcatatgccaccactcccggctaatcttttttttttttttttatgcatttttagtagagatggggtttcaccatgctggccaggctggtctcgaactcctgacctcagatgatccacctgcctcggccttccaaagtgctgggattataggcgtgagccacggcctggcccagtctgactgttttcacttactctctaatgtctcaggtagtgtttattgtattctgtgcggaggtcaagaatttagtcatccaaggaggttagtgtattaggagcttacgcagctattaccagaagagactccactcaaaaatttggtacgtggctgggcgcagtggctcaaacctataatcccagcactttgggaggccgaggcgggcagatcacctgaggtcaggagttcaagaccagcctggccaacatggtgaaaccctatctctattaaaagtacaaaattagctgggcatgatggtgcatgcccataatctcagctacttgggaggccaaggcaggagaatcactgaacccaggaggtggagactgcagtgagctgagattgcccgctgcactccagcctgggcagcaagagcaaaattctgtcacacaaacaaacaaacaaacaaaaaaaacgggttgggcatggtggctcatgcctgtaaccccaacactttgggaggctgaggtgggggaatcacctgaggtcaggagttcaagaccagcctggcaacatggtgaaaccccctctctactaaaagtacaaaaattagctggatgtcgtggcgggagcctgtaatttcagctactagggaggctgaggcaggagaatcacttgaacccaggaggcagaggttgcagtgagccgagatcgtgccactgcactccagcctgagcaacagagccagactcagtctcaaaaaaaaaaaaaaaaacaatggtatgagctttaaatttctgtagaactgaaagaggattggagattgtagtagggtctaattctaaattagagcatttagaataatgtggagtctggtcaacatagtgagacccccccaatctgcacaaaaaagacaaaaattagccaggcagtgttggtgcatgcctgtggtcccagctattccagaggctgaggcaggaagatcgttggaattcgaggctgcagtgaactgtgattacacaactgcactctggcctgggcaacagagagccaccctatctcaaacaagaaagaacaaactaaaaaaaaaaaaagaagaagaagaatttggagcaatctagttgtttcctataccagaagtctttgccaatctatttttttttaattttatttttaatttttgtgagtacatagtaggagtatatatttatggggtacatgagagtacatagtaggagtatatatttatggggtacatgagatgttttgatatagacctgcaacgttttgtcaatctattttttttttggcgacagtcttgctctgttgcccaggctggagtgtaatggcgcgatctcagctcactgcaacttccgcctcgtgggttcaggctattctcctgccttagcctccaaactagctgggactacagacgcccaccatcatgcccggttaatttttttgtatttttgtggagacgagatttcaccatgttggccaggctggtcatgaactcctgacctcaggtgatctgccctcctcggcctcccaaagtgctaggattacaggtgtgagccaccacgctggcccaccaacctatttttgatctaactttacctctggtggtgtattgatactcttccgaatcaattttcacccttcttcatataccatttacatttctaagaatttgtccatttcatttcatctagttaacctcatttgttgacatacaattgttcgtagtattctctcataattccttttatttctgtaagactggtaataatggccttgcttttatttctgtttttagtaatttgagtcttcttctttttttttttttttgccagtctagctaaaggtttgttgattttgttgatcttttggaaaaaccagctattgttttcaccgattgttgtttttgttctattatttcattcataggcactaagatcctgattacttccttccttctgctagctttgggcttagtttgcccttatttttcaagttccataaggtggaaagtttgattattgagttgagatctttctcttttttaaagataggcatttctagatataaatctccctgtgagcacggttccctccatcttcagcacaccagggttgactctctccgggcgttcttccctggtcacctctccccttcctctcctcttctgcctcctcttccacttttcggtaccctgtgattgtattgggaccacccagataacctaggatcatctccccacctaccccaaggtccttaacttaaccatactttcatatgggtaacacgagttgagtgtggtacccagggtttgacatgttgggtaacatatttgcaggttctgtggattaggaggacattttgggggccatgattctatcttccaccctcgcctagacaaaattggaggctcactccttgggctccctggatgacccccaacatccttcctcacttccattccttcccagcatccagatcagccacttgtccatcgccagcagccaggtggagctggtggaagccaagtccattgatgtctccattcagaacgtgtctgtggtcttcaaggggaccctgaagtatggctacaccactgcctggtggtaagcattcctgtcagctgatgccccatgccctggccctctctgggctggagggctgaatgagggtcctgggtccttggctctttccaggctgggtattgatcagtccattgacttcgagatcgactctgccattgacctccagatcaacacacagctgagtatgtgtcaagcgtcctctggggaagtgggagctggactccagggcttggcctcagcagagggggaggttgtgcaggcagagggttctggggccaccaaaggaggcccagcctgggaagtttgcaggggtggggaccccagagctggccaagctcttgactggcctgggcagcatgtggataccatctgatagcggaggctgccctgaggtcatgtcgggtctccctgcagcctgtgactctggtagagtgcggaccgatgcccctgactgctacctgtctttccataagctgctcctgcatctccaaggggagcgagagtaagtacaccaccctgtggcccccattcctgctcgtgcccatcctgttagtgtgtccacggctccttccaggctcaaccccacacagggcatgcttgtgggtggccaaacctgagggcagcaataccttcagtggggtcacttcctaccccctcccatcaatacaccctcaaaggctggaaacaacaataaccaacagctagtaactaacagctattaagaacttgctgtgtgcaaagcactattccaagcccttttcatgaattaattgattttgtccttaaaaccaaccctaggatatagattctgttatcatcccctttttacatatgggtaaactgagtcacagagaggttagaaaggaaaagctcatatctacggagtgcatcctgcattccaagcaccacactaactcagagataaaactctagccaagctaagtaacttgctgaggacacacaactcgccactaagggatgggagtaggatttgaacccagcattctctgaccccagaagctgagttcctagatactttactctcctgcttcccagggtggggctttttgtcttggccaacaccctctgtcaaggagctgtgggtaaccccattgcacagaggaagataacaaggtttggagagtccctagtcatgttaccaatgccaaacctggaaggcagaagggaactggtgggtggggtctggagaggagccctctattcaggccattttctgactctggagcagacggatacatgtatgaatttggactctagacacgttctcgtgtgtgtgacaggtgtgagcgtcacaggagctgggccctcccgaggaattctgggatggtgccacagttaattcttgggtctgaggctccgtgttctcagctgcaaaatgggagtgataattcttacttcctgagctacaagagtcagggccaacagagccatgaacggtgcctggtacacactaggcgctccatggatgcacaggactggtcaggggctcattgtggtgcttgctgccttcaggcctgggtggatcaagcagctgttcacaaatttcatctccttcaccctgaagctggtcctgaagggacaggtgagtgaggctggctgactccctgtggtccagggccatgcccaggaggctggatccctttcctccctgcctttccctgagaaggtgccactcccaccttctccatgtggccagtcccctgtgccggtccccagcactgccaccaccacgcagctggaaggaggcactgcctctggcctcctttcctgcctggaaagcacctgctctgtctgccccagatctgcaaagagatcaacgtcatctctaacatcatggccgattttgtccagacaagggctggtgagtgcgtttctgtctgcatgcctcagaagacagcagtgggagccagaaagccacctgctgcactatgtggccttgggactgtcactcttcctgtctaggtcccatgggctctatctggctctgacacttgatgattagttatgagcatactttggcaaatctctgcccctttgggctgcagcctcacaagctgtgtggcgttgggcaagtctatagaactcaggacaaatgggtgattaagtccaagaggactccaagattctcctggaagtagattaggaaaaaagataattagattgctcacatggctgggcactcatccatgtactgtactctcctatgcagtacagagcagagctgggtttcagcccaagtcttggactctgctctgaaccaaccttctagaagggctctacctacccagacagacagacttgggaaaagagagaatgaaaaagtgccacacccctccccgcacacccaggtcccactttacagaggggaacactgaggctggagggttgggtagctgtgtggatgcaggggagcggtgactcagggcaattcccccatcctgaggccctgcgttgatcttttcctcctgcagccagcatcctttcagatggagacattggggtggacatttccctgacaggtgatcccgtcatcacagcctcctacctggagtcccatcacaaggtaggagttgtgggagggtggggcagggcccagcttccccaggggagttggtccttttttgtgctctgacaaccccgtcccccagcttcaaccttatggcagccaagagtcctggggcgctcctcctcattcctgatgctcctccgcattcctgatgctgcgaggagggcaggccacagcgacgtgcccctgacccctctctgcaggcaccagggctgcccactacaaggatcccagcaaagcaccagctccttcctagagggcttattcggcttctgtcatcctctacagcagtggattgtggcccccccccagggggtactgacaaaagctttggaccctctattacttaggatatagatttctgtaacaaagaaatccgaaataatagctgcttaaacacaataaacagttatttccctctgttgcgttctgagcatctgcagccccgcgggatcaggaggctgcagggtgtcaggcacgcaggccccttccacgtgttctcagccagccccaggcatgacctcatcccagggtcctggctgactggcctgaccaccacacccactctgctcacagcccgttggcctgaacctgatcgcaggaccccaccctagctgcaagggaaactgaggaatttatttcagctaaatattgagaccagctaaaagttaagtggtacagaatggagggagagattggagaagactttagggcggacccagccatctttgtaacagagcctctccctggaaaactggacagaaggatcatttcagaggttcatggccactcctgaagcctgttcacaacccatgggctggatgatctagtggggcgggggactgggcagcagcttgttttcctgattttggcctccaggagccgatggtcaatggactgccctctgcaggggcagggctggtggtcagctggggcggggtgggagctggaggtccgtggtcaccagctgccctgactaatgtcgttacttgaatataaccctgtgaaggcaggaaccacgtctgtctggttcacttcccacggtggttgagacatagtgggcactccggaagtatttgttgaatgagtgaaagccccgctgggggaaactgggtacagctctttcctcagtttccccatctgcactctgggctgaatgctggggctcctcccaatctccctgaagctggacctgagcccagtagggacacacagggtccagccagcgtcctggcttcctccagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggtgagtgggtggggctgggctgctaggggatccagatggcatgtggtatgtgtgtgtgtgcacacgcatggggaggagggaggaaactcggaaacttggtggtgggcaaaagaactaagctggagcaatagcagtgaagtccagactgggcacagtggctcacacctgtaatcccaatcctttgggaggctgagatgtagcaggacgaaccgcagacaaaactcctcagacactgagttaaagaaggaaagagtttattcagccgggagcatgggtaagactcctgtctcaagagcggagctctccgagtgagcaattcctgtcccttttaagggctcacaactctaagggggtctgcatgagagggtcgtgatctattgagcaagtagcaggtacgtgactgggggctgcatgcaccggtaatcagaacgaaacagaacaggacagggatttttacaatgctctttcatgcaatgtctggaatctatagataacataactggttaggtcaggggtccatctttaactaccaggcttaggtcaggtaggcccaggcctggtttcgggtctggttccttggtttcgggtctggtctctaggcgttgggctacctgcctttagtttcgcttctctttctttttctgagtataaaacaatataaaacaatatgagagggtctgtctctcttctctcagagacaggagggtccctggaggccaggagttcaagaccagcctgggcaacatggggagacccctgtctctacaaaaataaaaataagtttaaaaatcagctgtgcacggcggtgcatacctatagtcccaactactcaggtggctgaggagggaggatcacttgagcccaagaggttgaggctgcagtgagctatgatggcaccactgcactccagtgtgggtgacagagtaggactccatctcaagaaaaaaatacagtccagcatttattggagtcaactatgtgttgccagatagatagatggatagatagacagagtgatcaatagatttaggtatagatataacttggcacgtagtaactgtgcattaaatataagttgatattagacttacatttattgcagttatatttgctacactttgttttgttgcactcatcctgtgaaataagtactgttacctctgttttgcaaacaaaaaaaccacaaagctcagagaaggtaggtgacttactgaagatcacacagcctgtaagaggtggccccaaagcctgtactcttcacctatactgtactagaaatgcttaggtagtttccaggctgtgcttgtcattgaactcatgaggaaactgaggcccacagaggggaagagacttgtgcgaggtcacacagcatgtgtggggcacagtggggagcagaagccaggcctccagccgggacaggggttccctgttccacaccctgcccagagcatctcacatgttgtctgggaggttgggagttgcgtctgaggaggggtccagtccttgaaactgcccttggtccctgcgaagttttcttctgaggagtggactttactccacccaccctccaacttcctcatttcttttcaggcagtgctggagacctggggcttcaacaccaaccaggaaatcttccaagaggtaactgccccctgcccctgtgtggggtttatctcacgtaccccaatcctgctctggcttcaagagcccccacacagccaataacaccaccaatggcaacaattataacagcgaacacagctcctactcggttgttcggcatggagtgaagcacttagtgtgtgtgacttccttcagtgctcacaccgaccctatgagtggggcggtcaaactgtccccattttacacacagggaaacttagtgaatggcaaggctgggtttgagcccagctctattgcccccaaagataaggctccattccctgctccatttcccaggcatagggacttgtagggggctggaaccccaggatcaactctgggctcagagggccccagcaataagtgactgttgattactcctgatcccaaagctgacttcaggcaagctccttggaggtcgcagccccttcttgctatgcccagtggcaatgatgttcataatcccactcctcagtgcagggttccactaagaacccatgatctcctacctcaaatggacctcatgctttctgagtaagcctccctcagctttctggtcacctcactccccccacccactgcaatgacttcttcaggccttccctgccatcctcaaatctccagctgccccctcctgtctaccttccacttccctctccacacacaacctgcttaccagagagctgagcagagccaccaacagaacttcccccccacgtcgctgctcccagtcgcaacccactcacccacacctgcgccttcctgcggccgttctcctgttctaacagaggacggtccctcttcccttctgaactcaggccgtccagccttccccggctctgtgatgcctctctccttgcacatacacaactcacaatattgggcaggcccctgaccggctcttctcccttagccccttgtctgagtcctgttcctttgatgtcctatttctagtctttccatttctctctgtctctacctcctccccaggctaccattatctctagtggggacccatctgtagctcccggtgggtctccctgtgtcctgataccctccaataatctgttccccacagcagccagaacaatcttttcaagaaataaaactggtcattcacctgcctgcttaaagccggtgtaggatacaggctaaacgctctcccatggcctccagggccctgcattttctcatcctgtctacttcttcatctagttgtttcccacccagcgtaattgtaactgtttagttgaaagttgacgaggtcttttgtcagctctagaaatttcttttcttttctttcttttttttttttttaagacagagtcttgctctgtggcccaggctggagtgcagtagcacaatctcaactcactgcaacctccaccttccgggttcaagcaattctcctgcctcagtgctgaaattacaggtgtgagccacagcacccagcccatcagctctggaaatttcttcatcactctctcttccatgattgccccatccctattttctccatgcctacctcctggaatttccatgagacacacgttgggccatctcattccattttccgtgtctcttagttgctctgtcgtgttttccatctcttttatctctatcctaaattctggatagtttcttcaagtatctttttagttcattaattctttttacagctgtgtcttatttgtttacttacccactgagattttcattttattttatttaattaattaatttatttatttttgagacagagtctcgctcttgttccccaggctggagtgcagtggcgcaatctcagctcactgcaacctacgcctcctgtgttcaagcaattctcgtgcctcagcctcccgattggctggggttacaggggtgcaccactgtgcccagctcatttttttgtatttttagtagagacggggtttcaccatgttggccaggctggtcttgacctcctgacctcaaatgatcctcctgcctcagcctcccaaagtgccgggattacaggtgtgagctcccgcaccccgccggccctttctttcttttgagctgagctatgtatttaatgaaaccttttcttgtgtatccagcggttcagtggtttcttggggcatgtgatctccttgagcccagtcccacaggctgtaatcgaaggtcctcaggccacactccccagccccgctggctgcctgcgcattccttatcttggccatgttccttccacagggctcttgtccacactgtcccctctgccagaatggtttgccctcccccatgccatctgctcctcctcctgccacctcagggaaggcctctcccacctccctgactaggtcaggtcccctcgtgccacagcctggtgtgtctctcctttaaatactcctcagctgcagttttacgttctgttctgggatgccctggaaaatgctgggctcctgcagatatggcccgcagctcctgcgctctctgctctatccccacccctagcccagctgctacagcagcttccctcggagggttaccatgcagtaggtcctgttctaagctctttccacagattatctcattccatcctcaggacaaccctatgaggtaggatctatgattatccccattttacagatgaggaaagtgaggcccagaaggctaagtgaaggcatcctcagcacatcctgagagaggagttcagggtaggaatagcttcactagcacacagatgagaaactgaggcccagagaggacaagagtcctaatttgcacagcctggggcctgggcactaccccgagctacttccttttccccagccctggggcccggagccagctttgtccttcccatctccgagggcatggactgctgctgccctgatgggcccctgtcctggccatgggacccctgtcttccacaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggtgaggcgggtgcagggagaggtggtggtgggggaacctgactcacatatgggccgcagagggcaggggcctgggggtctctgaagcctccagatccttctcaccacctctgctggcactggttgtctcttgcacatggctccttacaatcaaaatcacatcatgcaagtaacgagggggtacacacgtggtttccacagcttaggtaatattttctctcttttcttatttttattttttttagagacagggtctcactctgtcatccaggctggagtgcagcctcacactcatagctcactgcagcctcgaatcctgggctcgagtgatcctcccacctcagccctcctgagtagctgggaccacagacaaacaccaccacacctggctaatttaaaaaaaaaaatttccttagaggcggggtcttgttatgttgtccaggctgttctcaaactcctggcctcaagcaattctcctgccttggcctcctaagttgctggggttataggcatgagccaccatgccaagcatatgttctttcattcttatttttatttattatttatttatttacttatttatttttttgacacagagtctcactgtgttgcccaggctggagtgcggtggcacaatctcggctcactacaacctctgcctcccaggttccagcgattctcatgcctcagcctcccaggtagctgggattacaggcatgcgctaccacgcctgactaattttttgtatttttagtagagacagggtttcgctatgtgggcccagctggtctgagactcctggtttcaagtgatctgcccgcctcagcctcccaaagtgctgggattacaggcatgagccaccacgcccagccaatattctcctccttaagctgagcagtggacaatggtgtttattacccgagtccacaaccctttattcattagggccacaagggtttccaaattcagaacttctgtgaatttgtaaaagtagtatagtgtatagacactggatatattgcagaacaccccaatagggtgtggggtggcaccccacaatcaattctcctcaaatgcctcaattggcatttctcctcatatgaagcaagataaataccatacatagcttcctagacaatggccaggttttgccaacaaaacttaagaaaaaacttgtagttttcagatcctgtttgattttataattacaaataaggaattaagaatcagtattatctttaatgctccttagaatatcttcaatttttttttttgagacagagtctcactctgtcacccaggctggagtgcagtggtgcaatctcggctcactgcaacctccaccttcctggttcaagcgattcgcgtgcctcagcctccccagtagctagctggcactacaggcgcgtgccaccacgcccagctaatttttagtagaggcggggtttcaccaggttggccaggccagtttcaaagtcctgacctccagtgatccacctgcctcagcctccaaaagtgctgggattacaggcatgagccactgtgctcggccaatatttcttaatacattaagaactaaagacaagagcgatacaatcacattgtggaaaatatagaaaaatagaaagaagaaaaccactgtcaccataacccccttctccaggggccttcgatgtggtgactgggtgcatttccttctggtgctttgttatttttaaataaattttttatcttggagtaactttatatttgaataaaagttgcaaaatacagagtgttcccatatatcccccacccacttcccgctttgtccacatcttacataaccatgctacagtgtcatagcggagagattaacattggtgcatgactgttaaccaaactctaaactttatttgaatctccccagtttttccactgatgataccctttttcttttccaagatctgatccaggatagcacattgtatttagccgttcttttttgttgttgtttgtttgtttgtttgtctgtttgtttctgcccaggctggagtgcagcggtgatcctcccacctcagcttctcgactagagatgtgcactaccacgtatttttttgtagagacagggttttgccacattgcccaagctggtctcaaatccctgggctcaagtgatcctcccaccttggcctcccaaagtgttgggattataggcatgagccactgtgcccagtttatttagccttttaaatatctctttaatttttgtattactttccttttcatttttctgttttattccttttggctttctcatgtggatgtagacatattattttatttacttatttttttgttgttaagaatctgaaacattgagcatttgttaaaaaaaagaattattcggccgggcacggtggctcacgcctgtaatcccagcactttgagaggctgaggtgggtggatcacatggtcaggagttcaagaccagcctggccaatatgatgaaaccccatctctactaaaaatacaaaaattagccggacatggtggcacatgcctgtagtcctagctacttgggaggctgaggtaggacaatcgcttgaacctgggacgtggaggttgcagtgagctgagatcgtgccactgccctccagcctgggcaacagagtgagactgtctcaaaaacaaaaaaagaaaagaaaagaaaaagaaagtgacttctcaggtcctaaccccaaagccacaggtgctggggaactttcctcggttttcagaagagcagtagctaagcctggttcccgtgtcatccttgcctctccagtccctcagtggaaagaatcaggggccctgagctaggagggttgctctctgcttcgggaagagccctggctcacagcaaatttggtttctctccccaggatatcgtgactaccgtccaggcctcctattctaagaaaaagctcttcttaagcctcttggatttccagtatgtgctgcagagaagagagggggcggtcaactccgcaaacctctccctggccccttggagtcaggcacagggcggggtgttggtggggaaatgtggcccctttcttctggggcatatgggctgactgcagggagataagaccctgcctagatagaatcttcgtggggaagaaggggctccaggaagaatggagggctgccaggaagaagggcctggcaggaggagagcgctgcccgagcaaaggcctggccgccagaatagcaaatctcaagggaatagcaaatctcaagagagtgccccaaagggcctgagctatgagacagaagcactggctgctattcttagagtttctttcccaggggatgttacaggagggggcccaatggagggtcaaattatcatcgcttttttatttcaggattacaccaaagactgtttccaacttgactgaggtaggtagtcttggatagactgggggaaataagtcctgtgggacctcctgccttaaagaaagcaggcggagggccctaaaggaaatcaggcaaccagaccaaaagaatgtggaccaggtggtccatgctgtgtctcttgtgacccttcttctccctgccatgtcttttgggagagcccttgtgttgcaaaaatgagagtgtggtggtatggattggggtttaggcagaacagtactggccaagcagcgcctccctggacctcaattttccctctgtggaatgggctagcaatcctgggcctccccagggcgaaggaaagaccactcaggaagggcaccgtctggggcaggaaaacggagtgggttggatgtatttttttcacggatgggcatgaggatgaatgcttgtccaggccgtgcagcatctgccttgtgggtcacttctgtgctccagggaggactcaccatgggcatttgattggcagagcagctccgagtccgtccagagcttcctgcagtcaatgatcaccgctgtgggcatccctgaggtcatgtctcgtaagtgtgggctggaggggaaactgggtgccgaggctgacagagcttcccatttcaccttgtgggcccttcccaggcagagcttcaggtgcccctcttcccagtcattgatacttagcggtcctggccccctttcctctccctgctggtggtattgcacgccaatgactcggccagatgcccagacccctgttcttggtttacctgcagaatattatctttgccaccccgcgggatggctcaacccactttcaggatgcaggtctcctaatagcaacctgatatagcagaaagacccctgggctgggagtctgagacctagttctagcccagccctgaacctcagtttccctttctgtgaaacaagaatgttgaacttgatgattcccaattttccttttgaccttgaaatggtagaatatttatccctttgaggtgactcggatggtagactctcagacaccatagcacacgtgtgctgggggtattttggaccaggctctgctgagagctttctgctcccttccccacagagtatgttgagctgactgtgctttccttttgaacatctgcttgtccacatggcttaggtaggagaggaagggcgtggaaactggaatgatcctagtggggtgtcttggcatctcttggcctcattttccccatctgaaccatgaagctaaaactaggggatgtggattaaatggttcctacaactacttgcaaggagaccactctgtgtggttgcaaagaacactttgagaagctgtgtgggaaagtttccttcctagcagggtagactcagctaactgcaggtcatgtggccattgtggatgggttgggagctcaagtttggggcagaagggaattttttttggcagcagagtggcaagccctgccgccaggcaaactctgctcttcctcatcctcagaagcacttgctcactctgctaaatcaaagtgaaacgcatgtttacagaatattggtccaaaagggtctcagcatctcccactacccagggtggcagagcctcgggccggccttgctccccaagaagggctgactggggctctgtcccctgccccagggctcgaggtagtgtttacagccctcatgaacagcaaaggcgtgagcctcttcgacatcatcaaccctgagattatcactcgagatgtgagtacaaagcccccctcaccagcccctgttcctggggagagaggcccagacaggattcctggggtgactgggggctgttggggagacagacagaggggcctctaccagcttggctccctcctggtggcctgggagtcagcccagctcgcccctctctcctactgcccctcccttcagggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgtggtggaagttgggttaggagtacggagatggagattggctcccaactcctccctatcctaaaggcccactggcattaaagtgctgtatccaagagctgcggagtccttcttctgtggctggcgggtagagggggggggaagggattgtctcaccagtgccgtccacctcttttcagcccttccaaggcagctgcccccaaaccctccaagcttcatgatgactggaggaagaaatccaaacttctctccttgggactcacgatcctccctgatcaggtccctggataccttccaaatttatcccctttaacccagcactctccttgtctgaccagcttcctgcaggagctgtcacacactctctgtcccttggggttccagtgctggacagaaagtgagggaggaccagggcttcagatcctgaggatggtgggaggggaaggggccctcagaggtctggtttctggtagaaatggaggaagacagagcctttaaaaaggcaaacgactttattccagaagataaactgctcacgagcctgagcttatttccagctcacctttttttttttttttttttttgagacggagtctctctgtgacccaggctggcatgcagtggcacaatcccagctcactgcaacctccacttcccaggttcaagcaattctcctgcctcagcctctcaagtatttaggattacaggcacccgccaccacacctagctagtttttgtatttttagtagagacggggtttcaccatgttggccaggctggtctcgaactcctgacctgcagtgatccacctgcctcaaccccccaaagtgctcatgttaaatatggtgaaccccaagtttttcttcaaagaatcagtatgtctgtatgttcagctctcttattctttgattctccattttaaagtttaacttcctggttctcttcgcccccttgcttctaatttcagtaaacaaccttttccatcagttttattcagtagttcacacctgttcccctggtcacttgctccatcctgactcatcccagtcacctgctttgacctgagtcacccctggtcacctgctctgatgtaagtcacctttagttacccgttcctaactgtccttcctgccaaactact

SEQ ID NO: 5 - DNA, 1870 bp, Oryctolagus cuniculus
gcctgtcccaaaggcgcctcctacgaggctggcatcgtgtgtcgcatcaccaagcccgccctcttggtgttgaaccaagagacggccaaggtggtccagacggccttccagcgcgccggctatccggacgtcagcggcgagagggccgtgatgctcctcggccgggtcaagtacgggctgcacaacctccagatcagccacctgtccatcgccagcagccaggtggagctggtggacgccaagaccatcgacgtcgccatccagaacgtgtccgtggtcttcaaggggaccctgaactacagctacacgagtgcctgggggttgggcatcaatcagtctgtcgacttcgagatcgactctgccattgacctccagatcaacacagagctgacctgcgacgctggcagtgtgcgcaccaatgcccccgactgctacctggctttccataaactgctcctgcacctccagggggagcgcgagccggggtggctcaagcagctcttcacaaacttcatctccttcaccctgaagctgattctgaagcgacaggtctgcaatgagatcaacaccatctccaacatcatggctgactttgtccagacgagggccgccagcatcctctcagatggagacatcggggtggacatttccgtgacgggggcccctgtcatcacagccacctacctggagtcccatcacaagggtcacttcacgcacaagaacgtctccgaggccttccccctccgcgccttcccgcccggtcttctgggggactcccgcatgctctacttctggttctccgatcaagtgctcaactccctggccagggccgccttccaggagggccgtctcgtgctcagcctgacaggggatgagttcaagaaagtgctggagacccagggtttcgacaccaaccaggaaatcttccaggagctttccagaggccttcccaccggccaggcccaggtagccgtccactgccttaaggtgcccaagatctcctgccagaaccggggtgtcgtggtgtcttcttccgtcgccgtgacgttccgcttcccccgcccagatggccgagaagctgtggcctacaggtttgaggaggatatcatcaccaccgtccaggcctcctactcccagaaaaagctcttcctacacctcttggatttccagtgcgtgccggccagcggaagggcaggcagctcagcaaatctctccgtggccctcaggactgaggctaaggctgtttccaacctgactgagagccgctccgagtccctgcagagctctctccgctccctgatcgccacggtgggcatcccggaggtcatgtctcggctcgaggtggcgttcacagccctcatgaacagcaaaggcctggacctcttcgaaatcatcaaccccgagattatcactctcgatggctgcctgctgctgcagatggacttcggttttcccaagcacctgctggtggatttcctgcagagcctgagctagcagggagctggagacacaagacacgctgacgtctccgcccatcggggtggaggtcagggagtgggtcggaggacgggcgatggctcccaactccttctgtcctgaagacccctagcatgaaagcagcataccctgggcaggcatctggctgagcggctaagccactggtcaggacacctgcgtctagggtgctccggatcccagctgcctgctaacgtgcaccctggggagcagccgtgctggttcccgccagccacatcggagacccagactgaggtcctggctcctggctttagcctgcccagtgagtggcagctaaatctctctggctgtctctctctgcctctcaagtaaacgaatatctcaaaaacaagag

SEQ ID NO: 6 - DNA, 1508 bp, Macaca fascicularis
atgctggctgccaccgtcctgaccctggccctgctgggcaatgtccacgcctgctccaaaggtacctcacacaaggcaggcattgtgtgccgcatcaccaagcctgccctcctggtgttgaaccaagagactgccaaggtgatccagtctgccttccagcgagccaactacccaaatatcacaggcgagaaggccatgatgctccttggccaagtcaagtatgggttgcacaacatccaaatcagccacttgtccatcgccagcagccgggtggagctggtggaagccaagtccattgatgtctccattcagaacgtgtctgtggtcttcaaggggaccctgaagtatggctacaccactgcctgggggctgggcattgatcagtccgttgacttcgagatcgactctgccattgacctccagatcaacacacaactgacctgtgactctggtagagtgaggactgatgcccctgactgctacctgtctttccataagctgctcctgcatctccaaggggagcgagagcccgggtggatcaagcagctgttcacaaacttcatctccttcaccctgaagctggtcctgaagggacagatctgcaaagagatcaacatcatctccaacatcatggccgattttgtccagacaagggctgccagtatcctttcagatggagacatcggggtggacatttccctgacaggtgatcccatcattacagcctcctacctggagtcccatcacaagggttatttcatctataagaatgtctcggaggacctcccactccccaccttctcgcccgcactgctgggggactcccgcatgctgtacttctggttctccgagcaagtcttccactccctggccaaggtagctttccaagatggccgcctcatgctcagcctgatgggagacgagttcaaggcagtgctggagacctggggcttcaacaccaaccaagaaatcttccaggaggttgtcggcggcttccccagccaggcccaagtcaccgtccactgcctcaagatgcccaggatctcctgccaaaacaagggagtcgtggtcaattcttcggtgatggtgaaattcctctttccacgcccagaccagcaacactctgtagcttacacatttgaagaggatatcatgaccaccgtccaggcctcctattctaagaaaaagctcttcttaagcctcttggatttccagattacaccaaagactgtttccaacttgactgagagcagctccgagtccgtccagagcttcctgcagtcaatgatcaccactgtgggcatccctgaggtcatgtctcggcttgaggcagtgtttacagccctcatgaacagcaaaggcctgagcctcttcgacatcatcaatcctgagattatcactcgagatggcttcctgctgctgcagatggactttggcttccctgagcacctgctggtggatttcctccagagcttgagctagaagtctccaaggacgtcaggatgggg

SEQ ID NO: 7 - DNA, 21 bp, Artificial Sequence
  Forward primer
caacggatttggtcgtattgg

SEQ ID NO: 8 - DNA, 26 bp, Artificial Sequence
  Reverse primer
ggcaacaatatccactttaccagagt

SEQ ID NO: 9 - DNA, 21 bp, Artificial Sequence
  Probe
cgcctggtcaccagggctgct

SEQ ID NO: 10 - DNA, 19 bp, Artificial Sequence
  Forward primer
gaaggtgaaggtcggagtc

SEQ ID NO: 11 - DNA, 20 bp, Artificial Sequence
  Reverse primer
gaagatggtgatgggatttc

SEQ ID NO: 12 - DNA, 20 bp, Artificial Sequence
  Probe
caagcttcccgttctcagcc

SEQ ID NO: 13 - DNA, 20 bp, Artificial Sequence
  Probe
tggaatcatattggaacatg

SEQ ID NO: 14 - DNA, 20 bp, Artificial Sequence
  Forward primer
ggcaaattcaacggcacagt

SEQ ID NO: 15 - DNA, 20 bp, Artificial Sequence
  Reverse primer
gggtctcgctcctggaagat

SEQ ID NO: 16 - DNA, 27 bp, Artificial Sequence
  Probe
aaggccgagaatgggaagcttgtcatc

SEQ ID NO: 17 - DNA, 23 bp, Artificial Sequence
  Forward primer
tgttctagagacagccgcatctt

SEQ ID NO: 18 - DNA, 21 bp, Artificial Sequence
  Reverse primer
caccgaccttcaccatcttgt

SEQ ID NO: 19 - DNA, 24 bp, Artificial Sequence
  Probe
ttgtgcagtgccagcctcgtctca

SEQ ID NO: 20 - DNA, 20 bp, Artificial Sequence
  Forward primer
tgctggacccaacacaaatg

SEQ ID NO: 21 - DNA, 20 bp, Artificial Sequence
  Reverse primer
tgccatccaaccactcagtc

SEQ ID NO: 22 - DNA, 26 bp, Artificial Sequence
  Probe
ttcccagtttttcatctgcactgcca

SEQ ID NO: 23 - DNA, 16 bp, Artificial Sequence
  Forward primer
gacggcgagcccttgg

SEQ ID NO: 24 - DNA, 21 bp, Artificial Sequence
  Reverse primer
tgctgtctttgggaccttgtc

SEQ ID NO: 25 - DNA, 24 bp, Artificial Sequence
  Probe
ccgcgtctcctttgagctgtttgc

SEQ ID NO: 26 - DNA, 17 bp, Artificial Sequence
  Forward primer
gccatggagcgctttgg

SEQ ID NO: 27 - DNA, 22 bp, Artificial Sequence
  Reverse primer
tccacagtcagcaatggtgatc

SEQ ID NO: 28 - DNA, 25 bp, Artificial Sequence
  Probe
tccaggaatggcaagaccagcaaga

SEQ ID NO: 29 - DNA, 15 bp, Artificial Sequence
  Forward primer
tcgccgcttgctgca

SEQ ID NO: 30 - DNA, 17 bp, Artificial Sequence
  Reverse primer
atcggccgtgatgtcga

SEQ ID NO: 31 - DNA, 23 bp, Artificial Sequence
  Probe
ccatggtcaaccccaccgtgttc

SEQ ID NO: 32 - DNA, 19 bp, Artificial Sequence
  Forward primer
cccaccgtgttcttcgaca

SEQ ID NO: 33 - DNA, 20 bp, Artificial Sequence
  Reverse primer
aaacagctcgaagcagacgc

SEQ ID NO: 34 - DNA, 19 bp, Artificial Sequence
  Probe
cacggctgatggcgagccc

SEQ ID NO: 35-37 - skipped (3 entries)

SEQ ID NO: 38 - DNA, 20 bp, Artificial Sequence
  Forward primer
ggcatccctgaggtcatgtc

SEQ ID NO: 39 - DNA, 19 bp, Artificial Sequence
  Reverse primer
ggctcacgcctttgctgtt

SEQ ID NO: 40 - DNA, 27 bp, Artificial Sequence
  Probe
cggctcgaggtagtgtttacagccctc

SEQ ID NO: 41 - DNA, 20 bp, Artificial Sequence
  Synthetic oligonucleotide
tcctggccccagagattcac

SEQ ID NO: 42 - DNA, 20 bp, Artificial Sequence
  Synthetic oligonucleotide
cagcagggtcttcctggccc

SEQ ID NO: 43 - DNA, 20 bp, Artificial Sequence
  Synthetic oligonucleotide
ggaacatgaggctcttccgg

SEQ ID NO: 44 - DNA, 20 bp, Artificial Sequence
  Synthetic oligonucleotide
tatgtatgtccgcccagccc

SEQ ID NO: 45 - DNA, 20 bp, Artificial Sequence
  Synthetic oligonucleotide
ccgtatatgtatgtccgccc

SEQ ID NO: 46 - DNA, 20 bp, Artificial Sequence
  Synthetic oligonucleotide
cgagccgttcagcctggagc

SEQ ID NO: 47 - DNA, 20 bp, Artificial Sequence
  Synthetic oligonucleotide
gcagtggtgtgtaagtggcc

SEQ ID NO: 48 - DNA, 20 bp, Artificial Sequence
  Synthetic oligonucleotide
gcagccagcatggttatcag

SEQ ID NO: 49 - DNA, 20 bp, Artificial Sequence
  Synthetic oligonucleotide
caggactgtggcagccagca

SEQ ID NO: 50 - DNA, 20 bp, Artificial Sequence
  Synthetic oligonucleotide
gcattgcccagcagggccag

SEQ ID NO: 51 - DNA, 20 bp, Artificial Sequence
  Synthetic oligonucleotide
gtgcctttggagcaggcatg

SEQ ID NO: 52 - DNA, 20 bp, Artificial Sequence
  Synthetic oligonucleotide
cgatgcctgcctcgtgcgag

SEQ ID NO: 53 - DNA, 20 bp, Artificial Sequence
  Synthetic oligonucleotide
tcgtggttcaacaccaggag

SEQ ID NO: 54 - DNA, 20 bp, Artificial Sequence
  Synthetic oligonucleotide
tctggatcaccttggcagtc

SEQ ID NO: 55 - DNA, 20 bp, Artificial Sequence
  Synthetic oligonucleotide
ggtagctggctcgctggaag

SEQ ID NO: 56 - DNA, 20 bp, Artificial Sequence
  Synthetic oligonucleotide
cttctcgcccgtgatatctg

SEQ ID NO: 57 - DNA, 20 bp, Artificial Sequence
  Synthetic oligonucleotide
gcatcatggccttctcgccc

SEQ ID NO: 58 - DNA, 20 bp, Artificial Sequence
  Synthetic oligonucleotide
atacttgacttggccaagga

SEQ ID NO: 59 - DNA, 20 bp, Artificial Sequence
  Synthetic oligonucleotide
atctggatgttgtgcaaccc

SEQ ID NO: 60 - DNA, 20 bp, Artificial Sequence
  Synthetic oligonucleotide
ctgctggcgatggacaagtg

SEQ ID NO: 61 - DNA, 20 bp, Artificial Sequence
  Synthetic oligonucleotide
cttccaccagctccacctgg

SEQ ID NO: 62 - DNA, 20 bp, Artificial Sequence
  Synthetic oligonucleotide
gagacatcaatggacttggc

SEQ ID NO: 63 - DNA, 20 bp, Artificial Sequence
  Synthetic oligonucleotide
ccacagacacgttctgaatg

SEQ ID NO: 64 - DNA, 20 bp, Artificial Sequence
  Synthetic oligonucleotide
gtggtgtagccatacttcag

SEQ ID NO: 65 - DNA, 20 bp, Artificial Sequence
  Synthetic oligonucleotide
tcaatacccagccaccaggc

SEQ ID NO: 66 - DNA, 20 bp, Artificial Sequence
  Synthetic oligonucleotide
tctcgaagtcaatggactga

SEQ ID NO: 67 - DNA, 20 bp, Artificial Sequence
  Synthetic oligonucleotide
gaggtcaatggcagagtcga

SEQ ID NO: 68 - DNA, 20 bp, Artificial Sequence
  Synthetic oligonucleotide
gtcagctgtgtgttgatctg

SEQ ID NO: 69 - DNA, 20 bp, Artificial Sequence
  Synthetic oligonucleotide
gcactctaccagagtcacag

SEQ ID NO: 70 - DNA, 20 bp, Artificial Sequence
  Synthetic oligonucleotide
agcagcttatggaaagacag

SEQ ID NO: 71 - DNA, 20 bp, Artificial Sequence
  Synthetic oligonucleotide
cgctccccttggagatgcag

SEQ ID NO: 72 - DNA, 20 bp, Artificial Sequence
  Synthetic oligonucleotide
gcttgatccacccaggctct

SEQ ID NO: 73 - DNA, 20 bp, Artificial Sequence
  Synthetic oligonucleotide
ccttcaggaccagcttcagg

SEQ ID NO: 74 - DNA, 20 bp, Artificial Sequence
  Synthetic oligonucleotide
gatctctttgcagatctgtc

SEQ ID NO: 75 - DNA, 20 bp, Artificial Sequence
  Synthetic oligonucleotide
atgatgttagagatgacgtt

SEQ ID NO: 76 - DNA, 20 bp, Artificial Sequence
  Synthetic oligonucleotide
ctggcagcccttgtctggac

SEQ ID NO: 77 - DNA, 20 bp, Artificial Sequence
  Synthetic oligonucleotide
tgtctccatctgaaaggatg

SEQ ID NO: 78 - DNA, 20 bp, Artificial Sequence
  Synthetic oligonucleotide
aggtaggaggctgtgatgac

SEQ ID NO: 79 - DNA, 20 bp, Artificial Sequence
  Synthetic oligonucleotide
tgaaatgacccttgtgatgg

SEQ ID NO: 80 - DNA, 20 bp, Artificial Sequence
  Synthetic oligonucleotide
ctctgagacattcttgtaga

SEQ ID NO: 81 - DNA, 20 bp, Artificial Sequence
  Synthetic oligonucleotide
accagaagtacagcatgcgg

SEQ ID NO: 82 - DNA, 20 bp, Artificial Sequence
  Synthetic oligonucleotide
gaaagctaccttggccagcg

SEQ ID NO: 83 - DNA, 20 bp, Artificial Sequence
  Synthetic oligonucleotide
cccatcaggctgagcatgag

SEQ ID NO: 84 - DNA, 20 bp, Artificial Sequence
  Synthetic oligonucleotide
gcactgccttgaactcgtct

SEQ ID NO: 85 - DNA, 20 bp, Artificial Sequence
  Synthetic oligonucleotide
gttgaagccccaggtctcca

SEQ ID NO: 86 - DNA, 20 bp, Artificial Sequence
  Synthetic oligonucleotide
cctcttggaagatttcctgg

SEQ ID NO: 87 - DNA, 20 bp, Artificial Sequence
  Synthetic oligonucleotide
agccgccgacaacctcttgg

SEQ ID NO: 88 - DNA, 20 bp, Artificial Sequence
  Synthetic oligonucleotide
ggcatcttgaggcagtggac

SEQ ID NO: 89 - DNA, 20 bp, Artificial Sequence
  Synthetic oligonucleotide
tgttttggcaggagatcttg

SEQ ID NO: 90 - DNA, 20 bp, Artificial Sequence
  Synthetic oligonucleotide
agaattgaccacgactccct

SEQ ID NO: 91 - DNA, 20 bp, Artificial Sequence
  Synthetic oligonucleotide
aggaatttcaccatcactga

SEQ ID NO: 92 - DNA, 20 bp, Artificial Sequence
  Synthetic oligonucleotide
acagaatgttgctggtctgg

SEQ ID NO: 93 - DNA, 20 bp, Artificial Sequence
  Synthetic oligonucleotide
cctcttcaaatgtgtaagct

SEQ ID NO: 94 - DNA, 20 bp, Artificial Sequence
  Synthetic oligonucleotide
ctggacggtagtcacgatat

SEQ ID NO: 95 - DNA, 20 bp, Artificial Sequence
  Synthetic oligonucleotide
ccaagaggcttaagaagagc

SEQ ID NO: 96 - DNA, 20 bp, Artificial Sequence
  Synthetic oligonucleotide
acagtctttggtgtaatctg

SEQ ID NO: 97 - DNA, 20 bp, Artificial Sequence
  Synthetic oligonucleotide
gctgctctcagtcaagttgg

SEQ ID NO: 98 - DNA, 20 bp, Artificial Sequence
  Synthetic oligonucleotide
gaagctctggatggactcgg

SEQ ID NO: 99 - DNA, 20 bp, Artificial Sequence
  Synthetic oligonucleotide
gcggtgatcattgactgcag

SEQ ID NO: 100 - DNA, 20 bp, Artificial Sequence
  Synthetic oligonucleotide
tgacctcagggatgcccaca

SEQ ID NO: 101 - DNA, 20 bp, Artificial Sequence
  Synthetic oligonucleotide
cactacctcgagccgagaca

SEQ ID NO: 102 - DNA, 20 bp, Artificial Sequence
  Synthetic oligonucleotide
cgcctttgctgttcatgagg

SEQ ID NO: 103 - DNA, 20 bp, Artificial Sequence
  Synthetic oligonucleotide
tgatgatgtcgaagaggctc

SEQ ID NO: 104 - DNA, 20 bp, Artificial Sequence
  Synthetic oligonucleotide
tctcgagtgataatctcagg

SEQ ID NO: 105 - DNA, 20 bp, Artificial Sequence
  Synthetic oligonucleotide
catctgcagcagcaggaagc

SEQ ID NO: 106 - DNA, 20 bp, Artificial Sequence
  Synthetic oligonucleotide
tccaccagcaggtgctcagg

SEQ ID NO: 107 - DNA, 20 bp, Artificial Sequence
  Synthetic oligonucleotide
tctagctcaagctctggagg

SEQ ID NO: 108 - DNA, 20 bp, Artificial Sequence
  Synthetic oligonucleotide
cccgacctccttggagactt

SEQ ID NO: 109 - DNA, 20 bp, Artificial Sequence
  Synthetic oligonucleotide
ttgccttctgctacaagccc

SEQ ID NO: 110 - DNA, 20 bp, Artificial Sequence
  Synthetic oligonucleotide
tccagctgtgagcctggtgc

SEQ ID NO: 111 - DNA, 20 bp, Artificial Sequence
  Synthetic oligonucleotide
acgctggaggagacaccagg

SEQ ID NO: 112 - DNA, 20 bp, Artificial Sequence
  Synthetic oligonucleotide
aacttccaccacgctggagg

SEQ ID NO: 113 - DNA, 20 bp, Artificial Sequence
  Synthetic oligonucleotide
catctccgtactcctaaccc

SEQ ID NO: 114 - DNA, 20 bp, Artificial Sequence
  Synthetic oligonucleotide
cagcactttaatgccagtgg

SEQ ID NO: 115 - DNA, 20 bp, Artificial Sequence
  Synthetic oligonucleotide
gacaagtggctgatctggat

SEQ ID NO: 116 - DNA, 20 bp, Artificial Sequence
  Synthetic oligonucleotide
gcttgccttctgctacaagc

SEQ ID NO: 117 - DNA, 20 bp, Artificial Sequence
  Synthetic oligonucleotide
ccagtgggcctttaggatag

SEQ ID NO: 118 - DNA, 24 bp, Artificial Sequence
  Forward primer
cagctgacctgtgactctggtaga

SEQ ID NO: 119 - DNA, 24 bp, Artificial Sequence
  Reverse primer
cagcttatggaaagacaggtagca

SEQ ID NO: 120 - DNA, 19 bp, Artificial Sequence
  Probe
tgcggaccgatgcccctga

SEQ ID NO: 121 - DNA, 20 bp, Artificial Sequence
  Synthetic oligonucleotide
ccttccctgaaggttcctcc

SEQ ID NO: 122 - DNA, 21 bp, Artificial Sequence
  Forward primer
ggacttggtaggacggaacct

SEQ ID NO: 123 - DNA, 23 bp, Artificial Sequence
  Reverse primer
atcctcatcctcgtcattcaaag

SEQ ID NO: 124 - DNA, 24 bp, Artificial Sequence
  Probe
aggcccagacctgtaaaggcgaag

SEQ ID NO: 125 - DNA, 23 bp, Artificial Sequence
  Forward primer
actctgggttcaaccgttagtca

SEQ ID NO: 126 - DNA, 22 bp, Artificial Sequence
  Reverse primer
tatcccagaagtcccgagtcaa

SEQ ID NO: 127 - DNA, 20 bp, Artificial Sequence
  Probe
ctgcaggaacggctgggccc

SEQ ID NO: 128 - DNA, 20 bp, Artificial Sequence
  Forward primer
tgacaacgacaccgtgtcct

SEQ ID NO: 129 - DNA, 19 bp, Artificial Sequence
  Reverse primer
atgcgacttgtcaggctgg

SEQ ID NO: 130 - DNA, 23 bp, Artificial Sequence
  Probe
cgtggagaaccgcagcctccatt

(19) United States
(12) Patent Application Publication
(10) Pub. No.: US 2013/0053430 A1
(43) Pub. Date: Feb. 28, 2013

(54) MODULATION OF CETP EXPRESSION

(71) Applicants: Thomas A. Bell, III, Encinitas, CA (US); Rosanne M. Crooke, Carlsbad, CA (US); Mark J. Graham, San Clemente, CA (US)

(72) Inventors: Thomas A. Bell, III, Encinitas, CA (US); Rosanne M. Crooke, Carlsbad, CA (US); Mark J. Graham, San Clemente, CA (US)

(73) Assignee: Isis Pharmaceuticals, Inc., Carlsbad, CA (US)

(21) Appl. No.: 13/639,837

(22) PCT Filed: Apr. 7, 2011
(86) PCT No.: PCT/US11/31611

§ 371 Date: Oct. 5, 2012

Related U.S. Application Data
(60) Provisional application No. 61/321,783, filed on Apr. 7, 2010.

(51) Int. Cl. A61K 31/7125; A61K 31/7088; A61K 31/712; A61K 31/7115; A61P 3/00; A61P 3/06; A61P 9/00; A61P 9/12; A61P 3/04; A61P 3/10; C12N 15/113; A61K 31/713

(52) U.S. Cl. 514/44 A; 536/24.5

(57) ABSTRACT

Provided herein are methods, compounds, and compositions for reducing expression of a CETP mRNA and protein in an animal. Also provided herein are methods, compounds, and compositions for increasing HDL levels and/or HDL activity and reducing plasma lipids, plasma glucose and atherosclerotic plaques in an animal. Such methods, compounds, and compositions are useful to treat, prevent, delay, or ameliorate any one or more of cardiovascular disease or metabolic disease, or a symptom thereof.

### SEQUENCE LISTING

The present application is being filed along with a Sequence Listing in electronic format. The Sequence Listing is provided as a file entitled 20110407_BIOL0131WOSEQ.txt, created on Apr. 7, 2011 which is 64 KB in size. The information in the electronic format of the sequence listing is incorporated herein by reference in its entirety.

### FIELD OF THE INVENTION

Provided herein are methods, compounds, and compositions for reducing expression of gp130 mRNA and protein in an animal. Also, provided herein are methods, compounds, and compositions having a gp130 inhibitor for reducing gp130 related diseases or conditions in an animal. Such methods, compounds, and compositions are useful, for example, to treat, prevent, delay or ameliorate any one or more of cardiovascular disease or inflammatory syndrome, or a symptom thereof, in an animal.

### BACKGROUND OF THE INVENTION

Control of the risk factors involved in hypercholesterolemia and cardiovascular disease has been the focus of much research in academia and industry. Because an elevated level of circulating plasma low density lipoprotein (LDL) cholesterol has been identified as an independent risk factor in the development of hypercholesterolemia and cardiovascular disease, many strategies have been directed at lowering the levels of cholesterol carried in this atherogenic lipoprotein. In contrast, high density lipoprotein (HDL) plays a crucial role in the protection of blood vessels from atherosclerosis by delivering excess cholesterol from peripheral tissues to the liver for excretion into bile in a process known as reverse cholesterol transport (Yamashita et al., Biochim. Biophys. Acta, 2000, 1529, 257-275). After cholesterol from peripheral cells is taken up by HDL, it is converted into cholesteroyl ester. This HDL cholesteryl ester can either be trafficked back to and taken up by the liver or transferred to very low density lipoprotein (VLDL), intermediate density lipoprotein (IDL) and low density lipoprotein (LDL) (Yamashita et al., Biochim. Biophys. Acta, 2000, 1529, 257-275).

The redistribution of HDL cholesteryl ester is mediated by cholesteryl ester transfer protein (also known as CETP and lipid transfer protein II). During this process CETP facilitates the neutral lipid transfer of cholestery ester from HDL for triglyceride from VLDL, IDL and LDL (Yamashita et al., Biochim. Biophys. Acta, 2000, 1529, 257-275). Cholesteryl ester transfer protein exists in humans and rabbits but not in rodents (Hirano et al., Curr. Opin. Lipidol., 2000, 11, 589-596).

Cholesteryl ester transfer protein was cloned in 1987 (Drayna et al., Nature, 1987, 327, 632-634), and subsequently mapped to chromosome 16q21 (Lusis et al., Genomics, 1987, 1, 232-235). It is expressed in the liver, spleen, small intestine, adipose tissue, adrenal gland, kidney, heart and skeletal muscle and is secreted by a variety of cell types including monocyte-derived macrophages, B-lymphocytes, adipocytes and hepatocytes (Yamashita et al., Biochim. Biophys. Acta, 2000, 1529, 257-275).

Two isoforms of cholesteryl ester transfer protein mRNA are known: a full length form and an alternatively spliced form in which exon 9 is deleted (Inazu et al., Biochemistry, 1992, 31, 2352-2358). The alternatively spliced form has been demonstrated to express a protein which is inactive in lipid transfer and its expression has been suggested to serve as a switch for modulation of lipid transfer activity within specific tissues (Inazu et al., Biochemistry, 1992, 31, 2352-2358).

A number of transgenic animals, including rodents, containing the human cholesteryl ester transfer protein gene have been engineered and reviewed (Barter et. al., ATVB, 2003, 23, 160-167). The relationships between cholesteryl ester transfer protein overexpression and atherosclerosis have proven to be very complex in mouse models where lipoprotein metabolism is quite different from that of humans (de Grooth et. al., Journal of Lipid Res, 2004, 45, 1967-1974). In mouse models where liver-mediated uptake of atherogenic lipoproteins is compromised, CETP activity is proatherogenic but in the presence of hypertriglyceridemia and dysfunctional HDL, CETP activity can be beneficial (de Grooth et. al., Journal of Lipid Res, 2004, 45, 1967-1974).

Deficiency of cholesteryl ester transfer protein, arising from either a splicing defect or a missense mutation, causes various abnormalities in the concentration, composition and function of cholesteryl ester transfer protein and has been identified as the most frequent cause of hyperalphalipoproteinemia (HALP) in Asian populations (Yamashita et al., Atherosclerosis, 2000, 152, 271-285).

The effects of cholesteryl ester transfer protein activity on reverse cholesterol transport have been examined in a review of reverse cholesterol transport in diabetes mellitus (Quintao et al., Diabetes Metab. Res. Rev., 2000, 16, 237-250).

The process of transfer of cholesteryl ester from HDL to LDL and VLDL may be detrimental because LDL and VLDL are known to be atherogenic (Chong and Bachenheimer, Drugs, 2000, 60, 55-93). Support for this hypothesis may be drawn from the absence of cholesteryl ester transfer protein in rats which exhibit a very low incidence of atherosclerosis (Chong and Bachenheimer, Drugs, 2000, 60, 55-93) and from observations that Japanese subjects with cholesteryl ester transfer protein deficiency have high HDL levels and an increased life span (Inazu et al., N Engl. J. Med., 1990, 323, 1234-1238). In contrast, another study of Japanese men with reduced cholesteryl ester transfer protein levels found an increased risk for coronary heart disease (Ishigami et al., J. Biochem. (Tokyo), 1994, 116, 257-262).

Small molecule inhibitors of cholesteryl ester transfer protein are well represented in the art and include a variety of structural classes including sterols, polycyclic natural products and heterocycles (Sikorski and Glenn, Annu. Rep. Med. Chem., 2000, 35, 251-260). Antibodies to cholesteryl ester transfer protein (Saito et al., J. Lipid Res., 1999, 40, 2013-2021; Sugano et al., J. Lipid Res., 2000, 41, 126-133) and peptides from hog plasma (Cho et al., Biochim. Biophys. Acta, 1998, 1391, 133-144) have also been demonstrated to act as inhibitors of the human cholesteryl ester transfer protein.

The small molecule inhibitors (SMIs) of CETP have been extensively tested in the clinic and the results from these trials have yielded a wide range of effects on pharmacology, efficacy, and tolerability (Vergeer and Stroes, Am J Cardiol., 2009, 104, 32E-8E). One of the initial CETP SMIs in development was Torcetrapib. In early clinical trials Torcetrapib was found to be a potent inhibitor of CETP and provided beneficial shifts in the LDL/HDL ratio (van der Steeg W A et al., Curr Opin Lipidol., 2004, 15(6), 631-6). However, the development of Torcetrapib was halted in Phase III due to an increase in adverse events in the treatment groups. The negative of effects of Torcetrapib were attributed to increases in blood pressure and circulating aldosterone levels (Joy and Hegele, Curr Opin Cardiol., 2009, 24(4), 364-71). Furthermore, patients treated with Torcetrapib have also presented enlarged, apoAII enriched HDL, that potentially have delayed catabolism (Brousseau M E et al., J Lipid Res., 2009, 50(7), 1456-62). The two other CETP SMIs in clinical development, Anacetrapib and Dalcetrapib (formerly JTT-705), have not shown negative effects on blood pressure and aldosterone and provide beneficial shifts in HDL and LDL (Masson D, Curr Opin Investig Drugs, 2009, 10(9), 980-7 and Rennings and Stalenhoef, Expert Opin Investig Drugs, 2008, 17(10), 1589-97). However, effects of these compounds have on HDL metabolism and clearance has not been described in detail at this time. The upcoming results from Phase III trials will Anacetrapib and Dalcetrapib will show if the small molecule inhibition of CETP is a viable therapeutic strategy for cardiovascular disease.

A phosphorothioate oligonucleotide targeting human cholesteryl ester transfer protein nucleotides 329 to 349 was used to inhibit expression of human cholesteryl ester transfer protein in a human cholesteryl ester transfer protein-transfected Chinese hamster ovary (CHO) cell line (Liu et al., Arterioscler. Thromb. Vase. Biol., 1999, 19, 2207-2213). In this study, a system for targeted delivery of the antisense phosphorothioate oligonucleotides to the liver was developed through conjugation of the oligonucleotide to N,N-dipalmitylglycyl-apolipoprotein E (129-169) peptide which acts as an LDL receptor ligand (Liu et al., Arterioscler. Thromb. Vasc. Biol., 1999, 19, 2207-2213).

A 21-mer antisense oligonucleotide targeting positions 148 to 168 of the rabbit cholesteryl ester transfer protein sequence coupled to an asialoglycoprotein-poly-L-lysine carrier molecule was employed to decrease expression of cholesteryl ester transfer protein in in vivo studies of the effects of cholesteryl ester transfer protein on plasma lipoprotein cholesterol levels (Sugano and Makino, J. Biol. Chem., 1996, 271, 19080-19083) and on the development of atherosclerosis in rabbits (Sugano et al., J. Biol. Chem., 1998, 273, 5033-5036). These investigations conclude that administration of the antisense oligonucleotide-asialoglycoprotein conjugate to rabbits can lower plasma levels of LDL and VLDL (Sugano and Makino, J. Biol. Chem., 1996, 271, 19080-19083) and suppress the incidence of atherosclerosis in rabbits (Sugano et al., J. Biol. Chem., 1998, 273, 5033-5036).

Disclosed and claimed in German patent DE 19731609 are antisense oligonucleotides (including modified DNA/RNA hybrid oligonucleotides) to human cholesteryl ester transfer protein for the purpose of inhibition of expression of cholesteryl ester transfer protein (Budzinksi et al., 1999). Additionally disclosed and claimed in the same patent are transcription constructs of the human cholesteryl ester transfer protein gene containing 5′-non-translating regions with regulatory sequences (Budzinksi et al., 1999).

Disclosed in U.S. patent application Ser. Nos. 09/925,139, 11/031,827 and international application PCT/US02/24919 are antisense oligonucleotides targeting CETP and methods of modulating CETP.

Currently, inhibitors of cholesteryl ester transfer protein include several classes of small molecules, antibodies, peptides and the previously cited examples of antisense inhibitors. Of all these inhibitors, the small molecule inhibitors of CETP have been tested the most extensively in the clinic and in the lab and have shown to provide positive effects on overall plasma lipoprotein distribution (Masson D, Curr Opin Investig Drugs, 2009, 10(9), 980-7 and Rennings and Stalenhoef, Expert Opin Investig Drugs, 2008, 17(10), 1589-97). However, due to the potential negative effects on blood pressure and aldosterone coupled with potentially negative alterations in HDL subspecies observed in patients treated with small molecule inhibitors, there still is a need for therapeutic agents that inhibit CETP activity by an alternative means (Joy and Hegele, Curr Opin Cardiol., 2009, 24(4), 364-71 and Brousseau M E et al., J Lipid Res., 2009, 50(7), 1456-62). Antisense inhibition of CETP provides a unique advantage over traditional small molecule inhibitors in that antisense inhibitors do not rely on competitive binding of the compound to the protein and inhibit activity directly by reducing the expression of CETP.

Antisense compounds readily accumulate in the tissues where CETP is expressed such as liver and adipose tissue (Antisense Drug Technology 2^{nd} Edition, S T Crooke, Ed., CRC Press, Boca Raton, Fla.) making antisense technology uniquely suited to target CETP expression and function. Antisense technology is emerging as an effective means for reducing the expression of certain gene products and may therefore prove to be uniquely useful in a number of therapeutic, diagnostic, and research applications for the modulation of CETP.

There is a currently a lack of acceptable options for treating cardiovascular and metabolic disorders. It is therefore an object herein to provide compounds and methods for the treatment of such diseases and disorder.

All documents, or portions of documents, cited in this application, including, but not limited to, patents, patent applications, articles, books, and treatises, are hereby expressly incorporated-by-reference for the portions of the document discussed herein, as well as in their entirety.

### SUMMARY OF THE INVENTION

Provided herein are antisense compounds useful for modulating gene expression and associated pathways via antisense mechanisms of action such as RNaseH, RNAi and dsRNA enzymes, as well as other antisense mechanisms based on target degradation or target occupancy.

Provided herein are methods, compounds, and compositions for inhibiting expression of CETP and treating, preventing, delaying or ameliorating a CETP related disease, condition or a symptom thereof. In certain embodiments, the CETP related disease or condition is cardiovascular disease or inflammatory disease.

In certain embodiments, the compounds or compositions of the invention comprise a modified oligonucleotide 10 to 30 linked nucleosides in length targeted to CETP. The CETP target can have a sequence selected from any one of SEQ ID NOs: 1-4. The modified oligonucleotide targeting CETP can have a nucleobase sequence comprising at least 8 contiguous nucleobases complementary to an equal length portion of SEQ ID NOs: 1-4. The modified oligonucleotide can have a nucleobase sequence comprising at least 8, 9, 10, 11, 12, 13, 14, 15, 16, 17, 18, 19, 20, 21, 22, 23, 24, 25, 26, 27, 28, 29 or 30 contiguous nucleobases. The contiguous nucleobase portion of the modified oligonucleotide can be complementary to an equal length portion of a CETP region selected from any one of SEQ ID NOs: 1-4.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, the modified oligonucleotide consists of 20 linked nucleosides, the gap segment consisting of ten linked deoxynucleosides, the 5′ wing segment consisting of five linked nucleosides, the 3′ wing segment consisting of five linked nucleosides, each nucleoside of each wing segment comprises a 2′-O-methoxyethyl sugar, each internucleoside linkage is a phosphorothioate linkage and each cytosine is a 5-methylcytosine.

Certain embodiments provide a method of reducing CETP expression or activity in an animal comprising administering to the animal a compound comprising the modified oligonucleotide targeting CETP described herein.

Certain embodiments provide a method of increasing HDL levels and/or HDL activity in an animal comprising administering to the animal a compound comprising the modified oligonucleotide targeting CETP described herein.

Certain embodiments provide a method of reducing LDL, TG or glucose levels in an animal comprising administering to the animal a compound comprising the modified oligonucleotide targeted to CETP described herein.

Certain embodiments provide a method of reducing the LDL/HDL ratio in an animal comprising administering to the animal a compound comprising the modified oligonucleotide targeted to CETP described herein.

Certain embodiments provide a method of ameliorating cardiovascular disease or metabolic disease in an animal comprising administering to the animal a compound comprising a modified oligonucleotide targeted to CETP described herein.

Certain embodiments provide a method for treating an animal with cardiovascular disease or metabolic disease comprising: 1) identifying the animal with cardiovascular disease or metabolic disease, and 2) administering to the animal a therapeutically effective amount of a compound comprising a modified oligonucleotide consisting of 20 linked nucleosides and having a nucleobase sequence at least 90% complementary to SEQ ID NO: 1-4 as measured over the entirety of said modified oligonucleotide, thereby treating the animal with cardiovascular disease or metabolic disease. In certain embodiments, the therapeutically effective amount of the compound administered to the animal reduces cardiovascular disease or metabolic disease in the animal.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques can be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5′ position. A 5-methylcytosine is a modified nucleobase.

“About” means within ±10% of a value. For example, if it is stated, “the compounds affected at least about 70% inhibition of CETP”, it is implied that the CETP levels are inhibited within a range of 63% and 77%.

“Active pharmaceutical agent” means the substance or substances in a pharmaceutical composition that provide a therapeutic benefit when administered to an individual. For example, in certain embodiments an antisense oligonucleotide targeted to CETP is an active pharmaceutical agent.

“Active target region” or “target region” means a region to which one or more active antisense compounds is targeted. “Active antisense compounds” means antisense compounds that reduce target nucleic acid levels or protein levels.

“Adipogenesis” means the development of fat cells from preadipocytes. “Lipogenesis” means the production or formation of fat, either fatty degeneration or fatty infiltration.

“Adiposity” or “Obesity” refers to the state of being obese or an excessively high amount of body fat or adipose tissue in relation to lean body mass. The amount of body fat includes concern for both the distribution of fat throughout the body and the size and mass of the adipose tissue deposits. Body fat distribution can be estimated by skin-fold measures, waist-to-hip circumference ratios, or techniques such as ultrasound, computed tomography, or magnetic resonance imaging. According to the Center for Disease Control and Prevention, individuals with a body mass index (BMI) of 30 or more are considered obese. The term “Obesity” as used herein includes conditions where there is an increase in body fat beyond the physical requirement as a result of excess accumulation of adipose tissue in the body. The term “obesity” includes, but is not limited to, the following conditions: adult-onset obesity; alimentary obesity; endogenous or inflammatory obesity; endocrine obesity; familial obesity; hyperinsulinar obesity; hyperplastic-hypertrophic obesity; hypogonadal obesity; hypothyroid obesity; lifelong obesity; morbid obesity and exogenous obesity.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing an agent to an animal, and includes, but is not limited to, administering by a medical professional and self-administering.

“Agent” means an active substance that can provide a therapeutic benefit when administered to an animal. “First Agent” means a therapeutic compound of the invention. For example, a first agent can be an antisense oligonucleotide targeting CETP. “Second agent” means a second therapeutic compound of the invention (e.g. a second antisense oligonucleotide targeting CETP) and/or a non-CETP therapeutic compound.

“Amelioration” refers to a lessening of at least one indicator, sign, or symptom of an associated disease, disorder, or condition. The severity of indicators can be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. As used herein, the term “antisense compound” encompasses pharmaceutically acceptable derivatives of the compounds described herein.

“Antisense inhibition” means the reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid. As used herein, the term “antisense oligonucleotide” encompasses pharmaceutically acceptable derivatives of the compounds described herein.

“ApoB-containing lipoprotein” means any lipoprotein that has apolipoprotein B as its protein component, and is understood to include LDL, VLDL, IDL, and lipoprotein(a) and can be generally targeted by lipid lowering agent and therapies. “ApoB-100-containing LDL” means ApoB-100 isoform containing LDL.

“Atherosclerosis” means a hardening of the arteries affecting large and medium-sized arteries and is characterized by the presence of fatty deposits. The fatty deposits are called “atheromas” or “plaques,” which consist mainly of cholesterol and other fats, calcium and scar tissue, and damage the lining of arteries.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Cardiovascular disease” or “cardiovascular disorder” refers to a group of conditions related to the heart, blood vessels, or the circulation. Examples of cardiovascular diseases include, but are not limited to, aneurysm, angina, arrhythmia, atherosclerosis, cerebrovascular disease (stroke), coronary heart disease, hypertension, dyslipidemia, hyperlipidemia, and hypercholesterolemia.

“Cholesteryl ester transfer protein” or “CETP” (also known as lipid transfer protein II) means any nucleic acid or protein of CETP.

“CETP expression” means the level of mRNA transcribed from the gene encoding CETP or the level of protein translated from the mRNA. CETP expression can be determined by art known methods such as a Northern or Western blot.

“CETP nucleic acid” means any nucleic acid encoding CETP. For example, in certain embodiments, a CETP nucleic acid includes a DNA sequence encoding CETP, a RNA sequence transcribed from DNA encoding CETP (including genomic DNA comprising introns and exons), and a mRNA sequence encoding CETP. “CETP mRNA” means a mRNA encoding a CETP protein.

“Chemically distinct region” refers to a region of an antisense compound that is in some way chemically different than another region of the same antisense compound. For example, a region having 2% O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses parallel or sequential administration.

“Constrained ethyl” or “cEt” refers to a bicyclic nucleoside having a furanosyl sugar that comprises a methyl(methyleneoxy) (4′-CH(CH₃)—O-2′) bridge between the 4′ and the 2′ carbon atoms.

“Cholesterol” is a sterol molecule found in the cell membranes of all animal tissues. Cholesterol must be transported in an animal's blood plasma by lipoproteins including very low density lipoprotein (VLDL), intermediate density lipoprotein (IDL), low density lipoprotein (LDL), and high density lipoprotein (HDL). “Plasma cholesterol” refers to the sum of all lipoproteins (VDL, IDL, LDL, HDL) esterified and/or non-estrified cholesterol present in the plasma or serum.

“Cholesterol absorption inhibitor” means an agent that inhibits the absorption of exogenous cholesterol obtained from diet.

“Complementarity” means the capacity for pairing between nucleobases of a first nucleic acid and a second nucleic acid. In certain embodiments, complementarity between the first and second nucleic acid may be between two DNA strands, between two RNA strands, or between a DNA and an RNA strand. In certain embodiments, some of the nucleobases on one strand are matched to a complementary hydrogen bonding base on the other strand. In certain embodiments, all of the nucleobases on one strand are matched to a complementary hydrogen bonding base on the other strand. In certain embodiments, a first nucleic acid is an antisense compound and a second nucleic acid is a target nucleic acid. In certain such embodiments, an antisense oligonucleotide is a first nucleic acid and a target nucleic acid is a second nucleic acid.

“Contiguous nucleobases” means nucleobases immediately adjacent to each other.

“Cross-reactive” means an oligomeric compound targeting one nucleic acid sequence can hybridize to a different nucleic acid sequence. For example, in some instances an antisense oligonucleotide targeting human CETP can cross-react with a murine CETP. Whether an oligomeric compound cross-reacts with a nucleic acid sequence other than its designated target depends on the degree of complementarity the compound has with the non-target nucleic acid sequence.

“Cure” means a method that restores health or a prescribed treatment for an illness.

“Coronary heart disease (CHD)” means a narrowing of the small blood vessels that supply blood and oxygen to the heart, which is often a result of atherosclerosis.

“Deoxyribonucleotide” means a nucleotide having a hydrogen at the 2′ position of the sugar portion of the nucleotide. Deoxyribonucleotides may be modified with any of a variety of substituents.

“Diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

“Diabetic dyslipidemia” or “type 2 diabetes with dyslipidemia” means a condition characterized by Type 2 diabetes, reduced HDL-C, elevated triglycerides, and elevated small, dense LDL particles.

“Diluent” means an ingredient in a composition that lacks pharmacological activity, but is pharmaceutically necessary or desirable. For example, the diluent in an injected composition can be a liquid, e.g. saline solution.

“Dyslipidemia” refers to a disorder of lipid and/or lipoprotein metabolism, including lipid and/or lipoprotein overproduction or deficiency. Dyslipidemias may be manifested by elevation of lipids such as cholesterol and triglycerides as well as lipoproteins such as low-density lipoprotein (LDL) cholesterol.

“Dosage unit” means a form in which a pharmaceutical agent is provided, e.g. pill, tablet, or other dosage unit known in the art. In certain embodiments, a dosage unit is a vial containing lyophilized antisense oligonucleotide. In certain embodiments, a dosage unit is a vial containing reconstituted antisense oligonucleotide.

“Dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month. Doses can be expressed, for example, as mg/kg.

“Effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense compound and a second nucleic acid is a target nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.”

“Gap-widened” means a chimeric antisense compound having a gap segment of 12 or more contiguous 2′-deoxyribonucleosides positioned between and immediately adjacent to 5′ and 3′ wing segments having from one to six nucleosides.

“Glucose” is a monosaccharide used by cells as a source of energy and inflammatory intermediate. “Plasma glucose” refers to glucose present in the plasma.

“High density lipoprotein-C(HDL-C)” means cholesterol associated with high density lipoprotein particles. Concentration of HDL-C in serum (or plasma) is typically quantified in mg/dL or nmol/L. “HDL-C” and “plasma HDL-C” mean HDL-C in serum and plasma, respectively.

“HMG-CoA reductase inhibitor” means an agent that acts through the inhibition of the enzyme HMG-CoA reductase, such as atorvastatin, rosuvastatin, fluvastatin, lovastatin, pravastatin, and simvastatin.

“Hybridization” means the annealing of complementary nucleic acid molecules. In certain embodiments, complementary nucleic acid molecules include an antisense compound and a target nucleic acid.

“Hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. (1988) 148, 36-39).

“Hyperlipidemia” or “hyperlipemia” is a condition characterized by elevated serum lipids or circulating (plasma) lipids. This condition manifests an abnormally high concentration of fats. The lipid fractions in the circulating blood are cholesterol, low density lipoproteins, very low density lipoproteins and triglycerides.

“Hypertriglyceridemia” means a condition characterized by elevated triglyceride levels.

“Identifying” or “selecting an animal with metabolic or cardiovascular disease” means identifying or selecting a subject having been diagnosed with a metabolic disease, a cardiovascular disease, or a metabolic syndrome; or, identifying or selecting a subject having any symptom of a metabolic disease, cardiovascular disease, or metabolic syndrome including, but not limited to, hypercholesterolemia, hyperglycemia, hyperlipidemia, hypertriglyceridemia, hypertension increased insulin resistance, decreased insulin sensitivity, above normal body weight, and/or above normal body fat content or any combination thereof. Such identification may be accomplished by any method, including but not limited to, standard clinical tests or assessments, such as measuring serum or circulating (plasma) cholesterol, measuring serum or circulating (plasma) blood-glucose, measuring serum or circulating (plasma) triglycerides, measuring blood-pressure, measuring body fat content, measuring body weight, and the like.

“Improved cardiovascular outcome” means a reduction in the occurrence of adverse cardiovascular events, or the risk thereof. Examples of adverse cardiovascular events include, without limitation, death, reinfarction, stroke, cardiogenic shock, pulmonary edema, cardiac arrest, and atrial dysrhythmia.

“Immediately adjacent” means there are no intervening elements between the immediately adjacent elements, for example, between regions, segments, nucleotides and/or nucleosides.

“Individual” or “subject” or “animal” means a human or non-human animal selected for treatment or therapy.

“Induce”, “inhibit”, “potentiate”, “elevate”, “increase”, “decrease” or the like, e.g. denote quantitative differences between two states. For example, “an amount effective to inhibit the activity or expression of CETP” means that the level of activity or expression of CETP in a treated sample will differ from the level of CETP activity or expression in an untreated sample. Such terms are applied to, for example, levels of expression, and levels of activity.

“Inhibiting the expression or activity” refers to a reduction or blockade of the expression or activity of a RNA or protein and does not necessarily indicate a total elimination of expression or activity.

“Insulin resistance” is defined as the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake whereas insulin resistance in liver reduces glucose storage, with both effects serving to elevate blood glucose. High plasma levels of insulin and glucose due to insulin resistance often leads to metabolic syndrome and type 2 diabetes.

“Insulin sensitivity” is a measure of how effectively an individual processes glucose. An individual having high insulin sensitivity effectively processes glucose whereas an individual with low insulin sensitivity does not effectively process glucose.

“Internucleoside linkage” refers to the chemical bond between nucleosides.

“Intravenous administration” means administration into a vein.

“Linked nucleosides” means adjacent nucleosides which are bonded together.

“Lipid-lowering” means a reduction in one or more lipids in a subject. Lipid-lowering can occur with one or more doses over time.

“Lipid-lowering therapy” or “lipid lowering agent” means a therapeutic regimen provided to a subject to reduce one or more lipids in a subject. In certain embodiments, a lipid-lowering therapy is provided to reduce one or more of CETP, ApoB, total cholesterol, LDL-C, VLDL-C, non-HDL-C, triglycerides, small dense LDL particles, and Lp(a) in a subject. Examples of lipid-lowering therapy include statins, fibrates, MTP inhibitors.

“Lipoprotein”, such as VLDL, LDL and HDL, refers to a group of proteins found in the serum, plasma and lymph and are important for lipid transport. The chemical composition of each lipoprotein differs in that the HDL has a higher proportion of protein versus lipid, whereas the VLDL has a lower proportion of protein versus lipid.

“Low density lipoprotein-cholesterol (LDL-C)” means cholesterol carried in low density lipoprotein particles. Concentration of LDL-C in serum (or plasma) is typically quantified in mg/dL or nmol/L. “Serum LDL-C” and “plasma LDL-C” mean LDL-C in the serum and plasma, respectively.

“Major risk factors” refers to factors that contribute to a high risk for a particular disease or condition. In certain embodiments, major risk factors for coronary heart disease include, without limitation, cigarette smoking, hypertension, low HDL-C, family history of coronary heart disease, age, and other factors disclosed herein.

“Metabolic disorder” or “metabolic disease” refers to a condition characterized by an alteration or disturbance in metabolic function. “Metabolic” and “metabolism” are terms well known in the art and generally include the whole range of biochemical processes that occur within a living organism. Metabolic disorders include, but are not limited to, hyperglycemia, prediabetes, diabetes (type I and type 2), obesity, insulin resistance, metabolic syndrome and dyslipidemia due to type 2 diabetes.

“Metabolic syndrome” means a condition characterized by a clustering of lipid and non-lipid cardiovascular risk factors of metabolic origin. In certain embodiments, metabolic syndrome is identified by the presence of any 3 of the following factors: waist circumference of greater than 102 cm in men or greater than 88 cm in women; serum triglyceride of at least 150 mg/dL; HDL-C less than 40 mg/dL in men or less than 50 mg/dL in women; blood pressure of at least 130/85 mmHg; and fasting glucose of at least 110 mg/dL. These determinants can be readily measured in clinical practice (JAMA, 2001, 285: 2486-2497).

“Mismatch” or “non-complementary nucleobase” refers to the case when a nucleobase of a first nucleic acid is not capable of pairing with the corresponding nucleobase of a second or target nucleic acid.

“Mixed dyslipidemia” means a condition characterized by elevated cholesterol and elevated triglycerides.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleoside” means a nucleoside having, independently, a modified sugar moiety or modified nucleobase.

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising at least one modified nucleotide.

“Modified sugar” refers to a substitution or change from a natural sugar.

“Motif” means the pattern of chemically distinct regions in an antisense compound.

“MTP inhibitor” means an agent inhibits the enzyme, microsomal triglyceride transfer protein.

“Naturally occurring internucleoside linkage” means a 3′ to 5′ phosphodiester linkage.

“Natural sugar moiety” means a sugar found in DNA (2′-H) or RNA (2′-OH).

“Non-alcoholic fatty liver disease” or “NAFLD” means a condition characterized by fatty inflammation of the liver that is not due to excessive alcohol use (for example, alcohol consumption of over 20 g/day). In certain embodiments, NAFLD is related to insulin resistance and the metabolic syndrome. NAFLD encompasses a disease spectrum ranging from simple triglyceride accumulation in hepatocytes (hepatic steatosis) to hepatic steatosis with inflammation (steatohepatitis), fibrosis, and cirrhosis.

“Nonalcoholic steatohepatitis” (NASH) occurs from progression of NAFLD beyond deposition of triglycerides. A “second hit” capable of inducing necrosis, inflammation, and fibrosis is required for development of NASH. Candidates for the second-hit can be grouped into broad categories: factors causing an increase in oxidative stress and factors promoting expression of proinflammatory cytokines

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA). A nucleic acid can also comprise a combination of these elements in a single molecule.

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the oligonucleotide and the target nucleic acid are considered to be complementary at that nucleobase pair.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base, and not necessarily the linkage at one or more positions of an oligomeric compound; for example nucleoside mimetics having morpholino, cyclohexenyl, cyclohexyl, tetrahydropyranyl, bicyclo or tricyclo sugar mimetics such as non furanose sugar units.

“Nucleotide” means a nucleoside having a phosphate group covalently linked to the sugar portion of the nucleoside.

“Nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

“Oligomeric compound” or “oligomer” refers to a polymeric structure comprising two or more sub-structures and capable of hybridizing to a region of a nucleic acid molecule. In certain embodiments, oligomeric compounds are oligonucleosides. In certain embodiments, oligomeric compounds are oligonucleotides. In certain embodiments, oligomeric compounds are antisense compounds. In certain embodiments, oligomeric compounds are antisense oligonucleotides. In certain embodiments, oligomeric compounds are chimeric oligonucleotides.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration. Administration can be continuous, or chronic, or short or intermittent.

“Peptide” means a molecule formed by linking at least two amino acids by amide bonds. Peptide refers to polypeptides and proteins.

“Pharmaceutical agent” means a substance that provides a therapeutic benefit when administered to an individual. For example, in certain embodiments, an antisense oligonucleotide targeted to CETP is pharmaceutical agent.

“Pharmaceutical composition” or “composition” means a mixture of substances suitable for administering to an individual. For example, a pharmaceutical composition can comprise one or more active agents and a sterile aqueous solution.

“Pharmaceutically acceptable carrier” means a medium or diluent that does not interfere with the structure of the oligonucleotide. Certain, of such carries enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection, infusion or topical administration. For example, a pharmaceutically acceptable carrier can be a sterile aqueous solution.

“Pharmaceutically acceptable derivative” encompasses derivatives of the compounds described herein such as solvates, hydrates, esters, prodrugs, polymorphs, isomers, isotopically labelled variants, conjugates, pharmaceutically acceptable salts and other derivatives known in the art.

“Pharmaceutically acceptable salts” or “salts” means physiologically and pharmaceutically acceptable salts of antisense compounds, i.e., salts that retain the desired biological activity of the parent oligonucleotide and do not impart undesired toxicological effects thereto. The term “pharmaceutically acceptable salt” or “salt” includes a salt prepared from pharmaceutically acceptable non-toxic acids or bases, including inorganic or organic acids and bases. “Pharmaceutically acceptable salts” of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Sodium salts of antisense oligonucleotides are useful and are well accepted for therapeutic administration to humans. Accordingly, in one embodiment the compounds described herein are in the form of a sodium salt.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form (i.e. a drug) within the body or cells thereof by the action of endogenous enzymes or other chemicals or conditions.

“Region” or “target region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides can be modified with any of a variety of substituents.

“Second agent” or “second therapeutic agent” means an agent that can be used in combination with a “first agent”. A second therapeutic agent can be any agent that ameliorates, inhibits or prevents metabolic and/or cardiovascular disease. A second therapeutic agent can include, but is not limited to, an siRNA or antisense oligonucleotide including antisense oligonucleotides targeting CETP or another target. A second agent can also include antibodies (e.g., anti-CETP antibodies), peptide inhibitors (e.g., CETP peptide inhibitors), cholesterol lowering agents, lipid lowering agents, glucose lowering agents and anti-inflammatory agents.

“Segments” are defined as smaller, sub-portions of regions within a nucleic acid. For example, a “target segment” means the sequence of nucleotides of a target nucleic acid to which one or more antisense compounds is targeted. “5′ target site” refers to the 5′-most nucleotide of a target segment. “3′ target site” refers to the 3′-most nucleotide of a target segment.

“Shortened” or “truncated” versions of antisense oligonucleotides or target nucleic acids taught herein have one, two or more nucleosides deleted.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum can indicate liver toxicity or liver function abnormality. For example, increased bilirubin can indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Statin” means an agent that inhibits the activity of HMG-CoA reductase.

“Subject” means a human or non-human animal selected for treatment or therapy.

“Subcutaneous administration” means administration just below the skin.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutic lifestyle change” means dietary and lifestyle changes intended to lower fat/adipose tissue mass and/or cholesterol. Such change can reduce the risk of developing heart disease, and may includes recommendations for dietary intake of total daily calories, total fat, saturated fat, polyunsaturated fat, monounsaturated fat, carbohydrate, protein, cholesterol, insoluble fiber, as well as recommendations for physical activity.

“Triglyceride” or “TO” means a lipid or neutral fat consisting of glycerol combined with three fatty acid molecules.

“Type 2 diabetes,” (also known as “type 2 diabetes mellitus” or “diabetes mellitus, type 2”, and formerly called “diabetes mellitus type 2”, “non-insulin-dependent diabetes (NIDDM)”, “obesity related diabetes”, or “adult-onset diabetes”) is a metabolic disorder that is primarily characterized by insulin resistance, relative insulin deficiency, and hyperglycemia.

“Treat” refers to administering a pharmaceutical composition to an animal to effect an alteration or improvement of a disease, disorder, or condition.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

In certain embodiments, the compounds or compositions of the invention comprise a modified oligonucleotide 10 to 30 linked nucleosides in length targeted to CETP. The CETP target can have a sequence selected from any one of SEQ ID NOs: 1-4.

In certain embodiments, the compounds or compositions of the invention comprise a modified oligonucleotide consisting of 10 to 30 nucleosides having a nucleobase sequence comprising at least 8 contiguous nucleobases complementary to an equal length portion of SEQ ID NOs: 1-4.

In certain embodiments, the compounds or compositions of the invention comprise a modified oligonucleotide consisting of 10 to 30 linked nucleosides and having a nucleobase sequence comprising at least 8, 9, 10, 11, 12, 13, 14, 15, 16, 17, 18, 19, 20, 21, 22, 23, 24, 25, 26, 27, 28, 29 or 30 contiguous nucleobases complementary to an equal length portion of SEQ ID NOs: 1-4.

In certain embodiments, the compounds or compositions of the invention can consist of 10 to 30 linked nucleosides and have a nucleobase sequence comprising at least 8, 9, 10, 11, 12, 13, 14, 15, 16, 17, 18, 19, or 20 contiguous nucleobases of any of SEQ ID NO: 41-114.

In certain embodiments, the following antisense compounds or oligonucleotides target a region of a CETP nucleic acid and effect at least a 60% inhibition of a CETP mRNA: SEQ ID NOs: 49, 53, 56, 57, 58, 59, 60, 62, 66, 70, 71, 76, 77, 78, 80, 83, 86, 89, 92, 93, 94, 96, 97, 99, 102, 105, 106, 107, 108, 109, 110, 111, 113, 114.

In certain embodiments, the following antisense compounds or oligonucleotides target a region of a CETP nucleic acid and effect at least a 65% inhibition of a CETP mRNA: SEQ ID NOs: 49, 53, 56, 57, 58, 59, 60, 62, 66, 70, 71, 76, 77, 78, 80, 83, 86, 89, 92, 93, 94, 96, 97, 99, 102, 105, 106, 108, 109, 110, 111, 113, 114.

In certain embodiments, the following antisense compounds or oligonucleotides target a region of a CETP nucleic acid and effect at least a 70% inhibition of a CETP mRNA: SEQ ID NOs: 49, 56, 57, 58, 59, 60, 62, 66, 70, 71, 77, 80, 83, 86, 93, 94, 96, 99, 102, 105, 106, 109, 110, 111, 114.

In certain embodiments, the following antisense compounds or oligonucleotides target a region of a CETP nucleic acid and effect at least a 75% inhibition of a CETP mRNA: SEQ ID NOs: 49, 56, 57, 58, 59, 60, 62, 71, 80, 86, 93, 94, 99, 102, 109, 110, 114.

In certain embodiments, the following antisense compounds or oligonucleotides target a region of a CETP nucleic acid and effect at least a 80% inhibition of a CETP mRNA: SEQ ID NOs: 57, 58, 62, 80, 86, 94, 99, 102, 109, 110, 114.

In certain embodiments, the following antisense compounds or oligonucleotides target a region of a CETP nucleic acid and effect at least a 85% inhibition of a CETP mRNA: SEQ ID NOs: 58, 62, 80, 86, 102, 109, 114.

In certain embodiments, the following antisense compounds or oligonucleotides target a region of a CETP nucleic acid and effect at least a 90% inhibition of a CETP mRNA: SEQ ID NOs: 58, 102, 109, 114.

In certain embodiments, antisense compounds or oligonucleotides target a region of a CETP nucleic acid. In certain embodiments, an antisense compound or oligonucleotide targeted to a CETP nucleic acid can target the following nucleotide regions of SEQ ID NO: 1: 132-151, 239-278, 303-435, 511-550, 614-654, 703-722, 773-812, 845-910, 996-1047, 1093-1112, 1168-1187, 1208-1297, 1318-1384, 1406-1425, 1446-1493, 1539-1727, 1763-1782.

In certain embodiment, compounds or oligonucleotides targeted to a region of a CETP nucleic acid can have a contiguous nucleobase portion that is complementary to an equal length nucleobase portion of the region. For example, the portion can be at least an 8, 9, 10, 11, 12, 13, 14, 15, 16, 17, 18, 19 or 20 contiguous nucleobases portion complementary to an equal length portion of SEQ ID NO: 1 region: 132-151, 239-278, 303-435, 511-550, 614-654, 703-722, 773-812, 845-910, 996-1047, 1093-1112, 1168-1187, 1208-1297, 1318-1384, 1406-1425, 1446-1493, 1539-1727, 1763-1782.

In certain embodiments, the following nucleotide regions of SEQ ID NO: 1, when targeted by antisense compounds or oligonucleotides, display at least 60% inhibition of CETP: 132-151, 239-258, 303-396, 416-435, 511-530, 614-654, 773-812, 845-864, 891-910, 1028-1047, 1093-1112, 1168-1187, 1238-1297, 1343-1384, 1406-1425, 1474-1493, 1539-1697, 1708-1727, 1763-1782.

In certain embodiments, the following nucleotide regions of SEQ ID NO: 1, when targeted by antisense compounds or oligonucleotides, display at least 65% inhibition of CETP: 132-151, 239-258, 303-396, 416-435, 511-530, 614-654, 773-812, 845-864, 891-910, 1028-1047, 1093-1112, 1168-1187, 1238-1297, 1343-1384, 1406-1425, 1474-1493, 1539-1590, 1613-1697, 1708-1727, 1763-1782.

In certain embodiments, the following nucleotide regions of SEQ ID NO: 1, when targeted by antisense compounds or oligonucleotides, display at least 70% inhibition of CETP: 132-151, 303-396, 416-435, 511-530, 614-654, 793-812, 891-910, 1028-1047, 1093-1112, 1258-1297, 1343-1362, 1406-1425, 1474-1493, 1539-1590, 1636-1697, 1763-1782.

In certain embodiments, the following nucleotide regions of SEQ ID NO: 1, when targeted by antisense compounds or oligonucleotides, display at least 75% inhibition of CETP: 132-151, 303-396, 416-435, 635-654, 891-910, 1093-1112, 1258-1297, 1406-1425, 1474-1493, 1636-1675, 1763-1782.

In certain embodiments, the following nucleotide regions of SEQ ID NO: 1, when targeted by antisense compounds or oligonucleotides, display at least 80% inhibition of CETP: 313-352, 416-435, 891-910, 1093-1112, 1278-1297, 1406-1425, 1474-1493, 1636-1675, 1763-1782.

In certain embodiments, the following nucleotide region of SEQ ID NO: 1, when targeted by antisense compounds or oligonucleotides, display at least 85% inhibition of CETP: 333-352, 416-435, 891-910, 1093-1112, 1474-1493, 1636-1655, 1763-1782.

In certain embodiments, the following nucleotide region of SEQ ID NO: 1, when targeted by antisense compounds or oligonucleotides, display at least 90% inhibition of CETP: 333-352, 1474-1493, 1636-1655, 1763-1782.

In certain embodiments, the compounds or compositions of the invention comprise a salt of the modified oligonucleotide.

In certain embodiments, the compounds or compositions of the invention further comprise a pharmaceutically acceptable carrier or diluent.

In certain embodiments, the nucleobase sequence of the modified oligonucleotide is at least 70%, 80%, 90%, 95% or 100% complementary to any one of SEQ ID NO: 1-4 as measured over the entirety of the modified oligonucleotide.

In certain embodiments, the compound of the invention consists of a single-stranded modified oligonucleotide.

In certain embodiments, the modified oligonucleotide consists of 8, 9, 10, 11, 12, 13, 14, 15, 16, 17, 18, 19, 20, 21, 22, 23, 24, 25, 26, 27, 28, 29 or 30 linked nucleosides. In certain embodiments, the modified oligonucleotide consists of 20 linked nucleosides.

In certain embodiments, at least one internucleoside linkage of said modified oligonucleotide is a modified internucleoside linkage. In certain embodiments, each internucleoside linkage is a phosphorothioate internucleoside linkage.

In certain embodiments, at least one nucleoside of the modified oligonucleotide comprises a modified sugar. In certain embodiments, the modified oligonucleotide comprises at least one tetrahydropyran modified nucleoside wherein a tetrahydropyran ring replaces a furanose ring. In certain embodiments each of the tetrahydropyran modified nucleoside has the structure:

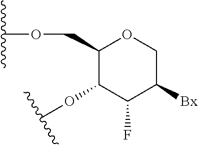

wherein Bx is an optionally protected heterocyclic base moiety. In certain embodiments, at least one modified sugar is a bicyclic sugar. In certain embodiments, at least one modified sugar comprises a 2′-O-methoxyethyl or a 4′-(CH₂)ₙ—O-2′ bridge, wherein n is 1 or 2.

In certain embodiments, at least one nucleoside of said modified oligonucleotide comprises a modified nucleobase. In certain embodiments, the modified nucleobase is a 5-methylcytosine.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, the modified oligonucleotide consists of 20 linked nucleosides, the gap segment consisting of ten linked deoxynucleosides, the 5′ wing segment consisting of five linked nucleosides, the 3′ wing segment consisting of five linked nucleosides, each nucleoside of each wing segment comprises a 2′-O-methoxyethyl sugar, each internucleoside linkage is a phosphorothioate linkage and each cytosine is a 5-methylcytosine.

In certain embodiments, the compounds or compositions of the invention comprise a modified oligonucleotide consists of 20 linked nucleosides having a nucleobase sequence comprising at least 8 contiguous nucleobases complementary to an equal length portion of any of SEQ ID NO: 1-4, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage is a phosphorothioate linkage and each cytosine residue is a 5-methylcytosine.

Certain embodiments provide methods, compounds, and compositions for inhibiting CETP expression.

Certain embodiments provide a method of reducing CETP expression in an animal comprising administering to the animal a compound of the invention described herein. In certain embodiments, the compound comprises a modified oligonucleotide 10 to 30 linked nucleosides in length targeted to CETP.

Certain embodiments provide a method of reducing CETP activity in an animal comprising administering to the animal a compound of the invention described herein. In certain embodiments, the compound comprises a modified oligonucleotide 10 to 30 linked nucleosides in length targeted to CETP.

Certain embodiments provide a method of increasing HDL levels and/or increasing HDL activity in an animal comprising administering to the animal a compound of the invention described herein. In certain embodiments, the compound comprises a modified oligonucleotide 10 to 30 linked nucleosides in length targeted to CETP. In certain embodiments, HDL level and/or HDL activity is increased by at least 5%, 10%, 20%, 30%, 35%, 40%, 45%, 50%, 55%, 60%, 65%, 70%, 75%, 80%, 85%, 90%, 95% or 100%.

Certain embodiments provide a method of reducing LDL, TG or glucose levels in an animal comprising administering to the animal a compound of the invention described herein. In certain embodiments, the compound comprises a modified oligonucleotide 10 to 30 linked nucleosides in length targeted to CETP.

Certain embodiments provide a method of reducing LDL/HDL ratio in an animal comprising administering to the animal a compound of the invention described herein. In certain embodiments, the compound comprises a modified oligonucleotide 10 to 30 linked nucleosides in length targeted to CETP.

Certain embodiments provide a method of a preventing or ameliorating metabolic or cardiovascular disease in an animal comprising administering to the animal a compound of the invention described herein. In certain embodiments, the compound comprises a modified oligonucleotide 10 to 30 linked nucleosides in length targeted to CETP. In certain embodiments, the cardiovascular disease is atherosclerosis.

Certain embodiments provide a method for treating an animal with metabolic or cardiovascular disease comprising: a) identifying said animal with metabolic or cardiovascular disease, and b) administering to said animal a therapeutically effective amount of a compound comprising a modified oligonucleotide consisting of 20 linked nucleosides and having a nucleobase sequence at least 90% complementary to any of SEQ ID NO: 1-4 as measured over the entirety of said modified oligonucleotide.

In certain embodiments, a therapeutically effective amount of the compound administered to an animal reduces metabolic or cardiovascular disease in the animal. In certain embodiments, the metabolic or cardiovascular disease is obesity, diabetes, atherosclerosis, dyslipidemia, coronary heart disease, non-alcoholic fatty liver disease (NAFLD), hyperfattyacidemia, metabolic syndrome, or a combination thereof. In certain embodiments, the cardiovascular disease is aneurysm, angina, arrhythmia, atherosclerosis, cerebrovascular disease, coronary heart disease, hypertension, dyslipidemia, hyperlipidemia or hypercholesterolemia. The dyslipidemia can be hyperlipidemia, hypercholesterolemia or hypertriglyceridemia. The NAFLD can be hepatic steatosis or steatohepatitis. The diabetes can be type 2 diabetes or type 2 diabetes with dyslipidemia.

In certain embodiments, administering the compound of the invention can result in improved insulin sensitivity, hepatic insulin sensitivity or cardiovascular outcome or a reduction in atherosclerotic plaques, atherosclerotic lesions, obesity, glucose, lipids, glucose resistance, insulin resistance, or any combination thereof.

In certain embodiments, CETP has the sequence as set forth in any of the GenBank Accession Numbers listed in Table 1 (incorporated herein as SEQ ID NOs: 1-6). In certain embodiments, CETP has the human sequence as set forth in nucleotides 10609000 to 10633000 of GenBank Accession No. NT_{—}010498.15 (incorporated herein as SEQ ID NO: 4). In certain embodiments, CETP has the human mRNA sequence as set forth in GenBank Accession No. NM_{—}000078.1 (incorporated herein as SEQ ID NO: 2).

TABLE 1
Gene Target Names and Sequences

| | | | SEQ ID |
|---|---|---|---|
| Target Name | Species | Genbank # | NO |
| CETP | Human | M30185.1 | 1 |
| CETP | Human | NM_000078.1 | 2 |
| CETP | Human | M83573.1 | 3 |
| CETP | Human | nucleotides 10609000 to 10633000 | 4 |
| | | of NT_010498.15 | |
| CETP | Rabbit | M27486.1 | 5 |
| CETP | Cynomolgus | M86343.1 | 6 |
| | monkey | | |

In certain embodiments, the animal is a human.

In certain embodiments, the compounds or compositions of the invention are designated as a first agent. In certain embodiments, the methods of the invention comprise administering a first and second agent. In certain embodiments, the first agent and the second agent are co-administered. In certain embodiments the first agent and the second agent are co-administered sequentially or concomitantly.

In certain embodiments, the second agent is a glucose-lowering agent. The glucose lowering agent can include, but is not limited to, a therapeutic lifestyle change, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, or a combination thereof. The glucose-lowering agent can include, but is not limited to metformin, sulfonylurea, rosiglitazone, meglitinide, thiazolidinedione, alpha-glucosidase inhibitor or a combination thereof. The sulfonylurea can be acetohexamide, chlorpropamide, tolbutamide, tolazamide, glimepiride, a glipizide, a glyburide, or a gliclazide. The meglitinide can be nateglinide or repaglinide. The thiazolidinedione can be pioglitazone or rosiglitazone. The alpha-glucosidase can be acarbose or miglitol.

In certain embodiments, the second agent is a lipid lowering therapy. In certain embodiments, the second agent is a LDL lowering therapy. In certain embodiments, the second agent is a TG lowering therapy. In certain embodiments, the second agent is a cholesterol lowering therapy. In certain embodiments the lipid lowering therapy can include, but is not limited to, a therapeutic lifestyle change, statins, fibrates or MTP inhibitors

In certain embodiments, administration comprises parenteral administration.

Certain embodiments provide the use of a compound as described herein for reducing CETP in an animal. In certain embodiments, the compound comprises a modified oligonucleotide 10 to 30 linked nucleosides in length targeted to CETP as shown in any of SEQ ID NO: 1-4.

Certain embodiments provide the use of a compound as described herein for increasing HDL and/or HDL activity in an animal. In certain embodiments, the compound comprises a modified oligonucleotide 10 to 30 linked nucleosides in length targeted to CETP as shown in any of SEQ ID NO: 1-4.

Certain embodiments provide the use of a compound as described herein for reducing LDL, TG or glucose levels in an animal. In certain embodiments, the compound comprises a modified oligonucleotide 10 to 30 linked nucleosides in length targeted to CETP as shown in any of SEQ ID NO: 1-4.

Certain embodiments provide the use of a compound as described herein for treating, ameliorating, delaying or preventing one or more of a metabolic disease or a cardiovascular disease, or a symptom thereof, in an animal. In certain embodiments, the compound comprises a modified oligonucleotide 10 to 30 linked nucleosides in length targeted to CETP as shown in any of SEQ ID NO: 1-4.

Certain embodiments provide the use of a compound as described herein in the manufacture of a medicament for treating, ameliorating, delaying or preventing one or more of a metabolic disease, a cardiovascular disease, or a symptom thereof. In certain embodiments, the compound comprises a modified oligonucleotide 10 to 30 linked nucleosides in length targeted to CETP as shown in any of SEQ ID NO: 1-4.

Certain embodiments provide a kit for treating, preventing, or ameliorating one or more of a metabolic disease, a cardiovascular disease, or a symptom thereof, as described herein wherein the kit comprises: a) a compound as described herein; and optionally b) an additional agent or therapy as described herein. The kit can further include instructions or a label for using the kit to treat, prevent, or ameliorate one or more of a metabolic disease or a cardiovascular disease.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound can be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to CETP nucleic acid is 10 to 30 nucleotides in length. In other words, antisense compounds are from 10 to 30 linked nucleobases. In other embodiments, the antisense compound comprises a modified oligonucleotide consisting of 8 to 80, 10-80. 12 to 50, 15 to 30, 18 to 24, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values. In some embodiments, the antisense compound is an antisense oligonucleotide.

In certain embodiments, the antisense compound comprises a shortened or truncated modified oligonucleotide. The shortened or truncated modified oligonucleotide can have a single nucleoside deleted from the 5′ end (5′ truncation), the central portion or alternatively from the 3′ end (3′ truncation). A shortened or truncated oligonucleotide can have two or more nucleosides deleted from the 5′ end, two or more nucleosides deleted from the central portion or alternatively can have two or more nucleosides deleted from the 3′ end. In certain embodiments, the deleted nucleosides can be dispersed throughout the modified oligonucleotide, for example, in an antisense compound having one or more nucleosides deleted from the 5′ end and one or more nucleosides deleted from the 3′ end.

When a single additional nucleoside is present in a lengthened oligonucletide, the additional nucleoside can be located at the 5′ or 3′ end or the central portion of the oligonucleotide. When two or more additional nucleosides are present, the added nucleosides can be adjacent to each other, for example, in an oligonucleotide having two nucleosides added to the central portion, the 5′ end (5′ addition), or alternatively to the 3′ end (3′ addition), of the oligonucleotide. Alternatively, the added nucleoside can be dispersed throughout the antisense compound, for example, in an oligonucleotide having one or more nucleoside added to the 5′ end, one or more nucleoside added to the 3′ end and/or one or more nucleoside added to the central portion.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a CETP nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound can optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2′-modified nucleosides (such 2′-modified nucleosides can include 2′-MOE, and 2′-O—CH₃, among others), and bicyclic sugar modified nucleosides (such bicyclic sugar modified nucleosides can include those having a 4′-(CH2)n-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6, 5-8-5, 1-8-1, 2-6-2, 2-13-2, 1-8-2, 2-8-3, 3-10-2, 1-18-2 or 2-18-2.

In certain embodiments, the antisense compound as a “wingmer” motif, having a wing-gap or gap-wing configuration, i.e. an X-Y or Y-Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13 or 5-13.

In certain embodiments, antisense compounds targeted to a CETP nucleic acid possess a 5-10-5 gapmer motif.

In certain embodiments, an antisense compound targeted to a CETP nucleic acid has a gap-widened motif.

### Target Nucleic Acids, Target Regions and Nucleotide Sequences

Nucleotide sequences that encode CETP include, without limitation, the following: the sequence as set forth in GenBank Accession No. M30185.1 (incorporated herein as SEQ ID NO: 1), GenBank Accession No. NM_{—}000078.1 (incorporated herein as SEQ ID NO: 2), GenBank Accession No. M83573.1 (incorporated herein as SEQ ID NO: 3) or in nucleotides 10609000 to 10633000 of GenBank Accession No. NT_{—}010498.15 (incorporated herein as SEQ ID NO: 4). It is understood that the sequence set forth in each SEQ ID NO in the Examples contained herein is independent of any modification to a sugar moiety, an internucleoside linkage, or a nucleobase. As such, antisense compounds defined by a SEQ ID NO can comprise, independently, one or more modifications to a sugar moiety, an internucleoside linkage, or a nucleobase. Antisense compounds described by Isis Number (Isis No) indicate a combination of nucleobase sequence and motif

In certain embodiments, a target region is a structurally defined region of the target nucleic acid. For example, a target region can encompass a 3′ UTR, a 5′ UTR, an exon, an intron, an exon/intron junction, a coding region, a translation initiation region, translation termination region, or other defined nucleic acid region. The structurally defined regions for CETP can be obtained by accession number from sequence databases such as NCBI and such information is incorporated herein by reference. In certain embodiments, a target region can encompass the sequence from a 5′ target site of one target segment within the target region to a 3′ target site of another target segment within the target region.

In certain embodiments, a “target segment” is a smaller, sub-portion of a target region within a nucleic acid. For example, a target segment can be the sequence of nucleotides of a target nucleic acid to which one or more antisense compounds are targeted. “5′ target site” refers to the 5′-most nucleotide of a target segment. “3′ target site” refers to the 3′-most nucleotide of a target segment.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments can be found within a 5′ UTR, a coding region, a 3′ UTR, an intron, an exon, or an exon/intron junction. Target segments containing a start codon or a stop codon are also suitable target segments. A suitable target segment can specifically exclude a certain structurally defined region such as the start codon or stop codon.

The determination of suitable target segments can include a comparison of the sequence of a target nucleic acid to other sequences throughout the genome. For example, the BLAST algorithm can be used to identify regions of similarity amongst different nucleic acids. This comparison can prevent the selection of antisense compound sequences that can hybridize in a non-specific manner to sequences other than a selected target nucleic acid (i.e., non-target or off-target sequences).

There can be variation in activity (e.g., as defined by percent reduction of target nucleic acid levels) of the antisense compounds within an active target region. In certain embodiments, reductions in CETP mRNA levels are indicative of inhibition of CETP protein expression. Reductions in levels of a CETP protein are also indicative of inhibition of target mRNA expression. Further, phenotypic changes, such as a reduction of the level of proinflammatory cytokines or glucose, can be indicative of inhibition of CETP mRNA and/or protein expression.

### Hybridization

In some embodiments, hybridization occurs between an antisense compound disclosed herein and a CETP nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrooke and Russell, Molecular Cloning: A Laboratory Manual, 3^{rd} Ed., 2001). In certain embodiments, the antisense compounds provided herein are specifically hybridizable with a CETP nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a CETP nucleic acid).

An antisense compound can hybridize over one or more segments of a CETP nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 75%, 80%, 85%, 86%, 87%, 88%, 89%, 90%, 91%, 92%, 93%, 94%, 95%, 96%, 97%, 98%, 99%, or 100% complementary to a CETP nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining non-complementary nucleobases can be clustered or interspersed with complementary nucleobases and need not be contiguous to each other or to complementary nucleobases. As such, an antisense compound which is 18 nucleobases in length having 4 (four) non-complementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound can be fully complementary to a CETP nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully (100%) complementary to a target sequence that is 400 nucleobases long, so long as there is a corresponding 20 nucleobase portion of the target nucleic acid that is fully complementary to the antisense compound. Fully complementary can also be used in reference to a specified portion of the first and for the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is “fully complementary” to the target sequence if the target sequence has a corresponding 20 nucleobase portion wherein each nucleobase is complementary to the 20 nucleobase portion of the antisense compound. At the same time, the entire 30 nucleobase antisense compound can be fully complementary to the target sequence, depending on whether the remaining 10 nucleobases of the antisense compound are also complementary to the target sequence.

The location of a non-complementary nucleobase can be at the 5′ end or 3′ end of the antisense compound. Alternatively, the non-complementary nucleobase or nucleobases can be at an internal position of the antisense compound. When two or more non-complementary nucleobases are present, they can be either contiguous (i.e. linked) or non-contiguous. In one embodiment, a non-complementary nucleobase is located in the wing segment of a gapmer antisense oligonucleotide.

In certain embodiments, antisense compounds that are, or are up to 10, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a CETP nucleic acid, or specified portion thereof.

In certain embodiments, antisense compounds that are, or are up to 10, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a CETP nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 10 nucleobase portion of a target segment. In certain embodiments, the antisense compounds are complementary to at least a 15 nucleobase portion of a target segment. Also contemplated are antisense compounds that are complementary to at least an 8, 9, 10, 11, 12, 13, 14, 15, 16, 17, 18, 19, 20, or more nucleobase portion of a target segment, or a range defined by any two of these values.

### Identity

The antisense compounds provided herein can also have a defined percent identity to a particular nucleotide sequence, SEQ ID NO, or sequence of a compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases can be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a CETP nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise a chemically modified ribofuranose ring moiety. Examples of chemically modified ribofuranose rings include, without limitation, addition of substitutent groups (including 5′ and 2′ substituent groups; bridging of non-geminal ring atoms to form bicyclic nucleic acids (BNA); replacement of the ribosyl ring oxygen atom with S, N(R), or C(R1)(R)2 (R═H, C₁-C₁₂ alkyl or a protecting group); and combinations thereof. Examples of chemically modified sugars include, 2′-F-5′-methyl substituted nucleoside (see, PCT International Application WO 2008/101157, published on Aug. 21, 2008 for other disclosed 5′,2′-bis substituted nucleosides), replacement of the ribosyl ring oxygen atom with S with further substitution at the 2′-position (see, published U.S. Patent Application US2005/0130923, published on Jun. 16, 2005), or, alternatively, 5′-substitution of a BNA (see, PCT International Application WO 2007/134181, published on Nov. 22, 2007, wherein LNA is substituted with, for example, a 5′-methyl or a 5′-vinyl group).

Examples of nucleosides having modified sugar moieties include, without limitation, nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)2OCH₃ substituent groups. The substituent at the 2′ position can also be selected from allyl, amino, azido, thio, O-allyl, O—C1-C10 alkyl, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C1-C10 alkyl.

As used herein, “bicyclic nucleosides” refer to modified nucleosides comprising a bicyclic sugar moiety. Examples of bicyclic nucleosides include, without limitation, nucleosides comprising a bridge between the 4′ and the 2′ ribosyl ring atoms. In certain embodiments, antisense compounds provided herein include one or more bicyclic nucleosides wherein the bridge comprises a 4′ to 2′ bicyclic nucleoside. Examples of such 4′ to 2′ bicyclic nucleosides, include, but are not limited to, one of the formulae: 4′-(CH₂)—O-2′ (LNA); 4′-(CH₂)—S-2; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845, issued on Jul. 15, 2008); 4′-C(CH₃)(CH₃)—O-2′ and analogs thereof, (see, published International Application WO2009/006478, published Jan. 8, 2009); 4′-CH₂—N(OCH₃)-2′ and analogs thereof (see, published PCT International Application WO2008/150729, published Dec. 11, 2008); 4′-CH₂—O—N(CH₃)-2′ (see published U.S. Patent Application US2004/0171570, published Sep. 2, 2004); 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S. A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; International applications WO 2004/106356, WO 1994/14226, WO 2005/021570, and WO 2007/134181; U.S. Patent Publication Nos. US2004/0171570 (U.S. Pat. No. 7,696,345), US2007/0287831 (U.S. Pat. No. 7,547,684), and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591 (WO 2008/150729), PCT/US2008/066154 (WO 2008/154401), and PCT/US2008/068922 (WO 2009/006478). Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfonyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or, —C(RₐR_{b})—O—N(R)—. In certain embodiments, the bridge is 4′-CH₂-2′,4′-(CH₂)₂-2′,4′-(CH₂)₃-2′,4′-CH₂—O-2′,4′-(CH₂)₂—O-2′,4′-CH₂—O—N(R)-2′, and 4′-CH₂—N(R)—O-2′-, wherein each R is, independently, H, a protecting group, or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA, (G) methylene-thio (4′-CH₂—S-2′) BNA, (H) methylene-amino (4′-CH2-N(R)-2′) BNA, (I) methyl carbocyclic (4′-CH₂—CH(CH₃)-2′) BNA, and (J) propylene carbocyclic (4′-(CH₂)₃-2′) BNA as depicted below.

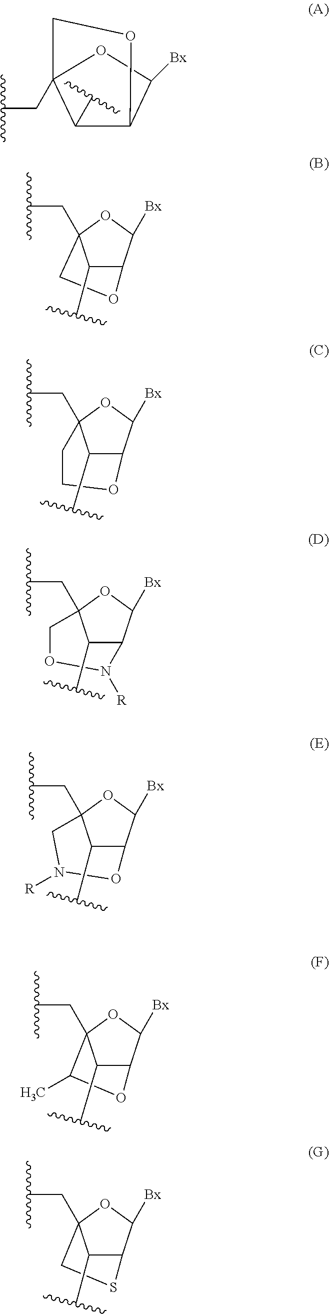

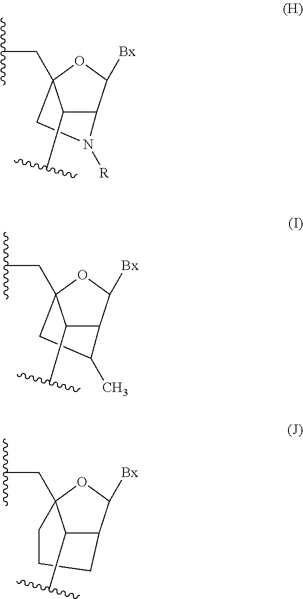

wherein Bx is the base moiety and R is, independently, H, a protecting group, or C₁-C₁₂ alkyl.

In certain embodiments, bicyclic nucleoside having Formula I:

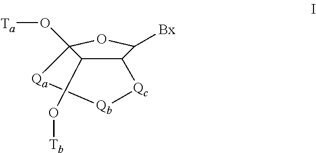

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O—, or —N(R_{c})—O—CH₂;

R_{c} is C₁-C₁₂ alkyl or an amino protecting group; and

Tₐ and T_{b} are each, independently, H, a hydroxyl protecting group, a conjugate group, a reactive phosphorus group, a phosphorus moiety, or a covalent attachment to a support medium.

In certain embodiments, bicyclic nucleoside having Formula II:

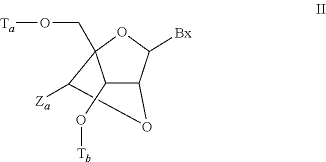

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol, or substituted thio.

In one embodiment, each of the substituted groups is, independently, mono or poly substituted with substituent groups independently selected from halogen, oxo, hydroxyl, OJ_{c}, N_{c}J_{d}, SJ_{c}, N₃, OC(═X)J_{c} and NJₑC(═X)NJ_{c}J_{d}, wherein each J_{c}, J_{d}, and Jₑ is, independently, H, C₁-C₆ alkyl, or substituted C₁-C₆ alkyl and X is O or NJ_{c}.

In certain embodiments, bicyclic nucleoside having Formula III:

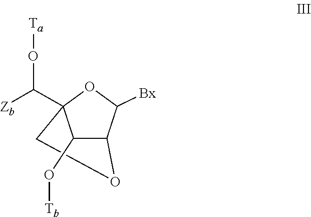

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, or substituted acyl (C(═O)—).

In certain embodiments, bicyclic nucleoside having Formula IV:

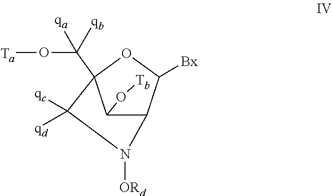

wherein:

Bx is a heterocyclic base moiety;

Tₐ and T_{b} are each, independently H, a hydroxyl protecting group, a conjugate group, a reactive phosphorus group, a phosphorus moiety, or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl, C₁-C₆ alkoxyl, substituted C₁-C₆ alkoxyl, acyl, substituted acyl, C₁-C₆ aminoalkyl, or substituted C₁-C₆ aminoalkyl;

In certain embodiments, bicyclic nucleoside having Formula V:

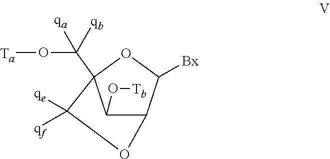

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl, or substituted C₁-C₁₂ alkyl.

The synthesis and preparation of the methyleneoxy (4′-CH₂—O-2′) BNA monomers adenine, cytosine, guanine, 5-methyl-cytosine, thymine, and uracil, along with their oligomerization, and nucleic acid recognition properties have been described (see, e.g., Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA, and 2′-thio-BNAs, have also been prepared (see, e.g., Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (see, e.g., Wengel et al., WO 99/14226). Furthermore, synthesis of 2′-amino-BNA, a novel conformationally restricted high-affinity oligonucleotide analog, has been described in the art (see, e.g., Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleoside having Formula VI:

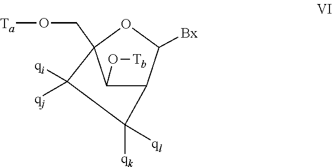

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

each q₁, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, SJⱼ, SOJⱼ, SO₂Jⱼ, NJⱼJₖ, N₃, CN, C(═O)OJⱼ, C(═O)NJⱼJₖ, O—C(═O)NJⱼJₖ, N(H)C(═NH)NJⱼJₖ, N(H)C(═O)NJⱼJₖ, or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl, or substituted C₁-C₁₂ alkyl.

One carbocyclic bicyclic nucleoside having a 4′-(CH₂)₃-2′ bridge and the alkenyl analog, bridge 4′-CH═CH—CH₂-2′, have been described (see, e.g., Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge between the 4′ and the 2′ position of the furanose ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl; substituted alkyl; alkenyl; alkynyl; alkaryl; aralkyl; O-alkaryl or O-aralkyl; SH; SCH₃; OCN; Cl; Br; CN; CF₃; OCF₃; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; and a group for improving pharmacokinetic properties, a group for improving the pharmacodynamic properties of an antisense compound, and other substituents having similar properties. In certain embodiments, modified nucleosides comprise a 2′-MOE side chain (see, e.g., Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (see, e.g., Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, C J. Bioorg. & Med. Chem. (2002) 10:841-854), fluoro HNA (F—HNA), or those compounds having Formula X:

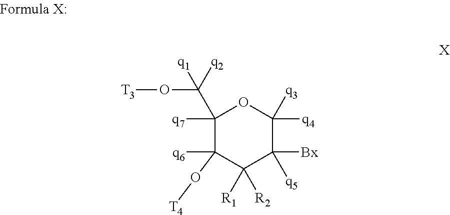

wherein independently for each of said at least one tetrahydropyran nucleoside analog of Formula X:

Bx is a heterocyclic base moiety;

T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula X are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula X are provided wherein one of R₁ and R₂ is F. In certain embodiments, R₁ is fluoro and R₂ is H, R₁ is methoxy and R₂ is H, and R₁ is methoxyethoxy and R₂ is H.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring and nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example, at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Such ring systems can undergo various additional substitutions to enhance activity.

Methods for the preparations of modified sugars are well known to those skilled in the art.

In nucleotides having modified sugar moieties, the nucleobase moieties (natural, modified, or a combination thereof) are maintained for hybridization with an appropriate nucleic acid target.

In certain embodiments, antisense compounds comprise one or more nucleotides having modified sugar moieties. In certain embodiments, the modified sugar moiety is 2′-MOE. In certain embodiments, the 2′-MOE modified nucleotides are arranged in a gapmer motif. In certain embodiments, the modified sugar moiety is a bicyclic nucleoside. In certain embodiments, the bicyclic nucleoside comprises a (4′-CH(CH₃)-0-2′) bridge. In certain embodiments, the (4′-CH(CH₃)—O-2′) bicyclic nucleotides are arranged throughout the wings of a gapmer motif. In certain embodiments, the bicyclic nucleotide is a cEt. In certain embodiments, the cEt bicyclic nucleotides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a CETP nucleic acid comprise one or more modified nucleobases. In certain embodiments, gap-widened antisense oligonucleotides targeted to a CETP nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides can be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a CETP nucleic acid can be utilized in pharmaceutical compositions by combining the antisense compound with a suitable pharmaceutically acceptable diluent or carrier.

In certain embodiments, the “pharmaceutical carrier” or “excipient” is a pharmaceutically acceptable solvent, suspending agent or any other pharmacologically inert vehicle for delivering one or more nucleic acids to an animal. The excipient can be liquid or solid and can be selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients, which do not deleteriously react with nucleic acids, suitable for parenteral or non-parenteral administration can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a CETP nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or an oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of CETP nucleic acids can be tested in vitro in a variety of cell types. Cell types used for such analyses are available from commercial vendors (e.g. American Type Culture Collection, Manassus, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, Huh7 (hepatocellular carcinoma) cells, primary hepatocytes, A549 cells, GM04281 fibroblasts and LLC-MK2 cells.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluence in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE 2000® (Invitrogen, Carlsbad, Calif.). Antisense oligonucleotide is mixed with LIPOFECTAMINE 2000® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Another reagent used to introduce antisense oligonucleotides into cultured cells includes Cytofectin® (Invitrogen, Carlsbad, Calif.). Antisense oligonucleotide is mixed with Cytofectin® in OPTI-MEM® 1 reduced serum medium (Invitrogen, Carlsbad, Calif.) to achieve the desired concentration of antisense oligonucleotide and a Cytofectin® concentration that typically ranges 2 to 12 ug/mL per 100 nM antisense oligonucleotide.

Another reagent used to introduce antisense oligonucleotides into cultured cells includes Oligofectamine™ (Invitrogen Life Technologies, Carlsbad, Calif.). Antisense oligonucleotide is mixed with Oligofectamine™ in Opti-MEM™-1 reduced serum medium (Invitrogen Life Technologies, Carlsbad, Calif.) to achieve the desired concentration of oligonucleotide with an Oligofectamine™ to oligonucleotide ratio of approximately 0.2 to 0.8 μL per 100 nM.

Another reagent used to introduce antisense oligonucleotides into cultured cells includes FuGENE 6 (Roche Diagnostics Corp., Indianapolis, Ind.). Antisense oligomeric compound was mixed with FuGENE 6 in 1 mL of serum-free RPMI to achieve the desired concentration of oligonucleotide with a FuGENE 6 to oligomeric compound ratio of 1 to 4 μL of FuGENE 6 per 100 nM.

Another technique used to introduce antisense oligonucleotides into cultured cells includes electroporation (Sambrooke and Russell, Molecular Cloning: A Laboratory Manual, 3^{rd} Ed., 2001).

Cells are treated with antisense oligonucleotides by routine methods. Cells are typically harvested 16-24 hours after antisense oligonucleotide treatment, at which time RNA or protein levels of target nucleic acids are measured by methods known in the art and described herein (Sambrooke and Russell in Molecular Cloning. A Laboratory Manual. Third Edition. Cold Spring Harbor laboratory Press, Cold Spring Harbor, N.Y. 2001). In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art (Sambrooke and Russell in Molecular Cloning. A Laboratory Manual. Third Edition. Cold Spring Harbor laboratory Press, Cold Spring Harbor, N.Y. 2001). Antisense oligonucleotides are typically used at concentrations ranging from 1 nM to 300 nM when transfected with LIPOFECTAMINE2000® (Invitrogen, Carlsbad, Calif.), Lipofectin® (Invitrogen, Carlsbad, Calif.) or Cytofectin™ (Genlantis, San Diego, Calif.). Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of a CETP nucleic acid can be assayed in a variety of ways known in the art (Sambrooke and Russell in Molecular Cloning. A Laboratory Manual. Third Edition. Cold Spring Harbor laboratory Press, Cold Spring Harbor, N.Y. 2001). For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels can be accomplished by quantitative real-time PCR using the ABI PRISM® 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT and real-time-PCR reactions are carried out by methods well known to those skilled in the art.

Gene (or RNA) target quantities obtained by real time PCR can be normalized using either the expression level of a gene whose expression is constant, such as cyclophilin A, or by quantifying total RNA using RIBOGREEN® (Invitrogen, Inc. Carlsbad, Calif.). Cyclophilin A expression is quantified by real time PCR, by being run simultaneously with the target, multiplexing, or separately. Total RNA is quantified using RIBOGREEN® RNA quantification reagent (Invitrogen, Inc. Carlsbad, Calif.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a CETP nucleic acid. Methods for designing real-time PCR probes and primers are well known in the art, and can include the use of software such as PRIMER EXPRESS® Software (Applied Biosystems, Foster City, Calif.).

Gene target quantities obtained by RT, real-time PCR were normalized using either the expression level of GAPDH or Cyclophilin A, genes whose expression are constant, or by quantifying total RNA using RiboGreen™ (Molecular Probes, Inc. Eugene, Oreg.). GAPDH or Cyclophilin A expression can be quantified by RT, real-time PCR, by being run simultaneously with the target, multiplexing, or separately. Total RNA was quantified using RiboGreen™ RNA quantification reagent (Molecular Probes, Inc. Eugene, Oreg.).

Presented in Table 2 are primers and probes used to measure GAPDH or Cyclophilin A expression in the cell types described herein. The PCR probes have JOE or FAM covalently linked to the 5′ end and TAMRA or MGB covalently linked to the 3′ end, where JOE or FAM is the fluorescent reporter dye and TAMRA or MGB is the quencher dye. In some cell types, primers and probe designed to a sequence from a different species are used to measure expression. For example, a human GAPDH primer and probe set can be used to measure GAPDH expression in monkey-derived cells and cell lines.

TABLE 2
GAPDH primers and probes for use in real-time PCR

| | | | | SEQ |
|---|---|---|---|---|
| Target Name | Species | Sequence Description | Sequence (5′ to 3′) | ID NO |
| GAPDH | Human | Forward Primer | CAACGGATTTGGTCGTATTGG | 7 |
| GAPDH | Human | Reverse Primer | GGCAACAATATCCACTTTACCAGAGT | 8 |
| GAPDH | Human | Probe | CGCCTGGTCACCAGGGCTGCT | 9 |
| GAPDH | Human | Forward Primer | GAAGGTGAAGGTCGGAGTC | 10 |
| GAPDH | Human | Reverse Primer | GAAGATGGTGATGGGATTTC | 11 |
| GAPDH | Human | Probe | CAAGCTTCCCGTTCTCAGCC | 12 |
| GAPDH | Human | Probe | TGGAATCATATTGGAACATG | 13 |
| GAPDH | Mouse | Forward Primer | GGCAAATTCAACGGCACAGT | 14 |
| GAPDH | Mouse | Reverse Primer | GGGTCTCGCTCCTGGAAGAT | 15 |
| GAPDH | Mouse | Probe | AAGGCCGAGAATGGGAAGCTTGTCATC | 16 |
| GAPDH | Rat | Forward Primer | TGTTCTAGAGACAGCCGCATCTT | 17 |
| GAPDH | Rat | Reverse Primer | CACCGACCTTCACCATCTTGT | 18 |
| GAPDH | Rat | Probe | TTGTGCAGTGCCAGCCTCGTCTCA | 19 |
| Cyclophilin A | Human | Forward Primer | TGCTGGACCCAACACAAATG | 20 |
| Cyclophilin A | Human | Reverse Primer | TGCCATCCAACCACTCAGTC | 21 |
| Cyclophilin A | Human | Probe | TTCCCAGTTTTTCATCTGCACTGCCA | 22 |
| Cyclophilin A | Human | Forward Primer | GACGGCGAGCCCTTGG | 23 |
| Cyclophilin A | Human | Reverse Primer | TGCTGTCTTTGGGACCTTGTC | 24 |
| Cyclophilin A | Human | Probe | CCGCGTCTCCTTTGAGCTGTTTGC | 25 |
| Cyclophilin A | Human | Forward Primer | GCCATGGAGCGCTTTGG | 26 |
| Cyclophilin A | Human | Reverse Primer | TCCACAGTCAGCAATGGTGATC | 27 |
| Cyclophilin A | Human | Probe | TCCAGGAATGGCAAGACCAGCAAGA | 28 |
| Cyclophilin A | Mouse | Forward Primer | TCGCCGCTTGCTGCA | 29 |
| Cyclophilin A | Mouse | Reverse Primer | ATCGGCCGTGATGTCGA | 30 |
| Cyclophilin A | Mouse | Probe | CCATGGTCAACCCCACCGTGTTC | 31 |
| Cyclophilin A | Rat | Forward Primer | CCCACCGTGTTCTTCGACA | 32 |
| Cyclophilin A | Rat | Reverse Primer | AAACAGCTCGAAGCAGACGC | 33 |
| Cyclophilin A | Rat | Probe | CACGGCTGATGGCGAGCCC | 34 |

Probes and primers for use in real-time PCR are designed to hybridize to target-specific sequences. The target-specific PCR probes can have FAM covalently linked to the 5′ end and TAMRA or MGB covalently linked to the 3′ end, where FAM is the fluorescent dye and TAMRA or MGB is the quencher dye.

### Analysis of Protein Levels

Antisense inhibition of CETP nucleic acids can be assessed by measuring CETP protein levels. Protein levels of CETP can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of CETP and produce phenotypic changes. Testing can be performed in normal animals, or in experimental disease models. For administration to animals, antisense oligonucleotides are formulated in a pharmaceutically acceptable diluent, such as phosphate-buffered saline. Administration includes parenteral routes of administration. Calculation of antisense oligonucleotide dosage and dosing frequency depends upon factors such as route of administration and animal body weight. Following a period of treatment with antisense oligonucleotides, RNA is isolated from tissue and changes in CETP nucleic acid expression are measured. Changes in CETP protein levels are also measured.

### Certain Indications

In certain embodiments, provided herein are methods of treating an individual comprising administering one or more pharmaceutical compositions as described herein. In certain embodiments, the individual has inflammatory, metabolic or cardiovascular disease.

Accordingly, provided herein are methods for ameliorating a symptom associated with inflammatory, metabolic or cardiovascular disease in a subject in need thereof. In certain embodiments, provided is a method for reducing the rate of onset of a symptom associated with inflammatory, metabolic or cardiovascular disease. In certain embodiments, provided is a method for reducing the severity of a symptom associated with inflammatory, metabolic or cardiovascular disease. In such embodiments, the methods comprise administering to an individual in need thereof a therapeutically effective amount of a compound targeted to a CETP nucleic acid.

In certain embodiments, administration of a therapeutically effective amount of an antisense compound targeted to a CETP nucleic acid is accompanied by monitoring of CETP levels or markers of inflammatory, metabolic or cardiovascular or other disease process associated with the expression of CETP, to determine an individual's response to administration of the antisense compound. An individual's response to administration of the antisense compound is used by a physician to determine the amount and duration of therapeutic intervention.

In certain embodiments, administration of an antisense compound targeted to a CETP nucleic acid results in reduction of CETP expression by at least about 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to CETP are used for the preparation of a medicament for treating a patient suffering or susceptible to inflammatory, metabolic or cardiovascular disease.

In certain embodiments, the methods described herein include administering a compound comprising a modified oligonucleotide having an 8, 9, 10, 11, 12, 13, 14, 15, 16, 17, 18, 19 or 20 contiguous nucleobase portion.

### Administration

The compounds or pharmaceutical compositions of the present invention can be administered in a number of ways depending upon whether local or systemic treatment is desired and upon the area to be treated. Administration can be topical (including ophthalmic and to mucous membranes including vaginal and rectal delivery), intradermal (for local treatment of skin fibrosis or scarring), pulmonary, (e.g., by local inhalation or insufflation of powders or aerosols, including by nebulizer; intratracheal, intranasal, epidermal and transdermal), oral or parenteral.

In certain embodiments, the compounds and compositions as described herein are administered parenterally. Parenteral administration includes intravenous, intra-arterial, subcutaneous, intraperitoneal or intramuscular injection or infusion; or intracranial, e.g., intrathecal or intraventricular, administration.

In certain embodiments, parenteral administration is by infusion. Infusion can be chronic or continuous or short or intermittent. In certain embodiments, infused pharmaceutical agents are delivered with a pump.

In certain embodiments, parenteral administration is by injection. The injection can be delivered with a syringe or a pump. In certain embodiments, the injection is a bolus injection. In certain embodiments, the injection is administered directly to a tissue or organ.

In certain embodiments, formulations for parenteral, intrathecal or intraventricular administration can include sterile aqueous solutions which can also contain buffers, diluents and other suitable additives such as, but not limited to, penetration enhancers, carrier compounds and other pharmaceutically acceptable carriers or excipients.

In certain embodiments, formulations for topical administration of the compounds or compositions of the invention can include, but is not limited to, pharmaceutical carriers, excipients, sterile and non-sterile aqueous solutions, non-aqueous solutions in common solvents such as alcohols, or solutions of the compounds or compositions in liquid or solid oil bases. The solutions can also contain buffers, diluents and other suitable additives. Formulations for topical administration can include transdermal patches, ointments, lotions, creams, gels, drops, suppositories, sprays, liquids and powders.

In certain embodiments, formulations for oral administration of the compounds or compositions of the invention can include, but is not limited to, pharmaceutical carriers, excipients, powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In certain embodiments, oral formulations are those in which compounds of the invention are administered in conjunction with one or more penetration enhancers, surfactants and chelators.

### Dosing

In certain embodiments, pharmaceutical compositions are administered according to a dosing regimen (e.g., dose, dose frequency, and duration) wherein the dosing regimen can be selected to achieve a desired effect. The desired effect can be, for example, reduction of CETP or the prevention, reduction, amelioration or slowing the progression of a disease or condition associated with CETP.

In certain embodiments, the variables of the dosing regimen are adjusted to result in a desired concentration of pharmaceutical composition in a subject. “Concentration of pharmaceutical composition” as used with regard to dose regimen can refer to the compound, oligonucleotide, or active ingredient of the pharmaceutical composition. For example, in certain embodiments, dose and dose frequency are adjusted to provide a tissue concentration or plasma concentration of a pharmaceutical composition at an amount sufficient to achieve a desired effect.

Dosing is dependent on severity and responsiveness of the disease state to be treated, with the course of treatment lasting from several days to several months, or until a cure is effected or a diminution of the disease state is achieved. Dosing is also dependent on drug potency and metabolism. In certain embodiments, dosage is from 0.01 μg to 100 mg per kg of body weight, or within a range of 0.001 mg to 600 mg dosing, and may be given once or more daily, weekly, monthly or yearly, or even once every 2 to 20 years. Following successful treatment, it may be desirable to have the patient undergo maintenance therapy to prevent the recurrence of the disease state, wherein the oligonucleotide is administered in maintenance doses, ranging from 0.01 μg to 100 mg per kg of body weight, once or more daily, to once every 20 years or ranging from 0.001 mg to 600 mg dosing.

### Certain Combination Therapies

In certain embodiments, a first agent comprising the modified oligonucleotide of the invention is co-administered with one or more secondary agents. In certain embodiments, such second agents are designed to treat the same inflammatory, metabolic or cardiovascular disease as the first agent described herein. In certain embodiments, such second agents are designed to treat a different disease, disorder, or condition as the first agent described herein. In certain embodiments, such second agents are designed to treat an undesired side effect of one or more pharmaceutical compositions as described herein. In certain embodiments, such first agent are designed to treat an undesired side effect of a second agent. In certain embodiments, second agents are co-administered with the first agent to treat an undesired effect of the first agent. In certain embodiments, second agents are co-administered with the first agent to produce a combinational effect. In certain embodiments, second agents are co-administered with the first agent to produce a synergistic effect. In certain embodiments, the co-administration of the first and second agents permits use of lower dosages than would be required to achieve a therapeutic or prophylactic effect if the agents were administered as independent therapy.

In certain embodiments, a first agent and one or more second agents are administered at the same time. In certain embodiments, the first agent and one or more second agents are administered at different times. In certain embodiments, the first agent and one or more second agents are prepared together in a single pharmaceutical formulation. In certain embodiments, the first agent and one or more second agents are prepared separately.

In certain embodiments, second agents include, but are not limited to, a glucose-lowering agent, a cholesterol or lipid lowering therapy or an anti-inflammatory or inflammation lowering agent. The glucose lowering agent can include, but is not limited to, a therapeutic lifestyle change, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, or a combination thereof. The glucose-lowering agent can include, but is not limited to metformin, sulfonylurea, rosiglitazone, meglitinide, thiazolidinedione, alpha-glucosidase inhibitor or a combination thereof. The sulfonylurea can be acetohexamide, chlorpropamide, tolbutamide, tolazamide, glimepiride, a glipizide, a glyburide, or a gliclazide. The meglitinide can be nateglinide or repaglinide. The thiazolidinedione can be pioglitazone or rosiglitazone. The alpha-glucosidase can be acarbose or miglitol. The cholesterol or lipid lowering therapy can include, but is not limited to, a therapeutic lifestyle change, statins, bile acids sequestrants, nicotinic acid and fibrates. The statins can be atorvastatin, fluvastatin, lovastatin, pravastatin, rosuvastatin and simvastatin and the like. The bile acid sequestrants can be colesevelam, cholestyramine, colestipol and the like. The fibrates can be gemfibrozil, fenofibrate, clofibrate and the like. The inflammation lowering agent can include, but is not limited to, a therapeutic lifestyle change, a steroid or a NSAID. The steroid can be a corticosteroid. The NSAID can be an aspirin, acetaminophen, ibuprofen, naproxen, COX inhibitors, indomethacin and the like.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of Human CETP mRNA in SW872 Liposarcoma Cells

Antisense oligonucleotides targeted to a CETP nucleic acid were tested for their effect on CETP mRNA transcript in vitro. Cultured SW872 liposarcoma cells at a density of 50,000 cells per well in a 24-well plate were transfected using lipofectin reagent with 150 nM antisense oligonucleotide. After approximately 24 hours, RNA was isolated from the cells and CETP mRNA levels were measured by quantitative real-time PCR. A human primer probe set (forward sequence GGCATCCCTGAGGTCATGTC, designated herein as SEQ ID NO: 38; reverse sequence GGCTCACGCCTTTGCTGTT, designated herein as SEQ ID NO: 39; probe sequence CGGCTCGAGGTAGTGTTTACAGCCCTC, designated herein as SEQ ID NO: 40) was used to quantitate CETP mRNA. CETP mRNA transcript levels were adjusted according to total RNA content, as measured by the house-keeping gene, GAPDH mRNA levels. GAPDH was measured using a primer probe set with the sequence as set forth in SEQ ID NOs: 10, 11, 13. Results are presented as percent inhibition of CETP, relative to untreated control cells.

The antisense oligonucleotides in Table 3 are 5-10-5 gapmers, where the gap segment comprises ten 2′-deoxynucleosides and each wing segment comprises five 2′-MOE nucleosides. Each nucleotide in the 5′ wing segment and each nucleotide in the 3′ wing segment has a 2′-MOE modification. The internucleoside linkages throughout each gapmer are phosphorothioate (P═S) linkages. All cytosine residues throughout each gapmer are 5-methylcytosines. ‘Target start site’ indicates the 5′-most nucleotide to which the antisense oligonucleotide is targeted. ‘Target stop site’ indicates the 3′-most nucleotide to which the antisense oligonucleotide is targeted. All the antisense oligonucleotides listed in Table 3 target human CETP mRNA, designated herein as SEQ ID NO: 1 (GENBANK Accession No. M30185.1).

TABLE 3
Inhibition of human CETP mRNA in SW872 cells by 5-10-5
gapmers targeting SEQ ID NO: 1

| Target | Target | | | % | SEQ |
|---|---|---|---|---|---|
| Start Site | Stop Site | ISIS No. | Sequence (5′ to 3′) | inhibition | ID NO |
| 1 | 20 | 349444 | TCCTGGCCCCAGAGATTCAC | 20 | 41 |
| 12 | 31 | 349445 | CAGCAGGGTCTTCCTGGCCC | 6 | 42 |
| 33 | 52 | 349446 | GGAACATGAGGCTCTTCCGG | 0 | 43 |
| 57 | 76 | 349447 | TATGTATGTCCGCCCAGCCC | 15 | 44 |
| 62 | 81 | 349448 | CCGTATATGTATGTCCGCCC | 0 | 45 |
| 82 | 101 | 349449 | CGAGCCGTTCAGCCTGGAGC | 28 | 46 |
| 102 | 121 | 349450 | GCAGTGGTGTGTAAGTGGCC | 21 | 47 |
| 122 | 141 | 349451 | GCAGCCAGCATGGTTATCAG | 23 | 48 |
| 132 | 151 | 349452 | CAGGACTGTGGCAGCCAGCA | 75 | 49 |
| 155 | th | 349453 | GCATTGCCCAGCAGGGCCAG | 45 | 50 |
| 176 | 195 | 349454 | GTGCCTTTGGAGCAGGCATG | 46 | 51 |
| 196 | 215 | 349455 | CGATGCCTGCCTCGTGCGAG | 35 | 52 |
| 239 | 258 | 349456 | TCGTGGTTCAACACCAGGAG | 68 | 53 |
| 259 | 278 | 349457 | TCTGGATCACCTTGGCAGTC | 52 | 54 |
| 283 | 302 | 349458 | GGTAGCTGGCTCGCTGGAAG | 18 | 55 |
| 303 | 322 | 349459 | CTTCTCGCCCGTGATATCTG | 79 | 56 |
| 313 | 332 | 349460 | GCATCATGGCCTTCTCGCCC | 80 | 57 |
| 333 | 352 | 349461 | ATACTTGACTTGGCCAAGGA | 97 | 58 |
| 353 | 372 | 349462 | ATCTGGATGTTGTGCAACCC | 76 | 59 |
| 377 | 396 | 349463 | CTGCTGGCGATGGACAAGTG | 77 | 60 |
| 397 | 416 | 349464 | CTTCCACCAGCTCCACCTGG | 50 | 61 |
| 416 | 435 | 349465 | GAGACATCAATGGACTTGGC | 87 | 62 |
| 436 | 455 | 349466 | CCACAGACACGTTCTGAATG | 15 | 63 |
| 470 | 489 | 349467 | GTGGTGTAGCCATACTTCAG | 35 | 64 |
| 491 | 510 | 349468 | TCAATACCCAGCCACCAGGC | 33 | 65 |
| 511 | 530 | 349469 | TCTCGAAGTCAATGGACTGA | 70 | 66 |
| 531 | 550 | 349470 | GAGGTCAATGGCAGAGTCGA | 59 | 67 |
| 551 | 570 | 349471 | GTCAGCTGTGTGTTGATCTG | 32 | 68 |
| 571 | 590 | 349472 | GCACTCTACCAGAGTCACAG | 39 | 69 |
| 614 | 633 | 349473 | AGCAGCTTATGGAAAGACAG | 72 | 70 |
| 635 | 654 | 349474 | CGCTCCCCTTGGAGATGCAG | 76 | 71 |
| 655 | 674 | 349475 | GCTTGATCCACCCAGGCTCT | 5 | 72 |
| 703 | 722 | 349476 | CCTTCAGGACCAGCTTCAGG | 59 | 73 |
| 723 | 742 | 349477 | GATCTCTTTGCAGATCTGTC | 41 | 74 |
| 743 | 762 | 349478 | ATGATGTTAGAGATGACGTT | 48 | 75 |
| 773 | 792 | 349479 | CTGGCAGCCCTTGTCTGGAC | 67 | 76 |
| 793 | 812 | 349480 | TGTCTCCATCTGAAAGGATG | 74 | 77 |
| 845 | 864 | 349481 | AGGTAGGAGGCTGTGATGAC | 66 | 78 |
| 871 | 890 | 349482 | TGAAATGACCCTTGTGATGG | 51 | 79 |
| 891 | 910 | 349483 | CTCTGAGACATTCTTGTAGA | 85 | 80 |
| 955 | 974 | 349484 | ACCAGAAGTACAGCATGCGG | 38 | 81 |
| 996 | 1015 | 349486 | GAAAGCTACCTTGGCCAGCG | 55 | 82 |
| 1028 | 1047 | 349487 | CCCATCAGGCTGAGCATGAG | 73 | 83 |
| 1048 | 1067 | 349488 | GCACTGCCTTGAACTCGTCT | 47 | 84 |
| 1068 | 1087 | 349489 | GTTGAAGCCCCAGGTCTCCA | 48 | 85 |
| 1093 | 1112 | 349490 | CCTCTTGGAAGATTTCCTGG | 89 | 86 |
| 1105 | 1124 | 349491 | AGCCGCCGACAACCTCTTGG | 45 | 87 |
| 1148 | 1167 | 349492 | GGCATCTTGAGGCAGTGGAC | 39 | 88 |
| 1168 | 1187 | 349493 | TGTTTTGGCAGGAGATCTTG | 67 | 89 |
| 1188 | 1207 | 349494 | AGAATTGACCACGACTCCCT | 41 | 90 |
| 1208 | 1227 | 349495 | AGGAATTTCACCATCACTGA | 58 | 91 |
| 1238 | 1257 | 349496 | ACAGAATGTTGCTGGTCTGG | 69 | 92 |
| 1258 | 1277 | 349497 | CCTCTTCAAATGTGTAAGCT | 78 | 93 |
| 1278 | 1297 | 349498 | CTGGACGGTAGTCACGATAT | 80 | 94 |
| 1318 | 1337 | 349499 | CCAAGAGGCTTAAGAAGAGC | 55 | 95 |
| 1343 | 1362 | 349500 | ACAGTCTTTGGTGTAATCTG | 72 | 96 |
| 1365 | 1384 | 349501 | GCTGCTCTCAGTCAAGTTGG | 67 | 97 |
| 1386 | 1405 | 349502 | GAAGCTCTGGATGGACTCGG | 43 | 98 |
| 1406 | 1425 | 349503 | GCGGTGATCATTGACTGCAG | 81 | 99 |
| 1426 | 1445 | 349504 | TGACCTCAGGGATGCCCACA | 45 | 100 |
| 1446 | 1465 | 349505 | CACTACCTCGAGCCGAGACA | 55 | 101 |
| 1474 | 1493 | 349506 | CGCCTTTGCTGTTCATGAGG | 92 | 102 |
| 1495 | 1514 | 349507 | TGATGATGTCGAAGAGGCTC | 19 | 103 |
| 1517 | 1536 | 349508 | TCTCGAGTGATAATCTCAGG | 24 | 104 |
| 1539 | 1558 | 349509 | CATCTGCAGCAGCAGGAAGC | 70 | 105 |
| 1571 | 1590 | 133837 | TCCACCAGCAGGTGCTCAGG | 70 | 106 |
| 1594 | 1613 | 349510 | TCTAGCTCAAGCTCTGGAGG | 64 | 107 |
| 1613 | 1632 | 349511 | CCCGACCTCCTTGGAGACTT | 66 | 108 |
| 1636 | 1655 | 349512 | TTGCCTTCTGCTACAAGCCC | 92 | 109 |
| 1656 | 1675 | 349513 | TCCAGCTGTGAGCCTGGTGC | 83 | 110 |
| 1678 | 1697 | 349514 | ACGCTGGAGGAGACACCAGG | 71 | 111 |
| 1688 | 1707 | 349515 | AACTTCCACCACGCTGGAGG | 53 | 112 |
| 1708 | 1727 | 349516 | CATCTCCGTACTCCTAACCC | 65 | 113 |
| 1763 | 1782 | 349517 | CAGCACTTTAATGCCAGTGG | 100 | 114 |

### Example 2

### Dose-Dependent Antisense Inhibition of Human CETP in SW872 Liposarcoma Cells

Several of the antisense oligonucleotides exhibiting in vitro inhibition of CETP in SW872 liposarcoma cells (see Example 1) were tested at various doses. Cells were plated at a density of 50,000 cells per well in a 24-well plate and transfected using Lipofectin reagent with 50 nM, 150 nM, and 300 nM concentrations of each antisense oligonucleotide. After approximately 16 hours, RNA was isolated from the cells and CETP mRNA levels were measured by quantitative real-time PCR. CETP mRNA levels were normalized to total RNA content, as measured by the house-keeping gene, GAPDH mRNA levels. Results are presented in Table 4 as percent inhibition of CETP, relative to untreated control cells.

TABLE 4
Dose-dependent antisense inhibition of human
CETP in SW872 liposarcoma cells

| ISIS No | 50 nM | 150 nM | 300 nM |
|---|---|---|---|
| 349461 | 75 | 80 | 88 |
| 349465 | 96 | 87 | 96 |
| 349483 | 100 | 98 | 85 |
| 349490 | 97 | 93 | 88 |
| 349506 | 93 | 86 | 93 |
| 349512 | 86 | 98 | 97 |
| 349513 | 97 | 100 | 95 |
| 349517 | 100 | 98 | 98 |

### Example 3

### Dose-Dependent Antisense Inhibition of Human CETP in SW872 Liposarcoma Cells

ISIS 349513 and ISIS 349517, which exhibited significant dose-dependent inhibition of CETP in SW872 liposarcoma cells (see Example 2) were further tested at various doses. ISIS 17291 (GACAAGTGGCTGATCTGGAT, 6-8-6 MOE (SEQ ID NO: 115)), ISIS 17302 (GCTTGCCTTCTGCTACAAGC, 6-8-6 MOE (SEQ ID NO: 116)), and ISIS 17305 (CCAGTGGGCCTTTAGGATAG, 6-8-6 MOE (SEQ ID NO: 117), first disclosed in U.S. Ser. No. 11/031,827 (incorporated by reference herein), were also tested under the same conditions. Cells were plated at a density of 50,000 cells per well in a 24-well plate and transfected using Lipofectin reagent with 50 nM, 150 nM, and 300 nM concentrations of each antisense oligonucleotide. After approximately 16 hours, RNA was isolated from the cells and CETP mRNA levels were measured by quantitative real-time PCR. CETP mRNA levels were normalized to total RNA content, as measured by the house-keeping gene, GAPDH mRNA levels. Results are presented in Table 5 as percent inhibition of CETP, relative to untreated control cells.

TABLE 5
Dose-dependent antisense inhibition of human
CETP in SW872 liposarcoma cells

| ISIS No | 50 nM | 150 nM | 300 nM |
|---|---|---|---|
| 17291 | 35 | 0 | 0 |
| 17302 | 0 | 41 | 45 |
| 17305 | 18 | 44 | 55 |
| 349513 | 87 | 89 | 91 |
| 349517 | 76 | 91 | 93 |

### Example 4

### Antisense Inhibition of Human Cholesteryl Ester Transfer Protein (CETP) mRNA in Primary Hepatocytes from Human CETP Transgenic Mice

Transgenic mice expressing human CETP, controlled by its natural flanking region, increase expression of this gene in response to hypercholesterolemia (Jiang, X. C. et al., J. Clin. Invest. 1992. 90: 1290-1295). These mice were utilized for the following studies.

Antisense oligonucleotides targeted to a CETP nucleic acid and described in Example 1 were tested for their effect on CETP mRNA transcript in vitro. Cultured primary hepatocytes from huCETP transgenic mice, at a density of 17,500 cells per well in a 96-well plate, were transfected using cytofectin reagent with 100 nM or 200 nM antisense oligonucleotide. After approximately 24 hours, RNA was isolated from the cells and CETP mRNA levels were measured by quantitative real-time PCR. CETP mRNA transcript levels were adjusted according to total RNA content, as measured by the house-keeping gene, GAPDH mRNA levels. Results are presented in Table 6 as percent inhibition of CETP, relative to untreated control cells.

TABLE 6
Percent inhibition of human CETP mRNA in primary hepatocytes
of huCETP transgenic mice by 5-10-5 gapmers

| ISIS No. | 200 nM | 100 nM |
|---|---|---|
| 349444 | 0 | 59 |
| 349445 | 71 | 74 |
| 349446 | 36 | 34 |
| 349447 | 28 | 57 |
| 349448 | 40 | 53 |
| 349449 | 63 | 68 |
| 349450 | 59 | 89 |
| 349451 | 54 | 42 |
| 349452 | 51 | 60 |
| 349453 | 26 | 2 |
| 349454 | 68 | 69 |
| 349455 | 17 | 25 |
| 349456 | 51 | 74 |
| 349457 | 30 | 42 |
| 349458 | 32 | 65 |
| 349459 | 63 | 50 |
| 349460 | 76 | 67 |
| 349461 | 52 | 65 |
| 349462 | 76 | 60 |
| 349463 | 62 | 58 |
| 349464 | 56 | 8 |
| 349465 | 66 | 50 |
| 349466 | 81 | 57 |
| 349467 | 51 | 57 |
| 349468 | 50 | 53 |
| 349469 | 66 | 40 |
| 349470 | 49 | 64 |
| 349471 | 66 | 70 |
| 349472 | 45 | 25 |
| 349473 | 61 | 55 |
| 349474 | 48 | 64 |
| 349475 | 0 | 0 |
| 349476 | 60 | 49 |
| 349477 | 47 | 44 |
| 349478 | 33 | 6 |
| 349479 | 66 | 80 |
| 349480 | 40 | 46 |
| 349481 | 65 | 79 |
| 349482 | 65 | 11 |
| 349483 | 63 | 77 |
| 349484 | 52 | 34 |
| 349485 | 36 | 14 |
| 349486 | 53 | 72 |
| 349487 | 68 | 84 |
| 349488 | 87 | 77 |
| 349489 | 67 | 47 |
| 349490 | 80 | 36 |
| 349491 | 74 | 60 |
| 349492 | 58 | 59 |
| 349493 | 62 | 51 |
| 349494 | 57 | 85 |
| 349495 | 74 | 61 |
| 349496 | 87 | 57 |
| 349497 | 79 | 71 |
| 349498 | 84 | 76 |
| 349499 | 80 | 80 |
| 349500 | 77 | 41 |
| 349501 | 45 | 54 |
| 349502 | 61 | 49 |
| 349503 | 89 | 83 |
| 349504 | 92 | 78 |
| 349505 | 99 | 98 |
| 349506 | 99 | 95 |
| 349507 | 73 | 71 |
| 349508 | 86 | 76 |
| 349509 | 83 | 22 |
| 349510 | 88 | 65 |
| 349511 | 95 | 74 |
| 349512 | 96 | 86 |
| 349513 | 92 | 73 |
| 349514 | 92 | 84 |
| 349515 | 94 | 94 |
| 349516 | 86 | 78 |
| 349517 | 98 | 85 |

### Example 5

### Antisense Inhibition of Human CETP in huCETP Transgenic Mice Fed a Normal Chow Diet

ISIS 349511 (SEQ ID NO: 108), ISIS 349512 (SEQ ID NO: 109), ISIS 349515 (SEQ ID NO: 112), and ISIS 349517 (SEQ ID NO: 114), which demonstrated statistically significant dose-dependent inhibition in vitro, were evaluated for their ability to reduce human CETP mRNA transcript in vivo.

### Treatment

Male huCETP transgenic mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

The mice were divided into five treatment groups. The first four groups received intraperitoneal injections of ISIS 349511, ISIS 349512, ISIS 349515, or ISIS 349517 at a dose of 50 mg/kg twice per week for 6 weeks. The fifth group received intraperitoneal injections of saline twice weekly for 6 weeks. The saline-injected group served as the control group to which the oligonucleotide-treated group was compared.

Inhibition of CETP mRNA

Twenty four hours after the final dose, the animals were sacrificed and liver tissue was isolated. Liver RNA was isolated for real-time PCR analysis of CETP. Two primer probe sets, hCETP (forward sequence CAGCTGACCTGTGACTCTGGTAGA, designated herein as SEQ ID NO: 118; reverse sequence CAGCTTATGGAAAGACAGGTAGCA, designated herein as SEQ ID NO: 119; probe sequence TGCGGACCGATGCCCCTGAX, designated herein as SEQ ID NO: 120) and hCETP2_LTS00182 (forward sequence GGCATCCCTGAGGTCATGTC, designated herein as SEQ ID NO: 38; reverse sequence GGCTCACGCCTTTGCTGTT, designated herein as SEQ ID NO: 39: probe sequence CGGCTCGAGGTAGTGTTTACAGCCCTCX, designated herein as SEQ ID NO: 40), were used. These primer probe sets target the CETP mRNA transcript at different regions. As presented in Table 7, treatment with antisense oligonucleotides reduced CETP mRNA expression. The primer probe sets were used individually and collectively to measure CETP mRNA expression and gave similar results in all three cases. The RNA expression levels were normalized to murine Cyclophilin, a house-keeping gene, which is constitutively expressed. Cyclophilin levels were measured using the primer probe set described in Table 2. The results are expressed as percent inhibition of CETP mRNA, relative to the PBS control.

TABLE 7
Percent inhibition of liver CETP mRNA expression in transgenic mice

| | | | Combined |
|---|---|---|---|
| | Primer probe set | Primer probe set | primer probe |
| ISIS No. | hCETP | hCETP2_LTS00182 | sets |
| 349511 | 90 | 86 | 88 |
| 349512 | 96 | 94 | 95 |
| 349515 | 63 | 52 | 57 |
| 349517 | 99 | 95 | 97 |

### Inhibition of CETP Protein

Plasma levels of CETP protein were measured by ELISA (Alpco, N.H.). The results are presented in Table 8 and demonstrate significant inhibition of protein levels of CETP by all the ISIS antisense oligonucleotides. The results are expressed as percentage inhibition compared to the PBS control.

TABLE 8
Percent inhibition of plasma CETP protein levels in transgenic mice

| ISIS No. | % inhibition |
|---|---|
| 349511 | 75 |
| 349512 | 76 |
| 349515 | 40 |
| 349517 | 86 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.) (Nyblom, H. et al., Alcohol & Alcoholism 39: 336-339, 2004; Tietz N W (Ed): Clinical Guide to Laboratory Tests, 3rd ed. W. B. Saunders, Philadelphia, Pa., 1995). Plasma concentrations of ALT (alanine transaminase) at weeks 3 and 6 were measured and the results are presented in Table 9 expressed in IU/L. Most of the ISIS oligonucleotides were considered tolerable in the mice, as demonstrated by their liver transaminase profile.

TABLE 9
Alanine transaminase levels (IU/L) of transgenic mice at weeks 3 and 6

| | Week 3 | Week 6 |
|---|---|---|
| PBS | 32 | 51 |
| ISIS 349511 | 32 | 42 |
| ISIS 349512 | 60 | 651 |
| ISIS 349515 | 66 | 82 |
| ISIS 349517 | 35 | 69 |

### Body and Organ Weights

The body weights of the mice were measured pre-dose and regularly during the treatment period. The body weights are presented in Table 10, and are expressed in grams. Liver, spleen and kidney weights were measured at the end of the study, and are presented in Table 11.

TABLE 10
Body weights of transgenic mice

| | Week 0 | Week 1 | Week 2 | Week 4 | Week 6 |
|---|---|---|---|---|---|
| PBS | 25 | 25 | 25 | 26 | 26 |
| ISIS 349511 | 19 | 21 | 23 | 25 | 26 |
| ISIS 349512 | 19 | 21 | 24 | 26 | 28 |
| ISIS 349515 | 25 | 26 | 27 | 28 | 28 |
| ISIS 349517 | 25 | 27 | 28 | 28 | 29 |

TABLE 11
Organ weights of transgenic mice at week 6

| | Liver (g) | Kidney (mg) | Spleen (mg) |
|---|---|---|---|
| PBS | 1.3 | 315.6 | 79.4 |
| ISIS 349511 | 1.3 | 312.5 | 76.0 |
| ISIS 349512 | 2.1 | 333.8 | 86.8 |
| ISIS 349515 | 1.7 | 349.5 | 89.3 |
| ISIS 349517 | 1.7 | 355.5 | 89.0 |

### Glucose Levels

Plasma glucose values were determined using a Beckman Glucose Analyzer II (Beckman Coulter) by a glucose oxidase method (Lott, J. A. et al., Clin. Chem. 21: 1754-1760, 1975). The results are presented in Table 12 expressed in mg/dL.

TABLE 12
Glucose levels in transgenic mice

| | Week 3 | Week 6 |
|---|---|---|
| PBS | 306 | 228 |
| ISIS 349511 | 286 | 245 |
| ISIS 349512 | 267 | 248 |
| ISIS 349515 | 275 | 237 |
| ISIS 349517 | 333 | 254 |

### Cholesterol and Triglyceride Levels

Plasma cholesterol were extracted at weeks 3 and 6 by the method of Bligh and Dyer (Bligh, E. G. and Dyer, W. J. Can. J. Biochem. Physiol. 37: 911-917, 1959) and measured with an Olympus clinical analyzer (Hitachi Olympus AU400e, Melville, N.Y.). HDL, LDL and VLDL cholesterol were individually measured by HPLC. Triglyceride levels were measured with the use of a commercially available triglyceride kit (DCL Triglyceride Reagent; Diagnostic Chemicals Ltd.). The results are presented in Tables 13 and 14 and are expressed in mg/dL. There was a significant increase in HDL levels in mice treated with ISIS oligonucleotides. The percentage of HDL levels in relation to total cholesterol levels is presented in Table 15.

TABLE 13
Cholesterol and triglyceride levels (mg/dL) in transgenic
mice at week 3

| | Total | | | |
|---|---|---|---|---|
| | cholesterol | HDL | LDL | Triglycerides |
| PBS | 70 | 45 | 20 | 88 |
| ISIS 349511 | 83 | 60 | 15 | 87 |
| ISIS 349512 | 85 | 62 | 14 | 100 |
| ISIS 349515 | 89 | 60 | 19 | 83 |
| ISIS 349517 | 79 | 59 | 14 | 68 |

TABLE 14
Cholesterol and triglyceride levels (mg/dL) in transgenic
mice at week 6

| | Total | | | | |
|---|---|---|---|---|---|
| | cholesterol | HDL | LDL | VLDL | Triglycerides |
| PBS | 75 | 48 | 19 | 8 | 120 |
| ISIS 349511 | 88 | 66 | 17 | 4 | 84 |
| ISIS 349512 | 134 | 100 | 31 | 3 | 92 |
| ISIS 349515 | 87 | 66 | 18 | 4 | 115 |
| ISIS 349517 | 75 | 55 | 15 | 4 | 103 |

TABLE 15
Percentage of HDL in total cholesterol in transgenic mice

| | Week 3 | Week 6 |
|---|---|---|
| PBS | 65 | 65 |
| ISIS 349511 | 73 | 76 |
| ISIS 349512 | 74 | 74 |
| ISIS 349515 | 68 | 75 |
| ISIS 349517 | 75 | 74 |

### Example 6

### Antisense Inhibition of Human CETP in huCETP Transgenic Mice Fed a Normal Chow or Cholesterol Enriched Diet

ISIS 349511 (SEQ ID NO: 108) and ISIS 349517 (SEQ ID NO: 114) were further evaluated for their ability to reduce human CETP mRNA transcript in vivo.

### Treatment

Male huCETP transgenic mice were maintained on a 12-hour light/dark cycle. One group of mice was fed ad libitum normal Purina mouse chow and a second group was fed 0.5% cholesterol-enriched chow (Harlan Teklad TD.97234, Harlan Laboratories, Indianapolis, Ind.). Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

The normal chow-fed mice were divided into three treatment groups. The cholesterol-enriched chow-fed mice were divided into three treatment groups. Two chow-fed groups received subcutaneous injections of ISIS 349511 or ISIS 349517 at a dose of 50 mg/kg twice per week for 6 weeks. Similarly, the cholesterol-enriched groups received subcutaneous injections of ISIS 349511 or ISIS 349517 at a dose of 50 mg/kg twice per week for 6 weeks. One chow-fed group and one cholesterol-enriched chow-fed group received subcutaneous injections of saline twice weekly for 6 weeks. The saline-injected groups served as the control groups to which the corresponding oligonucleotide-treated groups were compared.

Inhibition of CETP mRNA

Twenty four hours after the final dose, the animals were sacrificed and liver tissue was isolated. Liver RNA was isolated for real-time PCR analysis of CETP. The primer probe set hCETP was used to measure CETP levels. As presented in Table 16, treatment with antisense oligonucleotides reduced CETP mRNA expression. The RNA expression levels were normalized to murine Cyclophilin. The results are expressed as percent inhibition of CETP mRNA, relative to the PBS control. There was no difference in inhibition of CETP mRNA levels in normal chow-fed and cholesterol-enriched chow fed mice.

TABLE 16
Percent inhibition of liver CETP mRNA expression in transgenic mice

| ISIS No. | Diet | % inhibition |
|---|---|---|
| 349511 | Normal chow | 89 |
| | Cholesterol chow | 88 |
| 349517 | Normal chow | 95 |
| | Cholesterol chow | 98 |

### Inhibition of CETP Protein

Plasma levels of CETP protein were measured by ELISA (Alpco, N.H.). The results are presented in Table 17 and demonstrate significant inhibition of protein levels of CETP by all the ISIS antisense oligonucleotides. The results are expressed as percentage inhibition compared to the PBS control.

TABLE 17
Percent inhibition of plasma CETP protein levels in transgenic mice

| ISIS No. | Diet | Week 3 | Week 6 |
|---|---|---|---|
| 349511 | Normal chow | 81 | 85 |
| | Cholesterol chow | 89 | 85 |
| 349517 | Normal chow | 95 | 91 |
| | Cholesterol chow | 97 | 96 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.) (Nyblom, H. et al., Alcohol & Alcoholism 39: 336-339, 2004; Tietz N W (Ed): Clinical Guide to Laboratory Tests, 3rd ed. W. B. Saunders, Philadelphia, Pa., 1995). Plasma concentrations of ALT (alanine transaminase) and AST (aspartate transaminase) at weeks 0, 3 and 6 were measured and the results are presented in Tables 18 and 19 expressed in IU/L.

TABLE 18
Alanine transaminase levels (IU/L) of transgenic mice

| | Diet | Week 0 | Week 3 | Week 6 |
|---|---|---|---|---|
| PBS | Normal chow | 21 | 33 | 25 |
| | Cholesterol chow | 45 | 32 | 22 |
| ISIS | Normal chow | 16 | 22 | 22 |
| 349511 | Cholesterol chow | 28 | 28 | 66 |
| ISIS | Normal chow | 17 | 24 | 158 |
| 349517 | Cholesterol chow | 19 | 56 | 76 |

TABLE 19
Aspartate transaminase levels (IU/L) of transgenic mice

| | Diet | Week 0 | Week 3 | Week 6 |
|---|---|---|---|---|
| PBS | Normal chow | 68 | 77 | 93 |
| | Cholesterol chow | 120 | 48 | 44 |
| ISIS | Normal chow | 89 | 47 | 81 |
| 349511 | Cholesterol chow | 49 | 44 | 123 |
| ISIS | Normal chow | 75 | 61 | 196 |
| 349517 | Cholesterol chow | 54 | 72 | 113 |

### Organ Weights

Liver, spleen and kidney weights were measured at the end of the study, and are presented in Table 20.

TABLE 20
Body weights of transgenic mice

| | | Liver | Kidney | Spleen |
|---|---|---|---|---|
| | Diet | (g) | (mg) | (mg) |
| PBS | Normal chow | 0.9 | 263 | 78 |
| | Cholesterol chow | 1.4 | 363 | 92 |
| ISIS | Normal chow | 1.0 | 248 | 74 |
| 349511 | Cholesterol chow | 1.4 | 305 | 80 |
| ISIS | Normal chow | 1.6 | 279 | 113 |
| 349517 | Cholesterol chow | 1.8 | 317 | 93 |

### Glucose Levels

Plasma glucose values were determined using a Beckman Glucose Analyzer II (Beckman Coulter) by a glucose oxidase method (Lott, J. A. et al., Clin. Chem. 21: 1754-1760, 1975). The results are presented in Table 21 expressed in mg/dL.

TABLE 21
Glucose levels in transgenic mice

| | Diet | Week 0 | Week 3 | Week 6 |
|---|---|---|---|---|
| PBS | Normal chow | 228 | 258 | 230 |
| | Cholesterol chow | 213 | 328 | 298 |
| ISIS | Normal chow | 239 | 288 | 206 |
| 349511 | Cholesterol chow | 238 | 289 | 225 |
| ISIS | Normal chow | 263 | 257 | 211 |
| 349517 | Cholesterol chow | 277 | 245 | 240 |

### Cholesterol and Triglyceride Levels

Plasma cholesterol were extracted at weeks 3 and 6 by the method of Bligh and Dyer (Bligh, E. G. and Dyer, W. J. Can. J. Biochem. Physiol. 37: 911-917, 1959) and measured on weeks 0, 3 and 6 with an Olympus clinical analyzer (Hitachi Olympus AU400e, Melville, N.Y.). HDL, LDL and VLDL cholesterol were individually measured by HPLC. Triglyceride levels were measured on weeks 0, 3 and 6 with the use of a commercially available triglyceride kit (DCL Triglyceride Reagent; Diagnostic Chemicals Ltd.). VLDL levels were measured on week 6. The results are presented in Tables 22-26 and are expressed in mg/dL.

TABLE 22
Total cholesterol levels (mg/dL) in transgenic mice

| | Diet | Week 0 | Week 3 | Week 6 |
|---|---|---|---|---|
| PBS | Normal chow | 223 | 217 | 273 |
| | Cholesterol chow | 216 | 770 | 856 |
| ISIS | Normal chow | 254 | 227 | 327 |
| 349511 | Cholesterol chow | 252 | 714 | 809 |
| ISIS | Normal chow | 237 | 238 | 375 |
| 349517 | Cholesterol chow | 201 | 739 | 695 |

TABLE 23
LDL cholesterol levels (mg/dL) in transgenic mice

| | Diet | Week 0 | Week 3 | Week 6 |
|---|---|---|---|---|
| PBS | Normal chow | 149 | 151 | 167 |
| | Cholesterol chow | 141 | 646 | 618 |
| ISIS | Normal chow | 176 | 148 | 178 |
| 349511 | Cholesterol chow | 179 | 576 | 558 |
| ISIS | Normal chow | 162 | 146 | 220 |
| 349517 | Cholesterol chow | 134 | 597 | 486 |

TABLE 24
HDL cholesterol levels (mg/dL) in transgenic mice

| | Diet | Week 0 | Week 3 | Week 6 |
|---|---|---|---|---|
| PBS | Normal chow | 52 | 46 | 74 |
| | Cholesterol chow | 49 | 71 | 127 |
| ISIS | Normal chow | 51 | 56 | 98 |
| 349511 | Cholesterol chow | 51 | 85 | 136 |
| ISIS | Normal chow | 53 | 64 | 100 |
| 349517 | Cholesterol chow | 49 | 78 | 122 |

TABLE 25
VLDL cholesterol levels (mg/dL) in transgenic mice

| | Diet | Week 6 |
|---|---|---|
| PBS | Normal chow | 32 |
| | Cholesterol chow | 111 |
| ISIS 349511 | Normal chow | 50 |
| | Cholesterol chow | 115 |
| ISIS 349517 | Normal chow | 55 |
| | Cholesterol chow | 88 |

TABLE 26
Triglyceride levels (mg/dL) in transgenic mice

| | Diet | Week 0 | Week 3 | Week 6 |
|---|---|---|---|---|
| PBS | Normal chow | 74 | 85 | 56 |
| | Cholesterol chow | 98 | 76 | 92 |
| ISIS | Normal chow | 80 | 90 | 101 |
| 349511 | Cholesterol chow | 101 | 94 | 99 |
| ISIS | Normal chow | 66 | 97 | 91 |
| 349517 | Cholesterol chow | 84 | 107 | 102 |

### Example 7

### Effect of Antisense Inhibition of CETP in Mouse Model for Cardiovascular Disease and Comparison with Treatment with Nicotinic Acid in Elevating HDL Cholesterol Levels

A transgenic mouse model of cardiovascular disease has been developed at ISIS Pharmaceuticals and is further described in international application PCT/US10/20435 (incorporated by reference herein). The mouse model comprises a hemizygous copy of human cholesteryl ester transfer protein (huCETP^{+/-}), a hemizygous copy of human apolipoprotein B (huApoB^{+/-}) and has a partial deficiency of murine low-density lipoprotein receptor (mLDLr^{+/-}). The mouse model is useful for screening prophylactic and/or therapeutic agents for hypercholesterolemia and/or cardiovascular diseases such as atherosclerosis and heart disease. In this study, huApoB^{+/-}huCETP^{+/-}mLDLr^{+/-} mice were also utilized as a control.

The efficacy of ISIS 349511, ISIS 349517 and 1% nicotinic acid to raise HDL cholesterol levels was evaluated in this model.

### Treatment

Male transgenic mice were maintained on a 12-hour light/dark cycle and were fed ad libitum normal Purina mouse chow. Animals were acclimated for at least 7 days in the research facility before initiation of the experiment. Antisense oligonucleotides (ASOs) were prepared in buffered saline (PBS) and sterilized by filtering through a 0.2 micron filter. Oligonucleotides were dissolved in 0.9% PBS for injection.

The huApoB^{+/-}huCETP^{+/-}mLDLr^{+/-} mice were divided into five groups of 5-7 mice each. Two such groups received subcutaneous injections of ISIS 349511 or ISIS 349517 at a dose of 25 mg/kg twice per week for 6 weeks. One group of mice received subcutaneous injections of 2 g of nicotinic acid per kg per day for 6 weeks, and also was fed a diet containing 1% nicotinic acid. One group of mice received subcutaneous injections of ISIS 141923 (CCTTCCCTGAAGGTTCCTCC, 5-10-5 MOE gapmer, SEQ ID NO: 121), a control oligonucleotide which has no known murine target. One group of mice received subcutaneous injections of ISIS 349511 PBS twice per week for 6 weeks. The saline-injected group served as the control group to which oligonucleotide-treated groups were compared. One group of five huApoB^{+/-}huCETP^{+/-}mLDLr^{+/-} mice received subcutaneous injections of ISIS 349517 at a dose of 25 mg/kg twice weekly for 6 weeks. This group served a negative control for the effects of the antisense oligonucleotides against CETP. The groups are further described in Table 27, and are designated letters A-F, by which they will be referred from henceforth.

TABLE 27
Treatment groups

| Group | Treatment | | |
|---|---|---|---|
| ID | type | Diet | Animal type |
| A | PBS | Chow | huApoB^{+/-}huCETP^{+/-}mLDLr^{+/-} |
| B | ISIS 141923 | Chow | huApoB^{+/-}huCETP^{+/-}mLDLr^{+/-} |
| C | ISIS 349511 | Chow | huApoB^{+/-}huCETP^{+/-}mLDLr^{+/-} |
| D | ISIS 349517 | Chow | huApoB^{+/-}huCETP^{+/-}mLDLr^{+/-} |
| E | Nicotinic | Chow | huApoB^{+/-}huCETP^{+/-}mLDLr^{+/-} |
| | acid | +1% | |
| | | nicotinic | |
| | | acid | |
| F | ISIS 349517 | chow | huApoB^{+/-}huCETP^{-/-}mLDLr^{+/-} |

Inhibition of CETP mRNA

Twenty four hours after the final dose, the animals were sacrificed and liver tissue was isolated. Liver RNA was isolated for real-time PCR analysis of CETP. The primer probe set hCETP was used to measure CETP levels. As presented in Table 28, treatment with antisense oligonucleotides (Groups C and D) reduced CETP mRNA expression. The RNA expression levels were normalized to murine Cyclophilin. The results are expressed as percent inhibition of CETP mRNA, relative to the PBS control (Group A). The transgenic mice treated with nicotinic acid (Group E) showed no inhibition data, as expected. The control oligonucleotide, ISIS 141923, also showed no inhibition potential for CETP mRNA, as expected (Group B). The negative control (Group F) was not tested for CETP reduction.

TABLE 28
Percent inhibition of liver CETP mRNA expression in transgenic mice

| Group ID | % inhibition |
|---|---|
| B | 0 |
| C | 70 |
| D | 86 |
| E | 0 |

### Cholesterol and Triglyceride Levels

Plasma cholesterol were extracted at weeks 0, 3 and 6 by the method of Bligh and Dyer (Bligh, E. G. and Dyer, W. J. Can. J. Biochem. Physiol. 37: 911-917, 1959) and measured with an Olympus clinical analyzer (Hitachi Olympus AU400e, Melville, N.Y.). HDL and LDL cholesterol were individually measured by HPLC. Triglyceride levels were measured on weeks 0, 3 and 6 with the use of a commercially available triglyceride kit (DCL Triglyceride Reagent; Diagnostic Chemicals Ltd.). The results are presented in Tables 29-33 and are expressed in mg/dL.

TABLE 29
Total cholesterol levels (mg/dL) in transgenic mice

| Group ID | Week 0 | Week 3 | Week 6 |
|---|---|---|---|
| A | 201 | 220 | 223 |
| B | 201 | 228 | 235 |
| C | 208 | 212 | 223 |
| D | 193 | 171 | 197 |
| E | 195 | 169 | 170 |
| F | 158 | 175 | 177 |

TABLE 30
LDL cholesterol levels (mg/dL) in transgenic mice

| Group ID | Week 0 | Week 3 | Week 6 |
|---|---|---|---|
| A | 126 | 146 | 142 |
| B | 123 | 149 | 150 |
| C | 129 | 126 | 132 |
| D | 116 | 87 | 109 |
| E | 122 | 102 | 102 |
| F | 73 | 91 | 95 |

TABLE 31
HDL cholesterol levels (mg/dL) in transgenic mice

| Group ID | Week 0 | Week 3 | Week 6 |
|---|---|---|---|
| A | 48 | 54 | 69 |
| B | 50 | 55 | 74 |
| C | 49 | 64 | 77 |
| D | 48 | 68 | 72 |
| E | 45 | 57 | 65 |
| F | 65 | 65 | 75 |

TABLE 32
% HDL/total cholesterol in transgenic mice

| Group ID | Week 0 | Week 3 | Week 6 |
|---|---|---|---|
| A | 24 | 25 | 31 |
| B | 25 | 24 | 31 |
| C | 24 | 30 | 35 |
| D | 25 | 40 | 37 |
| E | 23 | 34 | 39 |
| F | 41 | 37 | 42 |

TABLE 33
Triglyceride levels (mg/dL) in transgenic mice

| Group ID | Week 0 | Week 3 | Week 6 |
|---|---|---|---|
| A | 142 | 159 | 194 |
| B | 137 | 177 | 192 |
| C | 145 | 199 | 248 |
| D | 142 | 177 | 210 |
| E | 134 | 108 | 138 |
| F | 150 | 213 | 201 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.) (Nyblom, H. et al., Alcohol & Alcoholism 39: 336-339, 2004; Tietz N W (Ed): Clinical Guide to Laboratory Tests, 3rd ed. W. B. Saunders, Philadelphia, Pa., 1995). Plasma concentrations of ALT (alanine transaminase) and AST (aspartate transaminase) at weeks 0, 3 and 6 were measured and the results are presented in Tables 34 and 35 expressed in IU/L. Treatment with ISIS oligonucleotides or nicotinic acid did not adversely affect liver function, as transaminase levels were unchanged following extended oligonucleotide administration.

TABLE 34
Alanine transaminase levels (IU/L) of transgenic mice

| Group ID | Week 0 | Week 3 | Week 6 |
|---|---|---|---|
| A | 22 | 23 | 33 |
| B | 28 | 27 | 31 |
| C | 22 | 24 | 31 |
| D | 37 | 26 | 33 |
| E | 25 | 28 | 34 |
| F | 20 | 25 | 32 |

TABLE 35
Aspartate transaminase levels (IU/L) of transgenic mice

| Group ID | Week 0 | Week 3 | Week 6 |
|---|---|---|---|
| A | 40 | 36 | 84 |
| B | 38 | 41 | 62 |
| C | 36 | 35 | 56 |
| D | 52 | 45 | 91 |
| E | 61 | 43 | 64 |
| F | 37 | 41 | 60 |

### Body and Organ Weights

Body weights of the mice were measured weekly and are presented in Table 36. Liver, spleen and kidney weights were measured at the end of the study, and are presented in Table 37.

TABLE 36
Body weights of transgenic mice

| Group ID | Week 0 | Week 1 | Week 2 | Week 3 | Week 4 | Week 6 |
|---|---|---|---|---|---|---|
| A | 26 | 26 | 27 | 28 | 28 | 29 |
| B | 27 | 27 | 28 | 29 | 29 | 30 |
| C | 25 | 26 | 26 | 27 | 28 | 28 |
| D | 26 | 27 | 28 | 29 | 29 | 30 |
| E | 23 | 23 | 24 | 25 | 26 | 26 |
| F | 26 | 27 | 28 | 28 | 29 | 30 |

TABLE 37
Organ weights of transgenic mice

| Group ID | Liver | Kidney | Spleen |
|---|---|---|---|
| A | 1.35 | 0.39 | 0.09 |
| B | 1.48 | 0.39 | 0.10 |
| C | 1.46 | 0.35 | 0.09 |
| D | 1.58 | 0.38 | 0.11 |
| E | 1.22 | 0.37 | 0.09 |
| F | 1.51 | 0.39 | 0.09 |

### Example 8

### The Effect of Antisense Inhibition of CETP by ISIS 349517 in the huCETP Transgenic Mouse Model

The efficacy and tolerability of the compound ISIS 349517 was evaluated.

### Treatment

Male transgenic mice were maintained on a 12-hour light/dark cycle and were fed ad libitum the Western diet (TD88137; 42% cal from fat, 0.2% cholesterol; Harlan Laboratories, Indianapolis, Ind.). Animals were acclimated for at least 7 days in the research facility before initiation of the experiment and were maintained on this diet throughout the study period. Antisense oligonucleotides (ASOs) were prepared in phosphate buffered saline (PBS) and sterilized by filtering through a 0.2 micron filter. Oligonucleotides were dissolved in 0.9% PBS for injection.

The mice were divided into two groups of 4-6 mice each. One group received subcutaneous injections of ISIS 349517 at a dose of 25 mg/kg twice per week for 2 weeks. A saline-injected group served as the control group to which oligonucleotide-treated groups were compared.

Inhibition of CETP mRNA

Twenty four hours after the final dose, the animals were sacrificed and liver tissue was isolated.

Liver RNA was isolated for real-time PCR analysis of CETP. The primer probe set hCETP was used to measure CETP levels. As presented in Table 38, treatment with ISIS 349517 significantly reduced CETP mRNA expression. The RNA expression levels were normalized to murine Cyclophilin. The results are expressed as percent inhibition of CETP mRNA, relative to the PBS control.

TABLE 38
Percent inhibition of liver CETP mRNA expression in transgenic mice

| Treatment | % inhibition |
|---|---|
| ISIS 349517 | 84 |
| PBS | 0 |

### Inhibition of CETP Protein

Plasma CETP protein levels were measured by ELISA (Alpco). As presented in Table 39, treatment with ISIS 349517 significantly reduced CETP protein levels. The results are expressed as percent inhibition of CETP protein, relative to the PBS control.

TABLE 39
Percent inhibition of plasma CETP protein levels intransgenic mice

| Treatment | % inhibition |
|---|---|
| ISIS 349517 | 96 |
| PBS | 0 |

### Inhibition of CETP Activity

CETP activity levels were measured using a CETP activity assay kit (Roar Biomedical Inc., New York, N.Y.). As presented in Table 40, treatment with ISIS 349517 significantly reduced CETP activity. The results are expressed as percent inhibition of CETP activity, relative to the PBS control.

TABLE 40
Percent inhibition of CETP activity in transgenic mice

| | % |
|---|---|
| Treatment | inhibition |
| ISIS 349517 | 73 |
| PBS | 0 |

### Cholesterol and Triglyceride Levels

Plasma cholesterol were extracted at week 6 by the method of Bligh and Dyer (Bligh, E. G. and Dyer, W. J. Can. J. Biochem. Physiol. 37: 911-917, 1959) and measured with an Olympus clinical analyzer (Hitachi Olympus AU400e, Melville, N.Y.). HDL and LDL cholesterol were individually measured by HPLC. Triglyceride levels were measured with the use of a commercially available triglyceride kit (DCL Triglyceride Reagent; Diagnostic Chemicals Ltd., Charlottetown, Canada). The results are presented in Table 41 and are expressed in mg/dL. Treatment with ISIS 349517 resulted in 30% increase in HDL levels on week 3 compared to the PBS control.

TABLE 41
Cholesterol and triglyceride levels (mg/dL) in transgenic mice

| | Total | LDL | HDL | HDL % at | Triglyc- |
|---|---|---|---|---|---|
| Treatment | cholesterol | Cholesterol | cholesterol | week 0 | erides |
| PBS | 143 | 36 | 109 | 11 | 59 |
| ISIS 349517 | 212 | 48 | 155 | 39 | 58 |

### Liver Function

To evaluate the effect of ISIS 349517 on hepatic function, plasma concentrations of transaminases were measured using an automated clinical chemistry analyzer (Hitachi Olympus AU400e, Melville, N.Y.) (Nyblom, H. et al., Alcohol & Alcoholism 39: 336-339, 2004; Tietz N W (Ed): Clinical Guide to Laboratory Tests, 3rd ed. W. B. Saunders, Philadelphia, Pa., 1995). Plasma concentrations of ALT (alanine transaminase) and AST (aspartate transaminase) were measured and the results are presented in Table 42 expressed in IU/L. Treatment with ISIS oligonucleotides did not adversely affect liver function, as transaminase levels were unchanged following extended CETP oligonucleotide administration.

TABLE 42
Transaminase levels (IU/L) of transgenic mice

| Treatment | ALT | AST |
|---|---|---|
| PBS | 26 | 41 |
| ISIS 349517 | 26 | 38 |

### Example 9

### Dose Response Effect of Antisense Inhibition of CETP by ISIS 349517 in the huCETP Transgenic Mouse Model

The efficacy and tolerability of an ISIS oligonucleotide at different doses was evaluated.

### Treatment

Male transgenic mice were maintained on a 12-hour light/dark cycle and were fed ad libitum the Western diet (TD88137; 42% cal from fat, 0.2% cholesterol; Harlan Laboratories, Indianapolis, Ind.). Animals were acclimated for at least 7 days in the research facility before initiation of the experiment and were maintained on this diet throughout the study period. Antisense oligonucleotides (ASOs) were prepared in buffered saline (PBS) and sterilized by filtering through a 0.2 micron filter. Oligonucleotides were dissolved in 0.9% PBS for injection.

Three groups received subcutaneous injections of ISIS 349517 at a dose of 1.5 mg/kg/week, 5 mg/kg/week or 15 mg/kg/week for 6 weeks. Another group received subcutaneous injections of control oligonucleotide ISIS 141923 at a dose of 50 mg/kg/week for 6 weeks. One group of mice received subcutaneous injections of PBS for 6 weeks. The PBS-injected group served as the control group to which treatment groups were compared.

Inhibition of CETP mRNA

Twenty four hours after the final dose, the animals were sacrificed and liver tissue was isolated. Liver RNA was isolated for real-time PCR analysis of CETP. The primer probe sets hCETP and hCETP2_LTS00182 were individually used to measure CETP levels. As presented in Table 43, treatment with ISIS 349517 reduced CETP mRNA expression in a dose-dependent manner. Both primer probe sets produced similar results. The RNA expression levels were normalized to murine Cyclophilin. The results are expressed as percent inhibition of CETP mRNA, relative to the PBS control.

TABLE 43
Percent inhibition of liver CETP mRNA
expression in transgenic mice

| | Dose | PPset | PPset |
|---|---|---|---|
| ISIS No. | (mg/kg/wk) | hCETP | hCETP2_LTS00182 |
| 349517 | 1.5 | 59 | 59 |
| | 5 | 87 | 87 |
| | 15 | 91 | 95 |
| | 50 | 92 | 96 |
| 141923 | 50 | 0 | 0 |

Inhibition of mRNA Expression of Genes Implicated in Cardiovascular Disorders

Liver RNA was isolated for real-time PCR analysis of ABCA1, ApoAI, and SR-B1. ATP-binding cassette transporter ABCA1 (member 1 of human transporter sub-family ABCA), also known as the cholesterol efflux regulatory protein (CERP) is a protein which in humans is encoded by the ABCA1 gene (Luciani, M. F. et al., Genomics. 1994. 21: 150-9). This transporter is a major regulator of cellular cholesterol and phospholipid homeostasis. Apolipoprotein A-I is a protein that in humans is encoded by the APOA1 gene (Breslow, J. L. et al., Proc. Natl. Acad. Sci. USA 1982. 79: 6861-5). Apolipoprotein A-I is the major protein component of HDL in plasma. The protein promotes cholesterol efflux from tissues to the liver for excretion. Scavenger receptor class B, type I (SR-BI) is an integral membrane protein found in numerous cell types/tissues, including the liver and adrenal (Acton, S. L. et al., J. Biol. Chem. 1994. 269: 21003-21009). It is best known for its role in facilitating the uptake of cholesteryl esters from high-density lipoproteins in the liver. Therefore, all three genes have a role in HDL regulation.

The primer probe set RTS1204 (forward sequence GGACTTGGTAGGACGGAACCT, designated herein as SEQ ID NO: 122, reverse sequence ATCCTCATCCTCGTCATTCAAAG, designated herein as SEQ ID NO: 123, probe sequence AGGCCCAGACCTGTAAAGGCGAAGX, with X a fluorophore, designated herein as SEQ ID NO: 124) was used to measure abca1 levels. The primer probe set RTS14737 (forward sequence ACTCTGGGTTCAACCGTTAGTCA, designated herein as SEQ ID NO: 125, reverse sequence TATCCCAGAAGTCCCGAGTCAA, designated herein as SEQ ID NO: 126, probe sequence CTGCAGGAACGGCTGGGCCCX, with X a fluorophore, designated herein as SEQ ID NO: 127) was used to measure apoA1 levels. The primer probe set mSRB-1 (forward sequence TGACAACGACACCGTGTCCT, designated herein as SEQ ID NO: 128, reverse sequence ATGCGACTTGTCAGGCTGG, designated herein as SEQ ID NO: 129, probe sequence CGTGGAGAACCGCAGCCTCCATTX, with X a fluorophore, designated herein as SEQ ID NO: 130) was used to measure sr-b1 levels. The results are presented in Table 44 as percentage increase or decrease in the respective expression levels compared to the PBS control. Both primer probe sets produced similar results. The RNA expression levels were normalized to murine Cyclophilin. Treatment with ISIS 349517 caused an increase in ApoA1 and SR-B1 levels.

TABLE 44
Percent inhibition of liver mRNA expression in
transgenic mice by ISIS oligonucleotides

| | Dose | | | |
|---|---|---|---|---|
| ISIS No. | (mg/kg/wk) | ABCA1 | ApoA1 | SR-B1 |
| 349517 | 1.5 | +6 | −34 | +33 |
| | 5 | +15 | −13 | +34 |
| | 15 | −6 | +52 | +50 |
| | 50 | −15 | +136 | +42 |
| 141923 | 50 | +16 | −50 | +37 |

### Inhibition of CETP Protein

Plasma CETP protein levels were measured by ELISA (Alpco). The results are expressed as percent inhibition of CETP protein, relative to the PBS control. As presented in Table 45, treatment with ISIS 349517 significantly reduced CETP protein levels.

TABLE 45
Percent inhibition of plasma CETP protein levels in
transgenic mice by ISIS nucleotides

| ISIS | Dose | % |
|---|---|---|
| No. | (mg/kg/wk) | inhibition |
| 349517 | 1.5 | 58 |
| | 5 | 74 |
| | 15 | 81 |
| | 50 | 80 |
| 141923 | 50 | 7 |

### Inhibition of CETP Activity

CETP activity levels were measured using a CETP activity assay kit (Roar Biomedical Inc.). As presented in Table 46, treatment with ISIS 349517 significantly reduced CETP activity by 77% at the maximum dose of 50 mg/kg/week.

TABLE 46
Percent inhibition of CETP activity in transgenic
mice by ISIS oligonucleotides

| ISIS | Dose | % |
|---|---|---|
| No. | (mg/kg/wk) | inhibition |
| 349517 | 1.5 | 33 |
| | 5 | 59 |
| | 15 | 76 |
| | 50 | 77 |
| 141923 | 50 | 2 |

### Cholesterol and Triglyceride Levels

Plasma cholesterol were extracted at week 6 by the method of Bligh and Dyer (Bligh, E. G. and Dyer, W. J. Can. J. Biochem. Physiol. 37: 911-917, 1959) and measured with an Olympus clinical analyzer (Hitachi Olympus AU400e, Melville, N.Y.). HDL and LDL cholesterol were individually measured by HPLC. Triglyceride levels were measured with the use of a commercially available triglyceride kit (DCL Triglyceride Reagent; Diagnostic Chemicals Ltd.). The results are presented in Tables 47-51 and are expressed in mg/dL.

TABLE 47
Total cholesterol levels (mg/dL) in transgenic
mice treated with ISIS oligonucleotides

| | Dose | | | |
|---|---|---|---|---|
| | (mg/kg/wk) | Week 0 | Week 3 | Week 6 |
| PBS | — | 161 | 162 | 183 |
| ISIS 349517 | 1.5 | 154 | 171 | 180 |
| | 5 | 164 | 172 | 202 |
| | 15 | 152 | 185 | 217 |
| | 50 | 145 | 184 | 170 |
| ISIS 141923 | 50 | 153 | 164 | 180 |

TABLE 48
HDL cholesterol levels (mg/dL) in transgenic
mice treated with ISIS oligonucleotides

| | Dose | | | |
|---|---|---|---|---|
| | (mg/kg/wk) | Week 0 | Week 3 | Week 6 |
| PBS | — | 85 | 114 | 127 |
| ISIS 349517 | 1.5 | 80 | 117 | 132 |
| | 5 | 88 | 130 | 151 |
| | 15 | 86 | 141 | 164 |
| | 50 | 77 | 139 | 123 |
| ISIS 141923 | 50 | 87 | 115 | 124 |

TABLE 49
% change in HDL levels compared to total cholesterol in
transgenic mice treated with ISIS oligonucleotides

| | Dose | | |
|---|---|---|---|
| | (mg/kg/wk) | Week 3 | Week 6 |
| PBS | — | −5 | 0 |
| ISIS 349517 | 1.5 | 12 | 24 |
| | 5 | 11 | 23 |
| | 15 | 26 | 42 |
| | 50 | 43 | 37 |
| ISIS 141923 | 50 | −17 | −23 |

TABLE 50
LDL cholesterol levels (mg/dL) in transgenic
mice treated with ISIS oligonucleotides

| | Dose | | | |
|---|---|---|---|---|
| | (mg/kg/wk) | Week 0 | Week 3 | Week 6 |
| PBS | — | 63 | 41 | 47 |
| ISIS 349517 | 1.5 | 58 | 35 | 38 |
| | 5 | 62 | 31 | 36 |
| | 15 | 54 | 32 | 40 |
| | 50 | 59 | 33 | 38 |
| ISIS 141923 | 50 | 54 | 42 | 47 |

TABLE 51
Triglyceride levels (mg/dL) in transgenic
mice treated with ISIS oligonucleotides

| | Dose | | | |
|---|---|---|---|---|
| | (mg/kg/wk) | Week 0 | Week 3 | Week 6 |
| PBS | — | 58 | 45 | 96 |
| ISIS 349517 | 1.5 | 63 | 50 | 91 |
| | 5 | 57 | 51 | 85 |
| | 15 | 47 | 48 | 85 |
| | 50 | 41 | 49 | 60 |
| ISIS 141923 | 50 | 49 | 58 | 88 |

### Liver Function

To evaluate the effect of ISIS 349517 on hepatic function, plasma concentrations of transaminases were measured using an automated clinical chemistry analyzer (Hitachi Olympus AU400e, Melville, N.Y.) (Nyblom, H. et al., Alcohol & Alcoholism 39: 336-339, 2004; Tietz N W (Ed): Clinical Guide to Laboratory Tests, 3rd ed. W. B. Saunders, Philadelphia, Pa., 1995). Plasma concentrations of ALT (alanine transaminase) and AST (aspartate transaminase) were measured and the results are presented in Tables 52-53 expressed in IU/L. Treatment with ISIS oligonucleotides did not adversely affect liver function, as transaminase levels were unchanged following extended CETP oligonucleotide administration.

TABLE 52
ALT levels (IU/L) of transgenic mice
treated with ISIS oligonucleotides

| | Dose | | | |
|---|---|---|---|---|
| | (mg/kg/wk) | Week 0 | Week 3 | Week 6 |
| PBS | — | 28 | 27 | 46 |
| ISIS | 1.5 | 20 | 18 | 54 |
| 349517 | 5 | 26 | 25 | 49 |
| | 15 | 24 | 27 | 36 |
| | 50 | 27 | 33 | 76 |
| ISIS | 50 | 27 | 27 | 45 |
| 141923 | | | | |

TABLE 53
AST levels (IU/L) of transgenic
mice treated with ISIS oligonucleotides

| | Dose | | | |
|---|---|---|---|---|
| | (mg/kg/wk) | Week 0 | Week 3 | Week 6 |
| PBS | — | 28 | 52 | 92 |
| ISIS | 1.5 | 38 | 43 | 133 |
| 349517 | 5 | 40 | 46 | 246 |
| | 15 | 24 | 47 | 70 |
| | 50 | 27 | 52 | 171 |
| ISIS | 50 | 26 | 46 | 107 |
| 141923 | | | | |

### Glucose Levels

Plasma glucose values were determined using a Beckman Glucose Analyzer II (Beckman Coulter) by a glucose oxidase method (Lott, J. A. et al., Clin. Chem. 21: 1754-1760, 1975). The results are presented in Table 54, expressed in mg/dL.

TABLE 54
Glucose levels in transgenic mice
treated with ISIS oligonucleotides

| | Dose | | | |
|---|---|---|---|---|
| | (mg/kg/wk) | Week 0 | Week 3 | Week 6 |
| PBS | — | 366 | 263 | 364 |
| ISIS | 1.5 | 375 | 288 | 348 |
| 349517 | 5 | 354 | 287 | 346 |
| | 15 | 339 | 271 | 329 |
| | 50 | 339 | 269 | 255 |
| ISIS | 50 | 311 | 256 | 305 |
| 141923 | | | | |

### Example 10

### Effect of Antisense Inhibition of CETP in the huCETP Transgenic LDL Receptor Knockout Mouse Model

In human CETP transgenic mice, the bulk of the cholesterol in plasma is associated with HDL and, in this model, cholesteryl ester (CE) transferred from HDL to VLDL/LDL by CETP is rapidly cleared by the LDL receptor (LDLr) (Zhou, H. et al., Biochim Biophys. Acta. 1761: 14821488, 2006). The human CETP transgenic, LDLr knockout mouse model used in this example is an animal model for cardiovascular disease. In this model, clearance of LDL is delayed due to the absence of LDLr leading to the inability of the mice to clear CE via the LDL pathway (Plump, A. S. et al., Arterioscler. Thromb. Vasc. Biol. 19: 1105-1110, 1999). This human CETP transgenic, LDLr knockout mouse is helpful in assessing the effect of antisense inhibition of CETP on HDL composition as the model has a proatherogenic lipoprotein profile where most of the cholesterol is found in VLDL and LDL.

### Treatment

Male transgenic mice were maintained on a 12-hour light/dark cycle and were fed ad libitum the Western diet (TD88137; 42% cal from fat, 0.2% cholesterol; Harlan Laboratories, Indianapolis, Ind.). Animals were acclimated for at least 7 days in the research facility before initiation of the experiment and were maintained on this diet throughout the study period. Antisense oligonucleotides (ASOs) were prepared in buffered saline (PBS) and sterilized by filtering through a 0.2 micron filter. Oligonucleotides were dissolved in 0.9% PBS for injection.

One group of mice received subcutaneous injections of ISIS 349517 at a dose of 15 mg/kg/week, for 6 weeks. Another group received subcutaneous injections of control oligonucleotide ISIS 141923 at a dose of 15 mg/kg/week for 6 weeks. One group of mice received subcutaneous injections of PBS for 6 weeks. The PBS-injected group served as the control group to which treatment groups were compared.

At the end of the treatment period, plasma samples were taken for further analysis.

### Effect on HDL Composition

The VLDL+IDL, LDL and HDL lipoprotein sub-fractions were separated by ultracentrifugation, based on their relative densities. Pooled plasma samples from all the groups of 5004 each was added to a 2 mL Beckman Quick-Seal centrifuge tube and the density adjusted to 1.019 g/mL with potassium bromide (KBr). The samples were then spun in a Beckman TL-100 ultracentrifuge for 4 hours at 100,000 rpm at 4 degrees Celsius. The top of the tube containing the VLDL+IDL sub-fraction was sliced off and collected.

The bottom fraction was collected in a new tube; the density was re-adjusted to 1.063 g/ml with KBr and re-spun at 100,000 rpm at 4 degrees Celsius for 5 hours to concentrate the LDL sub-fraction. The top of the tube containing the LDL sub-fraction was collected. Once again, the bottom fraction was placed in a new tube and the density was now adjusted to 1.21 g/ml with KBr to concentrate the HDL sub-fraction. The tubes were spun at 100,000 rpm at 4 degrees Celsius for 6 hours. The HDL sub-fraction was finally collected. All of the lipoprotein sub-fractions were dialyzed overnight in PBS to remove excess KBr.

The sub-fractions were then assayed for total cholesterol and triglyceride (TG) using enzymatic assays from Roche (Basel, Switzerland). Phospholipid (PL) and free cholesterol (FC) were measured using enzymatic assays from Wako Chemicals USA (Richmond, Va.). Protein (Pro) concentration was measured by Fisher Scientific's BCA assay (Pittsburgh, Pa.). Cholesteryl ester (CE) was calculated as (total cholesterol−free cholesterol)×1.67 (CE is cholesterol plus a fatty acid, the 1.67 adjustment takes into account the increase in mass the fatty acid will add (Rudel L L et al, JCI 100 (1); 74-83)). The S/C ratio is the ratio of the surface components (Pro, FC, and PL) to the core components (CE and TG).

The concentration (%) of each component in the HDL fraction is presented in Table 55. The data demonstrate that antisense oligonucleotide inhibition of CETP in this mouse model decreases CETP-mediated lipid transfer to and from the HDL particle. The decrease in TG transferred to HDL resulted in a decrease in the TG component of HDL from 46% to 13% making the HDL particle smaller and denser. The decrease in CE transferred from HDL resulted in an increase in the CE component of HDL from 17.4% to 38%. Additionally, the S/C ratio is increased, indicating a shift to a smaller HDL particle, after antisense oligonucleotide inhibition of CETP. Accordingly, in this model, inhibiting the CETP-mediated transfer of TG to HDL, and CE from HDL, leads to the formation of smaller, denser, and CE rich HDL.

The overall composition of the smaller, denser, CE-rich HDL formed in the mice after CETP inhibition is more similar to the active HDL particle found in a normal, healthy human (Childs, Kinzler, and Vogelstein, The Metabolic and Molecular Bases of Inherited Disease 8^{th} edition, Vol 2, pg 2707) than the large, TG-rich HDL formed in the control mice. The larger, TG-rich HDL found in the control groups are more analogous to that found in patients suffering from hypertriglyceridemia and metabolic syndrome (Deckelbaum R J ATVB 4:3 225-231). HDL from patients suffering from these diseases display a diminished capacity for reverse cholesterol transport and a reduction in anti-inflammatory and antioxidant capability (Brites et al. Archives of Medical Research 35 (2004) 235-240, Kontush, Nature Clinical Practice 3:3 144-153).

Accordingly, the formation of smaller, denser HDL, CE-rich HDL particles are thought to indicate an active HDL particle. Increased HDL activity facilitates cholesterol delivery and cholesterol removal (Skeggs, J. W. and Morton, R. E. J. Lipid Res. 43: 1264-1274, 2002).

TABLE 55
Effect of antisense inhibition of CETP on HDL composition
in the CETP transgenic LDLr−/− mouse model

| | | | | | | S/C |
|---|---|---|---|---|---|---|
| Group/Class | Pro % | PL % | FC % | CE % | TG % | Ratio |
| PBS | 29.7 | 4.7 | 2.5 | 17.4 | 45.7 | 0.59 |
| ISIS 141923 | 24.8 | 8.4 | 3.1 | 16.6 | 47.1 | 0.57 |
| ISIS 349517 | 31.3 | 15.6 | 2.1 | 38.1 | 12.8 | 0.96 |

1.-2. (canceled)

3. A method of increasing HDL levels or HDL activity in an animal comprising administering to the animal a compound comprising a modified oligonucleotide 10 to 30 linked nucleosides in length targeted to CETP, wherein HDL levels or HDL activity is increased in the animal.

4. (canceled)

5. A method of reducing a LDL level, TG level, glucose level and/or LDL/HDL ratio in an animal comprising administering to the animal a compound comprising a modified oligonucleotide 10 to 30 linked nucleosides in length targeted to CETP, wherein the LDL level, TG level, glucose level and/or LDL/HDL ratio is reduced in the animal.

6. The method of claim 5, wherein the reduction in LDL level, TG level, glucose level and/or LDL/HDL ratio ameliorates a metabolic or cardiovascular disease in an animal.

7. The method of claim 6, wherein the metabolic or cardiovascular disease is aneurysm, angina, arrhythmia, atherosclerosis, cerebrovascular disease, coronary heart disease, hypertension, dyslipidemia, hyperlipidemia hypercholesterolemia, obesity, diabetes, non-alcoholic fatty liver disease (NAFLD), hyperfattyacidemia, metabolic syndrome, or a combination thereof.

8. The method of claim 5, wherein the compound consists of a single-stranded modified oligonucleotide.

9. The method of claim 5, wherein the animal is a human.

10. The method of claim 5, wherein the compound is a first agent and further comprising administering a second agent.

11.-14. (canceled)

15. The method of claim 5, wherein the modified oligonucleotide has a nucleobase sequence at least 90%, at least 95% or 100% complementary to any of SEQ ID NO: 1-4 as measured over the entirety of said modified oligonucleotide.

16.-17. (canceled)

18. The method of claim 5, wherein at least one internucleoside linkage of said modified oligonucleotide is a modified internucleoside linkage, at least one nucleoside of said modified oligonucleotide comprises a modified sugar and/or at least one nucleoside of said modified oligonucleotide comprises a modified nucleobase.

19. The method of claim 18, wherein each internucleoside linkage is a phosphorothioate internucleoside linkage, at least one modified sugar is a bicyclic sugar and the modified nucleobase is a 5-methylcytosine.

20.-23. (canceled)

24. The method of claim 18, wherein at least one modified sugar comprises a 2′-O-methoxyethyl or a 4′-(CH₂)ₙ—O-2′ bridge, wherein n is 1 or 2.

25.-26. (canceled)

27. The method of claim 5, wherein the modified oligonucleotide consists of 20 linked nucleosides.

28. The method of claim 5, wherein the modified oligonucleotide comprises:
a gap segment consisting of linked deoxynucleosides;
a 5′ wing segment consisting of linked nucleosides;
a 3′ wing segment consisting of linked nucleosides;
wherein the gap segment is positioned between the 5′ wing segment and the 3′ wing segment and wherein each nucleoside of each wing segment comprises a modified sugar.

29. The method of claim 5, wherein the modified oligonucleotide consists of 20 linked nucleosides, has a nucleobase sequence comprising at least 8 contiguous nucleobases complementary to an equal length portion of any of SEQ ID NO: 1-4 and comprises:
a gap segment consisting of ten linked deoxynucleosides;
a 5′ wing segment consisting of five linked nucleosides;
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

30.-32. (canceled)

33. The method of claim 3, wherein the HDL level and/or HDL activity is increased by at least 5%, 10%, 20%, 30%, 35%, 40%, 45%, 50%, 55%, 60%, 65%, 70%, 75%, 80%, 85%, 90%, 95% or 100%.

34.-37. (canceled)

38. A compound comprising a modified oligonucleotide consisting of 10 to 30 linked nucleosides and having a nucleobase sequence comprising at least 8 contiguous nucleobases of any of SEQ ID NO: 41-114.

39. The compound of claim 38, wherein the nucleobase sequence of the modified oligonucleotide is at least 95% or 100% complementary to any of SEQ ID NO: 1-4.

40.-41. (canceled)

42. The compound of claim 38, wherein at least one internucleoside linkage is a modified internucleoside linkage, at least one nucleoside comprises a modified sugar and/or at least one nucleoside comprises a modified nucleobase.

43. The compound of claim 42, wherein each internucleoside linkage is a phosphorothioate internucleoside linkage, the modified sugar is a bicyclic sugar and the modified nucleobase is a 5-methylcytosine.

44.-47. (canceled)

48. The compound of claim 42, wherein at least one modified sugar comprises a 2′-O-methoxyethyl or a 4′-(CH₂)ₙ—O-2′ bridge, wherein n is 1 or 2.

49.-56. (canceled)
